(19) United States
(12) Patent Application Publication
(10) Pub. No.: US 2023/0255979 A1
(43) Pub. Date: Aug. 17, 2023

(54) METHODS OF TREATING ACUTE RESPIRATORY DISORDERS

(71) Applicant: Bristol-Myers Squibb Company, Princeton, NJ (US)

(72) Inventors: Alan F. Corin, Sudbury, MA (US); John Michael Ellis, Needham, MA (US); Francisco Ramirez-Valle, New York, NY (US); Kofi Agyare Mensah, Medford, MA (US)

(21) Appl. No.: 18/017,121

(22) PCT Filed: Jul. 22, 2021
(86) PCT No.: PCT/US21/42726

§ 371 Date: Jan. 20, 2023

Related U.S. Application Data
(60) Provisional application No. 63/056,348, filed on Jul. 24, 2020.

(51) Int. Cl. A61K 31/551; A61P 31/14; A61K 9/00

(52) U.S. Cl. CPC A61K 31/551; A61P 31/14; A61K 9/0053

(57) ABSTRACT

The present disclosure provides methods of treating, stabilizing, or lessening the severity or progression of an acute respiratory disorder.

### CROSS REFERENCE TO RELATED APPLICATIONS

The present application claims priority to U.S. Provisional Application No. 63/056,348, filed Jul. 24, 2020, entitled “METHODS OF TREATING ACUTE RESPIRATORY DISORDERS”, the entirety of which is hereby incorporated by reference.

### FIELD OF THE INVENTION

The present invention provides methods of treating, stabilizing, or lessening the severity or progression of an acute respiratory disorder.

### BACKGROUND

In December 2019, Wuhan, the capital of Hubei province in China, became the center of an outbreak of pneumonia of unknown cause. By January 2020, scientists had isolated a novel coronavirus, severe acute respiratory syndrome coronavirus 2 (SARS-CoV-2; previously known as 2019-nCoV), from patients with virus-infected pneumonia, (see Phelan, et al. JAMA Cardiol. 2020, E1-E2; Gorbalenya, et al. Nature Microbiology 2020, 5, 536-544), which was later designated coronavirus disease 2019 (COVID-19) in February 2020 by the World Health Organization (WHO 2020). While the Food and Drug Administration (FDA) has granted Emergency Use Authorization (EUA) for several agents, there are presently no FDA-approved therapies or vaccines for the treatment or prevention of diseases associated with SARS-CoV-2.

### SUMMARY OF THE INVENTION

The present disclosure provides methods of treating, stabilizing, or lessening the severity or progression of a disease or disorder associated with a coronavirus. In some embodiments, the present disclosure provides methods of treating, stabilizing, or lessening the severity or progression of a disease or disorder associated with SARS-CoV-2. In some embodiments, the present disclosure provides a method of treating a respiratory disease or disorder associated with SARS-CoV-2. In some such embodiments, the disease or disorder associated with SARS-CoV-2 is COVID-19.

In some embodiments, the present disclosure provides a method of treating, stabilizing, or lessening the severity or progression of a disease or disorder associated with a coronavirus (e.g., SARS-CoV-2), the method comprising inhibiting the activity of mitogen-activated protein kinase-activated protein kinase 2 (also known as “MAP-kinase-activated protein kinase 2”, “MAPKAPK2”, or “MK2”), or a mutant thereof.

In some embodiments, the present disclosure provides a method of treating, stabilizing, or lessening the severity or progression of a disease or disorder associated with a coronavirus (e.g., SARS-CoV-2), the method comprising administering to a patient in need thereof an effective amount of a compound of formula I, or a pharmaceutically acceptable salt thereof. In some embodiments, the effective amount of a compound of formula I, or a pharmaceutically acceptable salt thereof, is an amount effective to inhibit the activity of MK2, or a mutant thereof.

Accordingly, in some embodiments, the present disclosure provides a method of treating, stabilizing, or lessening the severity or progression of a disease or disorder associated with a coronavirus (e.g., SARS-CoV-2), the method comprising administering to a patient in need thereof a compound of formula I, or a pharmaceutically acceptable salt thereof, in an amount effective to inhibit the activity of MK2, or a mutant thereof.

In some embodiments, the present disclosure provides a use of an inhibitor of activity of MK2, or a mutant thereof, for treating, stabilizing, or lessening the severity or progression of a disease or disorder associated with a coronavirus (e.g., SARS-CoV-2).

In some embodiments, the present disclosure provides a method of treating, stabilizing, or lessening the severity or progression of a disease or disorder associated with a coronavirus (e.g., SARS-CoV-2), the method comprising administering to a patient in need thereof an effective amount of a compound of formula I:

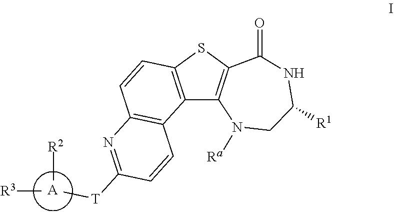

or a pharmaceutically acceptable salt thereof, wherein:
Ring A is phenyl, a 5-6 membered monocyclic heteroaryl ring having 1-3 nitrogen atoms, or an 8-14 membered bicyclic heteroaryl ring having 1-4 heteroatoms independently selected from nitrogen, oxygen, and sulfur;
T is a bivalent moiety selected from —N(R)—, —O—, —S—, —S(O)—, —SO₂—, —C(S)—, —Si(R⁴)₂—, —P(R⁵)—, —P(O)₂—, or a bivalent saturated straight or branched 1-3 membered hydrocarbon chain, wherein the hydrocarbon chain is optionally substituted with oxo or —OR;
each R is independently hydrogen or an optionally substituted C₁₋₆ aliphatic, or:
two R groups on the same nitrogen are taken together with the nitrogen to form a 3-7 membered saturated or partially unsaturated heterocyclic ring having 1-3 heteroatoms selected from nitrogen, oxygen, or sulfur;
R^{a} is hydrogen or an optionally substituted C₁₋₆ aliphatic;
R¹ is —R or —(CH₂)ₚR^{x};
p is 0, 1, 2, or 3;
R^{x} is —CN, —NO₂, halogen, —OR, —SR, —N(R)₂, —C(O)N(R)₂, —C(O)OR, —C(O)R, —N(R)C(O)R, —SO₂N(R)₂, or —N(R)SO₂;
R² is halogen, —CN, —SR^{y}, —S(O)R^{y}, —SO₂R^{y}, —OSO₂R^{y}, —OC(O)R^{y}, or —OP(O)₂OR^{y};
each R^{y} is independently selected from optionally substituted C₁₋₆ aliphatic or optionally substituted phenyl;
R³ is hydrogen, optionally substituted C₁₋₆ aliphatic, —CN, —NO₂, halogen, —OR, —N(R)₂, —C(O)N(R)₂, —C(O)OR, -Cy, —C(O)N(R)-Cy, —C(O)-Cy, —O-Cy, —O—(CH₂)ₙ-Cy, —(CH₂)ₙ—O-Cy, —(CH₂)ₘN(R)₂, —(CH₂)ₘOR, —N(R)-Cy, —N(R)—(CH₂)ₙ-Cy, —(CH₂)ₙ—N(R)-Cy, or —(CH₂)ₘ-Cy;
each R⁴ is independently hydrogen, —OR, C₁₋₆ aliphatic, phenyl, or a 5-6 membered heteroaryl ring having 1-3 heteroatoms independently selected from nitrogen, oxygen, and sulfur;
each R⁵ is independently —OR, C₁₋₆ aliphatic, phenyl, or a 5-6 membered heteroaryl ring having 1-3 heteroatoms independently selected from nitrogen, oxygen, and sulfur;
each of m and n is independently 0-4; and
each Cy is independently an optionally substituted ring selected from a 3-9 membered saturated or partially unsaturated monocyclic carbocyclic ring, a 3-9 membered saturated or partially unsaturated monocyclic heterocyclic ring having 1-3 heteroatoms independently selected from nitrogen, oxygen, and sulfur, phenyl, a 5-6 membered heteroaryl ring having 1-3 heteroatoms independently selected from nitrogen, oxygen, and sulfur, a 7-12 membered saturated or partially unsaturated fused or bridged bicyclic carbocyclic ring, or a 6-12 membered saturated or partially unsaturated fused or bridged bicyclic heterocyclic ring having 1-3 heteroatoms independently selected from nitrogen, oxygen, and sulfur.

### BRIEF DESCRIPTION OF THE DRAWINGS

FIG. 1A is a bar graph depicting the dose dependent inhibition of TNF-α in peripheral blood mononuclear cells (PMBCs) following lipopolysaccharide (LPS) stimulation with and without compound I-82 for 24 hours. FIG. 1B is a bar graph depicting the dose dependent inhibition of TNF-α in PMBCs following staphylococcal enterotoxin-B (SEB)/interleukin-2 (IL-2) treatment with and without compound I-82 (μM) for 3 days.

FIG. 2 is a bar graph depicting the dose dependent inhibition of TNF-α in monocytes following LPS stimulation for 23 hours (compound I-82 is in μM).

FIG. 3 is a bar graph depicting the dose-dependent inhibition of TNF-α in monocytes and macrocytes after treatment with compound I-82 (μM) for 4 h or 24 h following LPS stimulation for 3 or 23 hours.

FIG. 4A is a bar graph depicting the dose-dependent inhibition of IL-6 in monocytes and macrocytes after treatment with compound I-82 (μM) for 4 h or 24 h and following LPS stimulation for 3 or 23 hours. FIG. 4B is a bar graph depicting the dose-dependent inhibition of IL-1β in monocytes and macrocytes after treatment with compound I-82 (μM) for 4 h or 24 h and following LPS stimulation for 3 or 23 hours.

FIG. 5 depicts the inhibition of gene expression by compound I-82 of MCP-1 (FIG. 5A), IL-6 (FIG. 5B), and Il-1β (FIG. 5C).

FIG. 6 depicts the inhibition of gene expression by compound I-82 of TNF-α (FIG. 6A) and GM-CSF (FIG. 6B). FIG. 6C is a bar graph demonstrating that compound I-82 does not inhibit gene expression of ZFP36 (TTP).

FIG. 7 is a line graph depicting dose-dependent target engagement of compound I-82 in the multiple ascending dose study in healthy volunteers. Solid lines represent mean target engagement in subjects administered active treatment. Dotted line ( [symbol] ) represents mean target engagement in subjects administered placebo.

FIG. 8A is a line graph depicting the inhibition of TNF-α following ex vivo LPS stimulation in the multiple ascending dose study in healthy volunteers. Solid lines represent mean TNF-α inhibition in subjects administered active treatment. Dotted line ( [symbol] ) represents mean TNF-α inhibition in subjects administered placebo.

FIG. 8B is a line graph depicting the inhibition of IL-6 following ex vivo LPS stimulation in subjects administered placebo (dotted lines) or 150 mg of compound I-82 (solid lines) in the multiple ascending dose study in healthy volunteers. Individual level data is represented.

FIG. 8C is a line graph depicting the inhibition of MIP-1A following ex vivo LPS stimulation in subjects administered placebo (dotted lines) or 150 mg of compound I-82 (solid lines) in the multiple ascending dose study in healthy volunteers. Individual level data is represented.

FIG. 8D is a line graph depicting the inhibition of MIP-1B following ex vivo LPS stimulation in subjects administered placebo (dotted lines) or 150 mg of compound I-82 (solid lines) in the multiple ascending dose study in healthy volunteers. Individual level data is represented.

### DEFINITIONS

Compounds provided herein include those described generally above, and are further illustrated by the classes, subclasses, and species disclosed herein. As used herein, the following definitions shall apply unless otherwise indicated. For purposes of this disclosure, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “carbocyclic”, “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “carbocyclic” (or “cycloaliphatic” or “carbocycle” or “cycloalkyl”) refers to a monocyclic C₃-C₈ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bridged bicyclics include:

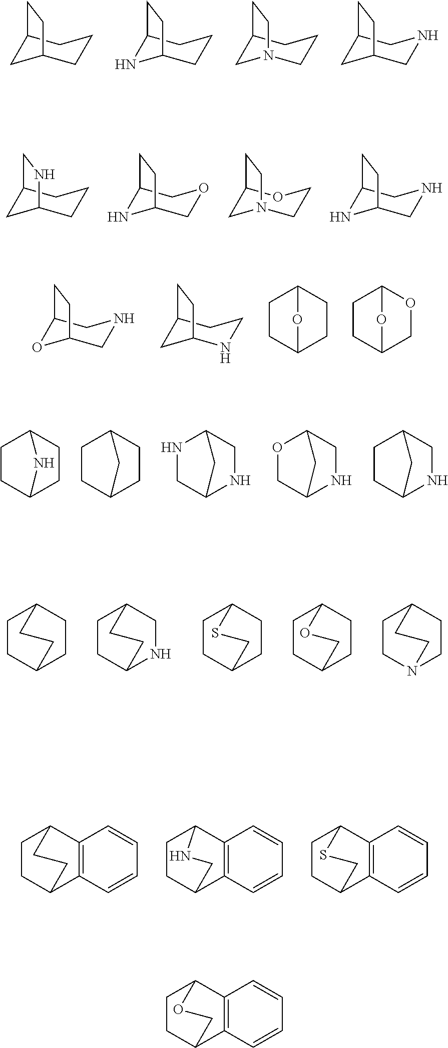

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

As used herein, the term “cyclopropylenyl” refers to a bivalent cyclopropyl group of the following structure:

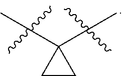 [SMILES: *C1(*)CC1]

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present disclosure, “aryl” refers to an aromatic ring system and exemplary groups include phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Exemplary heteroaryl groups include thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Exemplary groups include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the disclosure may contain “optionally substituted” moieties. In general, the term “substituted,” whether preceded by the term “optionally” or not, means that one or more hydrogens of the designated moiety are replaced with a suitable substituent. “Substituted” applies to one or more hydrogens that are either explicit or implicit from the structure (e.g.,

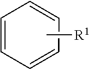

refers to at least

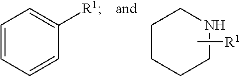

refers to at least

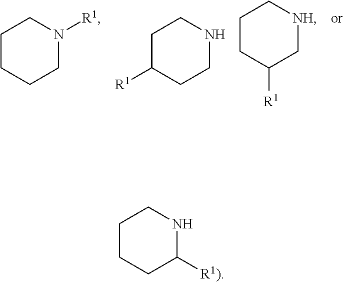

Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂, —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O (“oxo”), ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R^{●} include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁-4 aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “about” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed disclosure. As an example, when the term “about” is used in combination with a certain number of days, it includes said specific number of days plus or minus 1 day, e.g., “about 6 days” includes any number of days between 5 and 7. It is to be understood that the value to which the modifier “about” refers is itself also specifically, and preferably, disclosed.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate. As used herein, a “therapeutically effective amount” means an amount of a substance (e.g., a therapeutic agent, composition, and/or formulation) that elicits a desired biological response. In some embodiments, a therapeutically effective amount of a substance is an amount that is sufficient, when administered as part of a dosing regimen to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some embodiments, a “therapeutically effective amount” is at least a minimal amount of a compound, or composition containing a compound, which is sufficient for treating one or more symptoms of a disorder or condition associated with MK2.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the disclosure. Unless otherwise stated, all tautomeric forms of the compounds described herein are within the scope of the disclosure. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this disclosure. Such compounds are useful, for example, as analytical tools, as probes in biological assays, or as therapeutic agents in accordance with the present disclosure. In certain embodiments, a warhead moiety, Ring A(R²)(R³), of a provided compound comprises one or more deuterium atoms.

Combinations of substituents and variables envisioned by this disclosure are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof. Inhibition of activity of a protein kinase, for example, MK2 or a mutant thereof, in a biological sample is useful for a variety of purposes that are known to one of skill in the art. Examples of such purposes include, but are not limited to, blood transfusion, organ transplantation, biological specimen storage, and biological assays.

The term “subject”, as used herein, means a mammal and includes human and animal subjects, such as domestic animals (e.g., horses, dogs, cats, etc.). It will be appreciated that the term “subject” is sometimes used synonymously with “patient.”

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. The amount of compounds of the present disclosure that may be combined with the carrier materials to produce a composition in a single dosage form will vary depending upon the host treated, the particular mode of administration, etc. Preferably, provided compositions are formulated so that a dosage of between 0.01 to about 100 mg/kg, or about 0.1 mg/kg to about 50 mg/kg, and preferably from about 1 mg/kg to about 25 mg/kg, of subject body weight/day of the inhibitor can be administered to a patient receiving these compositions to obtain the desired therapeutic effect. The amount of a compound of the present disclosure in the composition will also depend upon the particular compound in the composition.

As used herein, a “therapeutically effective amount” means an amount of a substance (e.g., a therapeutic agent, composition, and/or formulation) that elicits a desired biological response. In some embodiments, a therapeutically effective amount of a substance is an amount that is sufficient, when administered as part of a dosing regimen to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of a provided compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some embodiments, a “therapeutically effective amount” is at least a minimal amount of a provided compound, or composition containing a provided compound, which is sufficient for treating one or more symptoms of an MK2-mediated disease or disorder.

The terms “treat” or “treating,” as used herein, refers to partially or completely alleviating, inhibiting, delaying onset of, preventing, ameliorating and/or relieving a disorder or condition, or one or more symptoms of the disorder or condition. In some embodiments, treatment may be administered after one or more symptoms have developed. In some embodiments, the term “treating” includes preventing or halting the progression of a disease or disorder. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence. Thus, in some embodiments, the term “treating” includes preventing relapse or recurrence of a disease or disorder.

The expression “unit dosage form” as used herein refers to a physically discrete unit of a provided compound and/or compositions thereof appropriate for the subject to be treated. It will be understood, however, that the total daily usage of the active agent (i.e., compounds and compositions of the present disclosure) will be decided by the attending physician within the scope of sound medical judgment. The specific effective dose level for any particular subject (i.e., patient) or organism will depend upon a variety of factors including the disorder being treated and the severity of the disorder; activity of specific active agent employed; specific composition employed; age, body weight, general health, sex and diet of the subject; time of administration, route of administration, and rate of excretion of the specific active agent employed; duration of the treatment; and like factors well known in the medical arts.

As used herein, the term “inhibitor” is defined as a compound that binds to and/or inhibits the target protein kinase, MK2, with measurable affinity. In certain embodiments, an inhibitor has an IC₅₀ and/or binding constant of less than about 50 μM, less than about 1 μM, less than about 500 nM, less than about 100 nM, or less than about 10 nM.

The terms “measurable affinity” and “measurably inhibit,” as used herein, means a measurable change in MK2 activity between a sample comprising a compound of the present disclosure, or composition thereof, and MK2, and an equivalent sample comprising MK2, in the absence of said compound, or composition thereof.

As used herein, the term “irreversible” or “irreversible inhibitor” refers to an inhibitor (i.e. a compound) that is able to be covalently bonded to a kinase in a substantially non-reversible manner. That is, whereas a reversible inhibitor is able to bind to (but is generally unable to form a covalent bond with) a kinase, and therefore can become dissociated from the kinase, an irreversible inhibitor will remain substantially bound to a kinase once covalent bond formation has occurred. Irreversible inhibitors usually display time dependency, whereby the degree of inhibition increases with the time with which the inhibitor is in contact with the enzyme. In certain embodiments, an irreversible inhibitor will remain substantially bound to a kinase once covalent bond formation has occurred and will remain bound for a time period that is longer than the life of the protein.

Methods for identifying if a compound is acting as an irreversible inhibitor are known to one of ordinary skill in the art. Such methods include, but are not limited to, enzyme kinetic analysis of the inhibition profile of the compound with a kinase, the use of mass spectrometry of the protein drug target modified in the presence of the inhibitor compound, discontinuous exposure, also known as “washout,” experiments, and the use of labeling, such as radiolabelled inhibitor, to show covalent modification of the enzyme, as well as other methods known to one of skill in the art.

### DETAILED DESCRIPTION OF CERTAIN EMBODIMENTS OF THE INVENTION

### MK2

MAP kinase-activated protein kinase 2 (“MK2”) is an enzyme that is encoded by the human MAPKAPK2 gene. The MK2 enzyme is a serine/threonine (Ser/Thr) protein kinase that is regulated through direct phosphorylation by p38 MAP kinase.

MK2 is a multi-domain protein consisting of an N-terminal proline-rich domain, a catalytic domain, an autoinhibitory domain and at the C-terminus a nuclear export signal (NES) and nuclear localization signal (NLS). Two isoforms of human MK2 have been characterized. One isoform consists of 400 amino acids and the other isoform consists of 370 residues. Without wishing to be bound by theory, there is evidence that alternative translation of the same mRNA results in the different isoforms of MK2. For example, Trulley et al. report that one isoform results from an alternative CUG translation initiation start site located in the 5′ UTR of mRNA. Cell Rep. 2019 Jun. 4; 27(10):2859-2870 e6.

MK2 is known to be involved in many cellular processes including stress and inflammatory responses, nuclear export, gene expression regulation and cell proliferation. Indeed, MK2 regulates, by a post-transcriptional mechanism, biosynthesis of tumor necrosis factor alpha (TNF-α) that is overproduced in inflammatory diseases such as rheumatoid arthritis and inflammatory bowel disease. See Natesan et al., J. Med. Chem. 2012, 55, 2035-2047.

MK2 inhibitors prevent or block phosphorylation of heat shock protein 27 (Hsp27). Inhibition of Hsp27 phosphorylation occurs by inhibiting the formation of the p38 kinase-MK2-Hsp27 signaling complex. Phosphorylation of Hsp27 is the penultimate event in a complex signaling cascade that occurs in response to extracellular stimuli. See Zheng et al., The Journal of Biological Chemistry, vol. 281, no. 48, 37215-37226, Dec. 1, 2006. Hsp27 usually exists as oligomers and plays a role in regulation of many cellular functions such as inhibition of the death receptor-mediated apoptosis, promotion of proper refolding of denatured proteins by acting as a molecular chaperone, and regulation of cytoskeleton. The presence of MK2 is a necessary condition for the formation of p38 kinase-MK2-Hsp27 signaling complex in cells. See Zheng et al., The Journal of Biological Chemistry, vol. 281, no. 48, 37215-37226, Dec. 1, 2006.

Evidence suggests that many signaling proteins form multimeric complexes. See Zheng et al., The Journal of Biological Chemistry, vol. 281, no. 48, 37215-37226, Dec. 1, 2006. One such complex is the Hsp27/Akt (a serine/threonine kinase) dimer, which forms in the cytoplasm of a cell. Another complex is formed between MK2 and p38. See Ben-Levy et al., Current Biology 1998, 8:1049-1057; Natesan et al., J. Med. Chem. 2012, 55, 2035-2047; Zheng et al., The Journal of Biological Chemistry, vol. 281, no. 48, 37215-37226, Dec. 1, 2006.

In unstimulated conditions, inactive p38 and unphosphorylated MK2 form such dimer in the nucleus of a cell. Upon activation, p38 phosphorylates MK2, thereby inducing a conformational change of the autoinhibitory domain of MK2 and exposing the active site for substrate binding. Once MK2 is phosphorylated, the p38-MK2 dimer is translocated to the cytoplasm, where it forms a quaternary complex with the Hsp27-Akt dimer. See Zheng et al., The Journal of Biological Chemistry, vol. 281, no. 48, 37215-37226, Dec. 1, 2006. Hsp27 is then phosphorylated by MK2, resulting in degradation of the quaternary complex and the release of p-Hsp27 monomers and dimers. Because inhibition of MK2 blocks phosphorylation of Hsp27, without wishing to be bound by theory, it is believed that inhibition of MK2 prevents degradation of the p38-MK2-Akt-Hsp27 quaternary complex, thereby altering downstream effects. Consequent to the inhibition of quaternary complex degradation, the amount of quaternary complex would thereby increase. Moreover, the equilibrium of p38 and MK2 between the cytoplasm and nucleus would be shifted towards the cytoplasm.

Interestingly, transport of the MK2/p38 complex out of the nucleus does not require catalytically active MK2, as the active site mutant, Asp207Ala, is still transported to the cytoplasm. Phosphorylation of human MK2 by p38 on residues T222, S272 and T334 is thought to activate the enzyme by inducing a conformational change of the autoinhibitory domain thus exposing the active site for substrate binding. Mutations of two autoinhibitory domain residues W332A and K326E in murine MK2 demonstrate an increase in basal activity and a C-terminal deletion of the autoinhibitory domain renders the enzyme constitutively active, providing additional evidence to the role of this domain in inhibition of MK2 activity.

### COVID-19

COVID-19 is an acute respiratory disease caused by infection with the coronavirus SARS-CoV-2. A feature of SARS-CoV-2 infection is that, while the majority of patients experience relatively mild symptoms, a small proportion will develop a life threatening illness characterized by a myeloid cell driven hyper-inflammatory response leading to life-threatening hypoxia and severe acute respiratory distress syndrome (ARDS) that is the principal cause of mortality. See Huang, et al. Lancet 2020, 395, 497-506; Guan, et al. N. Engl. J. Med. 2020, 382, 1708-1720. Such inflammatory response occurs 7-10 days after the initial appearance of symptoms, and may present as a benign initial respiratory oxygen dependence that quickly requires substantial ventilatory support (over hours). ARDS is more common in patients who are older, male, and those with pre-existing co-morbidities, particularly cardiovascular and cerebrovascular disease and diabetes. ARDS occurs in 3-30% of patients and is severe enough to require ICU admission and ventilatory support in 6-20% of patients. Once a patient requires intubation, mortality rates can be up to 50%. In SARS-CoV-2 disease (e.g., COVID-19), the lung pathology has been attributed to a similar myeloid cell inflammatory cell infiltrate. Cellular analysis by single cell RNA-sequencing of bronchoalveolar lavage (BAL), in 6 patients, including 3 with mild disease and 3 with severe disease, showed a marked increase in abnormal monocyte-derived macrophage cell infiltrate. Lao, et al. Nature Medicine 2020, 26, 842-844. Analysis of patient plasma in 41 patients shows increased levels of IL-1β, IL-1Rα, IL-7, IL-8, IL-9, IL-10, basic FGF (bFGF), GCSF, GM-CSF, IFNγ, IP10, MCP1, MIP1A, MIP1B, PDGF, TNF-α, and VEGF in SARS-CoV-2 patients as compared to healthy controls. Huang, et al. Lancet 2020, 395, 497-506. Moreover, SARS-CoV-2 patients admitted to ICU showed higher plasma levels of IL-2, IL-7, IL-10, GMCSF, IL-6, IP10, MCP1, MIP1A, and TNF-α than non-ICU patients. Huang, et al. Lancet 2020, 395, 497-506.

Unfortunately, the pathogenesis of COVID-19 still remains unclear, and there are currently no FDA-approved therapies. Therefore, there remains an unmet need for therapies that lessen the severity of symptoms associated with a coronavirus (e.g., SARS-CoV-2), reduce the incidence of infection of a coronavirus (e.g., SARS-CoV-2) in a population, and/or improve overall clinical outcomes in patients susceptible to or infected with a coronavirus (e.g., SARS-CoV-2).

In some embodiments, the present disclosure provides recognition that inhibition of activity of MK2, or a mutant thereof, is useful in treating a respiratory disease or disorder associated with SARS-CoV-2 (e.g., COVID-19).

### Role of MK2 in COVID-19

One of the hallmarks of COVID-19 is a “cytokine storm” and resulting hyperinflammatory state, which contribute to the severity of COVID-19. In multiple studies, systemic and local elevations of cytokines and chemokines, including IL-1β, TNF-α, IL-6, IL-10, GM-CSF, and MCP-1, have been reported in patients with COVID-19. See Skevaki, et al. Journal of Infection 2020 Jun. 21; S0163-4453(20)30420-5; Hoiland, Br. J. Haemetol. 2020; 10.1111/bjh.16961. In particular, IL-6 and IL-10 have been found to predict disease severity. See Han, Emerging Microbes & Infections 2020, 9(1):1123-1130. Without wishing to be bound by any particular theory, inhibition of the cytokine storm may be beneficial in the treatment of COVID-19.

When activated by extracellular stress signals, MK2 phosphorylation of tristetraprolin (TTP) results in stabilization of inflammatory cytokine and chemokine mRNAs, leading to their increased production. When MK2 activity is inhibited, cytokine and chemokine mRNAs are destabilized, resulting in their reduced production. Indeed, MK2 knockout animals are protected in a variety of inflammatory models, including sepsis and lung injury. See Kotlyarov, et al. Nature Cell Biology 1999, 1, 94-97; Wu, et al. Am. J. Physiol. Lung Cell Mol. Physiol. 2018, 315, L371-L381. Without wishing to be bound by any particular theory, because inhibition of MK2 has been shown to inhibit the production of cytokines and chemokines in vitro and/or in vivo, MK2 inhibitors represent a potential treatment for the cytokine storm, hyperinflammation, and tissue injury associated with severe COVID-19.

Without wishing to be bound by any particular theory, in addition to its anti-inflammatory effects, MK2 inhibition may modulate both the direct cytopathic effects of SARS-CoV2 and/or the viral lifecycle, expanding the potential benefits of MK2 inhibitors in treating COVID-19. As discussed above, MK2 is a component of the p38 pathway, which has been shown to be activated by SARS-CoV infection, the coronavirus that causes SARS and is highly related to SARS-CoV-2. See Wang, et al. Eur. J. Microbiol. Infect. Dis. 2020, in press; Kopecky-Bromberg, et al. Journal of Virology 2006, 80, 785-793; Mizutani, et al. Biochem. and Biophys. Res. Comm. 2004, 319, 1228-1234; Fung, et al. Viruses 2016, 8, 184-199. In these studies, SARS-CoV infection-triggered p38 signaling, and resulted in activation of MK2 and the downstream MK2 target Hsp27, demonstrating that MK2 is hyperactive in SARS-CoV infected cells. Administration of a p38 inhibitor to cells infected with SARS-CoV inhibited phosphorylation of Hsp27, which is a direct downstream target of MK2 activity. See Mizutani, et al. Biochem. and Biophys. Res. Comm. 2004. Since Hsp27 is a direct substrate of MK2, administration of an MK2 inhibitor will more directly impair Hsp27 phosphorylation than p38 inhibition. In addition, infection with SARS-CoV or transfection of the SARS-CoV nucleocapsid protein has been reported to induce both apoptosis and actin cytoskeleton changes at least in part via the p38/MK2/Hsp27 pathway in in vitro cell culture models. See Padhan, et al. Journal of General Virology 2008, 89, 1960-1969; Surjit, et al. Biochem. J. 2004, 383, 13-18. Furthermore, in experiments involving a different coronavirus, transmissible gastroenteritis virus (TGEV), a p38 inhibitor demonstrated a reduction in viral titers in infected cells. See Dong, et al. Antiviral Research 2020, 173, 104651. Likewise, following infection with the murine coronavirus mouse hepatitis virus (MHV), p38 pathway activation was also demonstrated. See Banerjee, et al. Journal of Virology 2002, 76, 5937-5948. Moreover, a p38 inhibitor suppressed both inflammatory cytokine production and viral titers in this system. See Banerjee, et al. Journal of Virology 2002, 76, 5937-5948. Other inflammatory viruses that causes severe respiratory infections, such as influenza strains, also activate this pathway. See Borgeling, et al. J. of Biol. Chem. 2014, 289, 13-27. Without wishing to be bound by any particular theory, these results support a potential role for MK2 inhibition in COVID-19 treatment, e.g., by preventing both apoptosis/cytopathic changes and viral proliferation in infected cells.

A possible complication of SARS-CoV2 infection and COVID-19 disease is pulmonary fibrosis. See Lechowicz, et al. J. of Clin. Med. 2020, 9, 1917. Without wishing to be bound by any particular theory, it is believed that pulmonary fibrosis may be secondary to either direct cytopathic effects, chronic inflammation, or both, leading to lung epithelial damage and fibroblast activation. In murine models of pulmonary fibrosis, MK2 deletion or inhibition resulted in a reduction in fibroblast invasiveness, and a fibroblast specific MK2 deletion attenuated lung fibrosis in a murine model of pulmonary fibrosis. See Liang, et al. Am. J. Respir. Cell Mol. Biol. 2019, 60, 41-48. Without wishing to be bound by any particular theory, these observations suggest that MK2 inhibition may treat, lessen, or prevent the pulmonary fibrosis arising from COVID-19.

Furthermore, patients with cardiovascular disease (CVD) appear to be at higher risk of developing severe COVID-19, and cardiovascular complications in COVID-19 are frequent. See Lechowicz, et al. J. of Clin. Med. 2020, 9, 1917. Patients with CVD demonstrate elevated circulating angiotensin II levels, which can trigger p38 signaling and may promote the coronavirus life cycle. In addition, without wishing to be bound by any particular theory, direct infection of cardiomyocytes is also expected to increase p38 pathway activation, inflammation, and cytopathic effects. Further, pro-thrombotic complications are noted in COVID-19 patients. For example, the p38/MK2/Hsp27 pathway has been demonstrated to play a role in platelet activation. See Shi, et al. Arterioscler. Thromb. Vasc. Biol. 2017, 37, e185-e196; Polanowska-Grabowska, et al. Platelets 2000, 11, 6-22. Without wishing to be bound by any particular theory, it is believed that MK2 inhibition may be of particular utility in patients with COVID-19 and cardiovascular risk factors or cardiovascular/thrombotic complications.

### Compounds of Formula I

In some embodiments, compounds of the present disclosure include those of the formulae described herein, or a pharmaceutically acceptable salt thereof, wherein each variable is as defined and described herein. In some embodiments, the present disclosure provides compounds of formula I:

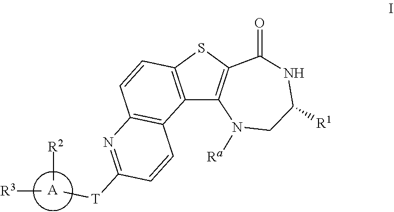

or a pharmaceutically acceptable salt thereof, wherein:
Ring A is phenyl, a 5-6 membered monocyclic heteroaryl ring having 1-3 nitrogen atoms, or an 8-14 membered bicyclic heteroaryl ring having 1-4 heteroatoms independently selected from nitrogen, oxygen, and sulfur;
T is a bivalent moiety selected from —N(R)—, —O—, —S—, —S(O)—, —SO₂—, —C(S)—, —Si(R⁴)₂—, —P(R⁵)—, —P(O)₂—, or a bivalent saturated straight or branched 1-3 membered hydrocarbon chain, wherein the hydrocarbon chain is optionally substituted with oxo or —OR;
each R is independently hydrogen or an optionally substituted C₁₋₆ aliphatic, or:
two R groups on the same nitrogen are taken together with the nitrogen to form a 3-7 membered saturated or partially unsaturated heterocyclic ring having 1-3 heteroatoms selected from nitrogen, oxygen, or sulfur;
R^{a} is hydrogen or an optionally substituted C₁-6 aliphatic;
R¹ is —R or —(CH₂)ₚR^{x};
p is 0, 1, 2, or 3;
R^{x} is —CN, —NO₂, halogen, —OR, —SR, —N(R)₂, —C(O)N(R)₂, —C(O)OR, —C(O)R, —N(R)C(O)R, —SO₂N(R)₂, or —N(R)SO₂;
R² is halogen, —CN, —SR^{y}, —S(O)R^{y}, —SO₂R^{y}, —OSO₂R^{y}, —OC(O)R^{y}, or —OP(O)₂OR^{y};
each R^{y} is independently selected from optionally substituted C₁-6 aliphatic or optionally substituted phenyl;
R³ is hydrogen, optionally substituted C₁₋₆ aliphatic, —CN, —NO₂, halogen, —OR, —N(R)₂, —C(O)N(R)₂, —C(O)OR, -Cy, —C(O)N(R)-Cy, —C(O)-Cy, —O-Cy, —O—(CH₂)ₙ-Cy, —(CH₂)ₙ—O-Cy, —(CH₂)ₘN(R)₂, —(CH₂)ₘOR, —N(R)-Cy, —N(R)—(CH₂)ₙ-Cy, —(CH₂)ₙ—N(R)-Cy, or —(CH₂)ₘ-Cy;
each R⁴ is independently hydrogen, —OR, C₁₋₆ aliphatic, phenyl, or a 5-6 membered heteroaryl ring having 1-3 heteroatoms independently selected from nitrogen, oxygen, and sulfur;
each R⁵ is independently —OR, C₁₋₆ aliphatic, phenyl, or a 5-6 membered heteroaryl ring having 1-3 heteroatoms independently selected from nitrogen, oxygen, and sulfur;
each of m and n is independently 0-4; and
each Cy is independently an optionally substituted ring selected from a 3-9 membered saturated or partially unsaturated monocyclic carbocyclic ring, a 3-9 membered saturated or partially unsaturated monocyclic heterocyclic ring having 1-3 heteroatoms independently selected from nitrogen, oxygen, and sulfur, phenyl, a 5-6 membered heteroaryl ring having 1-3 heteroatoms independently selected from nitrogen, oxygen, and sulfur, a 7-12 membered saturated or partially unsaturated fused or bridged bicyclic carbocyclic ring, or a 6-12 membered saturated or partially unsaturated fused or bridged bicyclic heterocyclic ring having 1-3 heteroatoms independently selected from nitrogen, oxygen, and sulfur.

As defined generally above and discussed throughout, each R^{∘} is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur; wherein R^{∘} may be substituted by halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●}; or two independent occurrences of R^{∘} may be optionally taken together with their intervening atom(s), form a 3-12-membered saturated, partially unsaturated, or aryl mono- or bicyclic ring having 0-4 heteroatoms independently selected from nitrogen, oxygen, or sulfur. In some such embodiments, each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As defined generally above, T is a bivalent moiety selected from —N(R)—, —O—, —S—, —S(O)—, —SO₂—, —C(S)—, —Si(R⁴)₂—, —P(R⁵)—, —P(O)₂—, or a bivalent saturated straight or branched 1-3 membered hydrocarbon chain, wherein the hydrocarbon chain is optionally substituted with oxo or —OR, wherein each R is independently hydrogen or an optionally substituted C₁₋₆ aliphatic. In some embodiments, T is —N(R)—, —O—, or —S—. In some embodiments, T is —NH—. In other embodiments, T is —O—. In other embodiments, T is —S—. In some embodiments, T is —N(R)— wherein R is optionally substituted C₁₋₆ aliphatic. In some embodiments, T is —N(CH₃)—. In some embodiments, T is —N(R)— wherein R is C₁₋₆ aliphatic optionally substituted with —(CH₂)₀₋₄N(R^{∘})₂ or —(CH₂)₀₋₄OR^{∘}. In some such embodiments, R^{∘} is as defined above and described herein. In some embodiments, T is —N(R)— wherein R is C₁₋₆ aliphatic optionally substituted with —(CH₂)₀₋₄N(R^{∘})₂ or —(CH₂)₀₋₄OR^{∘}, wherein R^{∘} is hydrogen or C₁₋₆ aliphatic. In some embodiments, T is —N(CH₂CH₂N(R^{∘})₂)— or —N(CH₂CH₂OR^{∘})—, wherein R^{∘} is hydrogen or C₁₋₆ aliphatic. In certain embodiments, T is selected from the T moieties present on the compounds depicted in Table 1, below.

In some embodiments, T is a bivalent moiety selected from —N(R)—, —O—, —S—, —S(O)—, —SO₂—, —C(S)—, —Si(R⁴)₂—, —P(R⁵)—, —P(O)₂—, a bivalent 3-7 membered cycloalkylene, or a bivalent saturated straight or branched 1-3 membered hydrocarbon chain, wherein the hydrocarbon chain is optionally substituted with halogen, —R, deuterium, oxo, or —OR, wherein each R is independently hydrogen or an optionally substituted C₁₋₆ aliphatic. In some embodiments, T is a bivalent 3-7 membered cycloalkylene, or a bivalent saturated straight or branched 1-3 membered hydrocarbon chain, wherein the hydrocarbon chain is optionally substituted with halogen, —R, deuterium, oxo, or —OR, wherein each R is independently hydrogen or an optionally substituted C₁₋₆ aliphatic. In some embodiments, T is a bivalent 3-7 membered cycloalkylene. In some embodiments, T is cyclopropylene. In some embodiments, T is 1,1-cyclopropylene. In some embodiments, T is a bivalent saturated straight or branched 1-3 membered hydrocarbon chain, wherein the hydrocarbon chain is optionally substituted with halogen, —R, deuterium, oxo, or —OR, wherein each R is independently hydrogen or an optionally substituted C₁₋₆ aliphatic. In some embodiments, T is —CF₂—, —C(Me)₂-, or —CD₂—.

As defined generally above, R^{a} is hydrogen or an optionally substituted C₁₋₆ aliphatic. In some embodiments, R^{a} is hydrogen. In some embodiments, R^{a} is an optionally substituted C₁₋₆ aliphatic. In some embodiments, R^{a} is methyl.

As defined generally above, R¹ is —R or —(CH₂)ₚR^{x}, wherein p is 0, 1, 2, or 3, and R^{x} is —CN, —NO₂, halogen, —OR, —SR, —N(R)₂, —C(O)N(R)₂, —C(O)OR, —C(O)R, —N(R)C(O)R, —SO₂N(R)₂, or —N(R)SO₂. In certain embodiments, R¹ is —R, —CH₂OR, or —CH₂N(R)₂.

In some embodiments, R¹ is —R, wherein —R is optionally substituted C₁₋₆ aliphatic. In some embodiments, R¹ is methyl. In some embodiments, R¹ is —CH₂R^{x}, wherein R^{x} is —OR or —N(R)₂. In certain embodiments, R¹ is —CH₂OCH₃. In some embodiments, R¹ is —CH₂NH₂. In some embodiments, R¹ is —CH₂NHCH₃. In some embodiments, R¹ is —CH₂N(CH₃)₂. In certain embodiments, R¹ is —CH₂OH. In certain embodiments, R¹ is selected from the R¹ moieties present on the compounds depicted in Table 1, below.

As defined generally above, Ring A is phenyl or a 5-6 membered heteroaryl ring having 1-3 nitrogen atoms. In some embodiments, Ring A is phenyl or a 5-6 membered monocyclic heteroaryl ring having 1-3 nitrogen atoms, or a 8-14 membered bicyclic heteroaryl ring having 1-4 heteroatoms independently selected from nitrogen, oxygen, and sulfur. In some embodiments, Ring A is phenyl. In some embodiments, Ring A is a 5-6 membered heteroaryl ring having 1-3 nitrogen atoms. In some embodiments, Ring A is a 8-14 bicyclic heteroaryl ring having 1-4 heteroatoms independently selected from nitrogen, oxygen, and sulfur.

In certain embodiments, Ring A is phenyl and R³ is an electron withdrawing group. One of ordinary skill in the art would recognize that certain moieties encompassed by the definition of R³ are electron withdrawing groups. Thus, in some embodiments, Ring A is phenyl and R³ is selected from —CN, —NO₂, halogen, —C(O)N(R)₂, —C(O)OR, -Cy, —C(O)N(R)-Cy, or —C(O)-Cy. In some embodiments, Ring A is phenyl and R³ is selected from —CN, halogen, —C(O)N(R)₂, —C(O)OR, -Cy, —C(O)N(R)-Cy, or —C(O)-Cy. In certain embodiments, Ring A is phenyl and R³ is hydrogen, optionally substituted C₁₋₆ aliphatic, —CN, —NO₂, halogen, —OR, —N(R)₂, —C(O)N(R)₂, —C(O)OR, -Cy, —C(O)N(R)-Cy, —C(O)-Cy, —O-Cy, —O—(CH₂)ₙ-Cy, —(CH₂)ₙ—O-Cy, —N(R)-Cy, —N(R)—(CH₂)ₙ-Cy, —(CH₂)ₙ—N(R)-Cy, or —(CH₂)ₘ-Cy. In certain embodiments, Ring A is phenyl and R³ is hydrogen, optionally substituted C₁₋₆ aliphatic, —CN, halogen, —OR, —N(R)₂, —C(O)N(R)₂, —C(O)OR, -Cy, —C(O)N(R)-Cy, —C(O)-Cy, —O-Cy, —O—(CH₂)ₙ-Cy, —(CH₂)ₙ—O-Cy, —N(R)-Cy, —N(R)—(CH₂)ₙ-Cy, —(CH₂)ₙ—N(R)-Cy, or —(CH₂)ₘ-Cy. In certain embodiments, Ring A is phenyl and R³ is selected from —CN, —NO₂, or halogen. In certain embodiments, Ring A is phenyl and R³ is selected from —CN or halogen.

In some embodiments, Ring A is phenyl and R² is at a meta position of the phenyl ring and R³ is at an ortho position of the phenyl ring. In some embodiments, Ring A is:

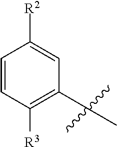

wherein R² is as defined above and herein and R³ is an electron withdrawing group and wherein the wavy line indicates the point of attachment of Ring A to T.

In some embodiments, Ring A is:

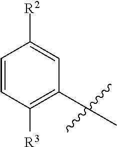

wherein R² is halogen and R³ is —CN and wherein the wavy line indicates the point of attachment of Ring A to T.

In some embodiments, Ring A is

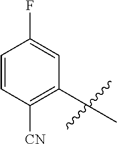 [SMILES: *c1cc(F)ccc1C#N]

wherein the wavy line indicates the point of attachment of Ring A to T.

In some embodiments, Ring A is a 5-6-membered heteroaryl ring having 1-3 nitrogen atoms. In some embodiments, Ring A is a 5-membered heteroaryl ring having 1-3 nitrogen atoms. In some embodiments, Ring A is a 6-membered heteroaryl ring having 1-3 nitrogen atoms. In some embodiments, Ring A is pyridyl. In some embodiments, Ring A is pyrimidinyl. In some embodiments, Ring A is pyridazinyl. In some embodiments, Ring A is pyrazinyl. In some embodiments Ring A is triazinyl.

In some embodiments, Ring A is a 8-14 membered bicyclic heteroaryl ring having 1-4 heteroatoms independently selected from nitrogen, oxygen, and sulfur. In some embodiments, Ring A is a 9-10 membered bicyclic heteroaryl ring having 1-4 heteroatoms independently selected from nitrogen, oxygen, and sulfur. In some embodiments, Ring A is a 9-10 membered bicyclic heteroaryl ring having 2-3 heteroatoms independently selected from nitrogen, oxygen, and sulfur. In some embodiments, Ring A is selected from:

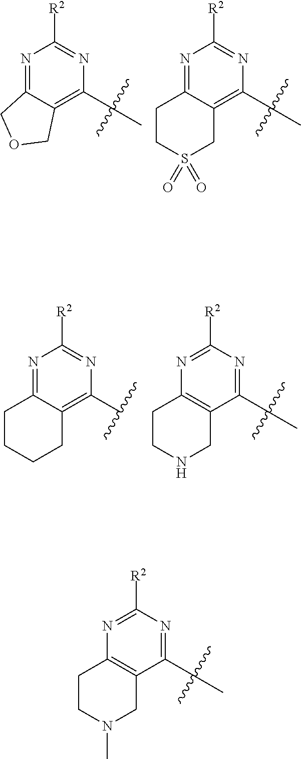

As defined generally above, R² is halogen, —CN, —SR^{y}, —S(O)R^{y}, —SO₂R^{y}, —OSO₂R^{y}, —OC(O)R^{y}, or —OP(O)₂OR^{y}, wherein each R^{y} is independently selected from optionally substituted C₁₋₆ aliphatic or optionally substituted phenyl. One of ordinary skill in the art will recognize that moieties encompassed by the definition of R² are leaving groups. Leaving groups are well known in the art, e.g., see, “Advanced Organic Chemistry,” Jerry March, 4^{th} Ed., pp. 351-357, John Wiley and Sons, N.Y. (1992). In some embodiments, R² is halogen. In some embodiments, R² is fluoro. In certain embodiments, R² is chloro. In some embodiments, R² is —SR^{y} or —SO₂R^{y}. In some embodiments, R² is —SR^{y} or —SO₂R^{y} and R^{y} is optionally substituted C₁₋₆ aliphatic. In some embodiments, R² is —SCH₃ or —SO₂CH₃. In some embodiments, R² is selected from the R² moieties present on the compounds depicted in Table 1, below.

As defined generally above, R³ is hydrogen, optionally substituted C₁₋₆ aliphatic, —CN, —NO₂, halogen, —OR, —N(R)₂, —C(O)N(R)₂, —C(O)OR, -Cy, —C(O)N(R)-Cy, —C(O)-Cy, —O-Cy, —O—(CH₂)ₙ-Cy, —(CH₂)ₙ—O-Cy, —(CH₂)ₘN(R)₂, —(CH₂)ₘOR, —N(R)-Cy, —N(R)—(CH₂)ₙ-Cy, —(CH₂)ₙ—N(R)-Cy, or —(CH₂)ₘ-Cy wherein each m and n is independently 0, 1, 2, 3, or 4, and each Cy is independently an optionally substituted ring selected from a 3-9 membered saturated or partially unsaturated carbocyclic ring or a 3-9 membered saturated or partially unsaturated heterocyclic ring having 1-3 heteroatoms independently selected from nitrogen, oxygen, and sulfur, phenyl, a 5-6 membered heteroaryl ring having 1-3 heteroatoms independently selected from nitrogen, oxygen, and sulfur, a 7-12 membered saturated or partially unsaturated fused or bridged bicyclic carbocyclic ring, or a 7-12 membered saturated or partially unsaturated fused or bridged bicyclic heterocyclic ring having 1-3 heteroatoms independently selected from nitrogen, oxygen, and sulfur.

In some embodiments, Cy is an optionally substituted 3-9 membered saturated or partially unsaturated carbocyclic ring. In some embodiments, Cy is an optionally substituted 3-7 membered saturated or partially unsaturated carbocyclic ring. In some embodiments, Cy is an optionally substituted 3-7 membered saturated carbocyclic ring. In some embodiments, Cy is an optionally substituted cyclopropyl or cyclohexyl ring.

In some embodiments, Cy is an optionally substituted 3-9 membered saturated or partially unsaturated heterocyclic ring having 1-3 heteroatoms independently selected from nitrogen, oxygen, and sulfur. In some embodiments, Cy is an optionally substituted 3-7 membered saturated or partially unsaturated heterocyclic ring having 1-3 heteroatoms independently selected from nitrogen, oxygen, and sulfur. In some embodiments, Cy is an optionally substituted 5-6 membered saturated or partially unsaturated monocyclic heterocyclic ring having 1-3 heteroatoms independently selected from nitrogen, oxygen, and sulfur.

In some embodiments, Cy is an optionally substituted 4-6 membered saturated heterocyclic ring having 1-3 heteroatoms independently selected from nitrogen, oxygen, and sulfur. In some embodiments, Cy is an optionally substituted 4-membered saturated heterocyclic ring having 1 heteroatom selected from nitrogen, oxygen, and sulfur. In some embodiments, Cy is an optionally substituted 5-membered saturated heterocyclic ring having 1-2 heteroatoms independently selected from nitrogen, oxygen, and sulfur. In some embodiments, Cy is an optionally substituted 6-membered saturated heterocyclic ring having 1-3 heteroatoms independently selected from nitrogen, oxygen, and sulfur. In certain embodiments, Cy is an optionally substituted group selected from oxetanyl, tetrahydrofuranyl, pyrrolidinyl, tetrahydropyranyl, piperidinyl, piperazinyl, and morpholinyl.

In some embodiments, Cy is an optionally substituted 3-7 membered partially unsaturated heterocyclic ring having 1-3 heteroatoms independently selected from nitrogen, oxygen, and sulfur. In some such embodiments, Cy is 3,6-dihydro-2H-pyranyl or 1,2,3,6-tetrahydropyridinyl.

In some embodiments, Cy is optionally substituted phenyl.

In some embodiments, Cy is an optionally substituted 5-6 membered heteroaryl ring having 1-3 heteroatoms independently selected from nitrogen, oxygen, and sulfur. In some embodiments, Cy is optionally substituted pyridyl.

In some embodiments, Cy is an optionally substituted 7-12 membered saturated or partially unsaturated fused or bridged bicyclic carbocyclic ring.

In some embodiments, Cy is an optionally substituted 7-12 membered saturated or partially unsaturated fused or bridged bicyclic heterocyclic ring having 1-3 heteroatoms independently selected from nitrogen, oxygen, and sulfur. In some embodiments, Cy is an optionally substituted 8-membered saturated bridged bicyclic heterocyclic ring having 1-3 heteroatoms independently selected from nitrogen, oxygen, and sulfur. In some such embodiments, Cy is (1R,5S)-3-oxa-8-azabicyclo[3.2.1]octyl (i.e., a moiety having the structure

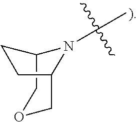 [SMILES: *N1C2CCC1COC2]

In some embodiments, a substitutable carbon atom of Cy is optionally substituted with halogen, —(CH₂)₀₋₄R^{∘}, —(CH₂)₀₋₄OR^{∘}, —(CH₂)₀₋₄N(R^{∘})₂, wherein:

R^{∘} is hydrogen or C₁₋₆ aliphatic optionally substituted with halogen or —(CH₂)₀₋₂OR^{●}, and

R^{●} is C₁₋₄ aliphatic; or:

two independent occurrences of R^{∘}, taken together with their intervening atom(s), form a 3-6 membered ring saturated ring having 0-1 heteroatoms selected from nitrogen, oxygen or sulfur.

In some embodiments, a substitutable nitrogen atom of Cy is optionally substituted with —(CH₂)₀₋₄R^{†}, wherein R^{†} is hydrogen or C₁₋₆ aliphatic.

In some embodiments, Cy is

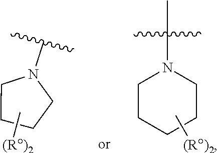

wherein each R^{∘} is C₁₋₆ aliphatic. In some embodiments, Cy is

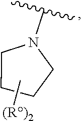

wherein each R^{∘} is C₁₋₆ aliphatic and the two occurrences of R^{∘}, taken together with their intervening atom(s), form a 3-4 membered ring saturated ring having 0-1 heteroatoms selected from nitrogen, oxygen or sulfur. In some such embodiments, Cy is 3-azabicyclo[3.1.0]hexyl (i.e., a moiety having the structure

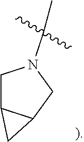 [SMILES: *N1CC2CC2C1]

In some embodiments, Cy is

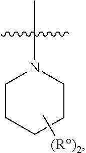

wherein each R^{∘} is C₁₋₆ aliphatic and the two occurrences of R^{∘}, taken together with their intervening atom(s), form a 3-4 membered ring saturated ring having 0-1 heteroatoms selected from nitrogen, oxygen or sulfur. In some such embodiments, Cy is 3-azabicyclo[3.1.1]heptyl (i.e., a moiety having the structure

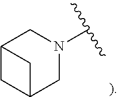 [SMILES: *N1CC2CC(C2)C1]

In certain embodiments, Cy is selected from:

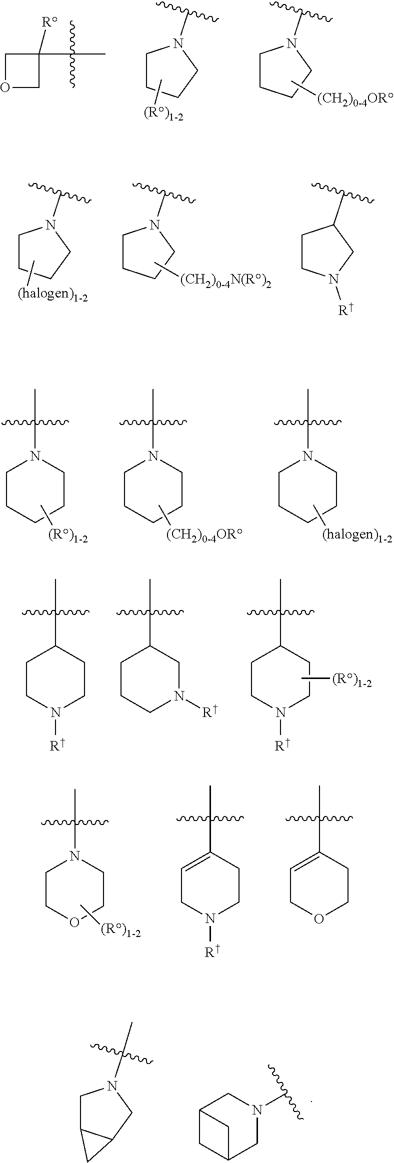

One of ordinary skill in the art would recognize that the definition of R³ includes electron-withdrawing groups (e.g., —CN, —NO₂, halogen, etc.) and solubilizing groups (e.g., —N(R)₂, -Cy, —C(O)N(R)-Cy, —C(O)-Cy, —O-Cy, —O—(CH₂)ₙ-Cy, —(CH₂)ₙ—O-Cy, —(CH₂)ₘN(R)₂, —(CH₂)ₘOR, —N(R)-Cy, —N(R)—(CH₂)ₙ-Cy, —(CH₂)ₙ—N(R)-Cy, —(CH₂)ₘ-Cy, etc.). Thus, in some embodiments, R³ is an electron-withdrawing group. In other embodiments, R³ is a solubilizing group.

In some embodiments, R³ is hydrogen. In some embodiments, R³ is optionally substituted C₁₋₆ aliphatic, —CN, —NO₂, halogen, —OR, —N(R)₂, —C(O)N(R)₂, —C(O)OR, -Cy, —C(O)N(R)-Cy, —C(O)-Cy, —O-Cy, —O—(CH₂)ₙ-Cy, —(CH₂)ₙ—O-Cy, —(CH₂)ₘN(R)₂, —(CH₂)ₘOR, —N(R)-Cy, —N(R)—(CH₂)ₙ-Cy, —(CH₂)ₙ—N(R)-Cy, or —(CH₂)ₘ-Cy. In some embodiments, R³ is optionally substituted C₁₋₆ aliphatic. In some embodiments, R³ is —(CH₂)ₘN(R)₂, —(CH₂)ₘOR, —(CH₂)ₙ—O-Cy, —(CH₂)ₘ-Cy, —(CH₂)₀₋₄N(R^{∘})₂, or —(CH₂)₀₋₄OR^{∘}. In some embodiments, R³ is C₁₋₆ aliphatic optionally substituted with —(CH₂)₀₋₄N(R^{∘})₂. In some such embodiments, R^{∘} is hydrogen or optionally substituted C₁₋₆ aliphatic. In some embodiments, R³ is C₁₋₆ aliphatic optionally substituted with —(CH₂)₀₋₄SO₂R^{∘}, —(CH₂)₀₋₄OR^{∘} or —(CH₂)₀₋₄N(R^{∘})₂, wherein R^{∘} is hydrogen or optionally substituted C₁₋₆ aliphatic. In some embodiments, R³ is —(CH₂)₀₋₄OR^{∘}. In some embodiments, R³ is —(CH₂)₀₋₄SO₂R^{∘}. In some embodiments, R³ is —(CH₂)₀₋₄N(R^{∘})₂. In some embodiments, R³ is —(CH₂)₁₋₄N(R^{∘})₂. In some embodiments, R³ is —CH₂N(R^{∘})₂. In some embodiments, R³ is —CH₂N(R^{∘})₂, —CH₂OR^{∘} or —CH₂SO₂R^{∘}. In some such embodiments, R^{∘} is C₁₋₆ aliphatic optionally substituted with —CN, halogen or —(CH₂)₀₋₂OR^{●}, wherein R^{●} is C₁₋₄ aliphatic. In some embodiments, R³ is —CH₂OH, —CH₂OCH₃, —CH₂OCHF₂, —CH₂OCH₂CHF₂, —CH₂OCH₂CH₃, —CH₂OCD₂CD₃, —CH₂OCH₂CH₂F, —CH₂OCH₂CH₂CN, —CH₂OC(CH₃)₃, —CH₂SO₂CH₃, —CH₂NHC(CH₃)₃, —CH₂N(CH₃)C(CH₃)₃, —CH₂N(CH₃)CH(CH₃)₂, —CH₂N(CH₂CH(CH₃)₂)₂, —CH₂N(CH₃)CH₂CH₂OCH₃, or —CH₂N(CH₃)CH₂CH₂OCH₂CH₃.

In some embodiments, R³ is —(CH₂)ₘN(R)₂ or —(CH₂)ₘOR. In some embodiments, R³ is —(CH₂)ₘN(R)₂. In some such embodiments, R³ is —CH₂N(R)₂. In some embodiments, R³ is —CH₂NHC(CH₃)₃. In some embodiments, R³ is —CH₂N(CH₃)C(CH₃)₃. In some embodiments, R³ is —CH₂N(CH₃)CH(CH₃)₂. In some embodiments, R³ is —CH₂N(CH₂CH(CH₃)₂)₂. In some embodiments, R³ is —CH₂N(CH₃)CH₂CH₂OCH₃. In some embodiments, R³ is —CH₂N(CH₃)CH₂CH₂OCH₂CH₃. In some such embodiments, R³ is —CH₂OR. In some embodiments, R³ is —CH₂OH. In some embodiments, R³ is —CH₂OCH₃. In some embodiments, R³ is —CH₂OCCH₂CH₃.

In some embodiments, R³ is —(CH₂)ₘ-Cy, wherein Cy is defined as above and described herein.

In some embodiments, R³ is —CH₂Cy, wherein Cy is an optionally substituted 5-6 membered saturated or partially unsaturated heterocyclic ring having 1-3 heteroatoms independently selected from nitrogen, oxygen, and sulfur.

In some embodiments, R³ is —(CH₂)ₘ-Cy, wherein Cy is an optionally substituted 7-12 membered saturated or partially unsaturated fused or bridged bicyclic heterocyclic ring having 1-3 heteroatoms independently selected from nitrogen, oxygen, and sulfur.

In some embodiments, R³ is -Cy. In some embodiments, R³ is -Cy, wherein Cy is an optionally substituted 5-6 membered heteroaryl ring having 1-3 heteroatoms independently selected from nitrogen, oxygen, and sulfur. In some embodiments, R³ is -Cy, wherein Cy is an optionally substituted 7-12 membered heteroaryl ring having 1-3 heteroatoms independently selected from nitrogen, oxygen, and sulfur. In some embodiments, R³ is -Cy, wherein Cy is as defined above and described herein.

In some embodiments, R³ is optionally substituted C₁₋₆ aliphatic selected from —CH₂OH, —CH₂OCH₃ and —CH₃.

In some embodiments, R³ is —OR, wherein R is optionally substituted C₁₋₆ aliphatic. In some embodiments, R³ is selected from —O(CH₂)₂₀CH₃, —O(CH₂)₂N(CH₃)₂, and —OCH₃.

In some embodiments, R³ is —N(R)₂, wherein R is optionally substituted C₁₋₆ aliphatic. In some embodiments, R³ is —N(CH₃)₂.

In certain embodiments, R³ is halogen, —CN, NO₂, —C(O)N(R)₂, or —C(O)OR. In some embodiments, R³ is halogen, —CN, or NO₂. In some embodiments, R³ is fluoro, chloro or bromo. In certain embodiments, R³ is —C(O)N(R)₂ or —C(O)OR, wherein each R is as defined above and described herein. In certain embodiments, R³ is selected from —C(O)NH₂, —C(O)OCH₂CH₃, and —OC(O)CH₃. In certain embodiments, R³ is selected from —C(O)NH₂, —C(O)OCH₃, —C(O)OCH₂CH₃, and —OC(O)CH₃.

In certain embodiments, R³ is -Cy, —(CH₂)ₘ-Cy, —C(O)N(R)-Cy, —C(O)-Cy, —OR, —O-Cy, or —O—(CH₂)ₙ-Cy, wherein each of R, n, m, and -Cy is as defined above and described herein.

In some embodiments, R³ is -Cy, —(CH₂)ₘ-Cy, —C(O)N(R)-Cy, —C(O)-Cy, —O-Cy, or —O—(CH₂)ₙ-Cy, wherein each -Cy is independently an optionally substituted ring selected from a 3-7 membered saturated or partially unsaturated carbocyclic ring. In some embodiments, R³ is -Cy, —(CH₂)ₘ-Cy, —C(O)N(R)-Cy, —C(O)-Cy, —O-Cy, or —O—(CH₂)ₙ-Cy, wherein each -Cy is an optionally substituted cyclopropyl ring.

In some embodiments, R³ is -Cy, —(CH₂)ₘ-Cy, —C(O)N(R)-Cy, —C(O)-Cy, —O-Cy, or —O—(CH₂)ₙ-Cy, wherein each -Cy is independently an optionally substituted ring selected from a 4-7 membered saturated or partially unsaturated heterocyclic ring having 1-3 heteroatoms independently selected from nitrogen, oxygen, and sulfur. In some embodiments, R³ is -Cy, —(CH₂)ₘ-Cy, —C(O)N(R)-Cy, —C(O)-Cy, —O-Cy, or —O—(CH₂)ₙ-Cy, wherein each -Cy is independently an optionally substituted ring selected from oxetanyl, piperidinyl, pyrrolidinyl, tetrahydrofuranyl, piperazinyl, and morpholinyl. In some embodiments, R³ is -Cy, —(CH₂)ₘ-Cy, —C(O)N(R)-Cy, —C(O)-Cy, —O-Cy, or —O—(CH₂)ₙ-Cy, wherein each -Cy is independently an optionally substituted ring selected from oxetanyl, piperidinyl, pyrrolidinyl, tetrahydrofuranyl, tetrahydropyranyl, piperazinyl, and morpholinyl. In some embodiments, R³ is —(CH₂)ₘ-Cy or C₁₋₆ aliphatic substituted by —(CH₂)₀₋₄OR^{∘}. In some embodiments, R³ is —CH₂Cy or —CH₂OR^{∘}. In some such embodiments, R^{∘} is as defined above and described herein. In some embodiments, R³ is —(CH₂)ₘ-Cy or —(CH₂)ₘOR. In some embodiments R³ is —CH₂Cy or —CH₂OR. In some embodiments R³ is —(CH₂)ₘ-Cy where Cy is optionally substituted piperidinyl.

As defined generally above, each of m and n is independently 0-4. In some embodiments, m is 1-2. In some embodiments, m is 1. In some embodiments, m is 2. In some embodiments, n is 1-2. In some embodiments, n is 1. In some embodiments, n is 2.

In some embodiments, R³ is selected from the R³ moieties present on the compounds depicted in Table 1, below.

In some embodiments, the present disclosure provides a compound of any one of formulas II, III, IV, V, or VI:

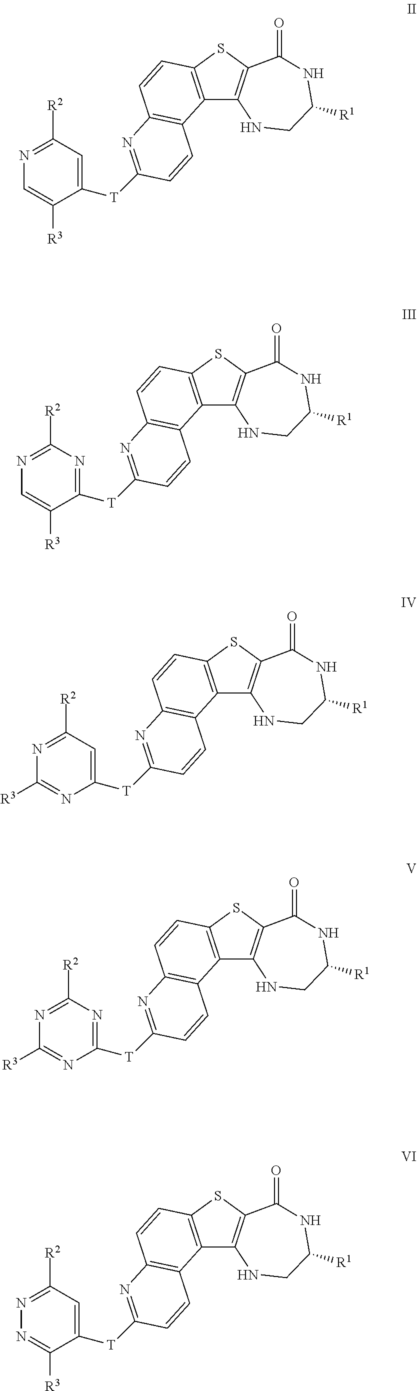

or a pharmaceutically acceptable salt thereof, wherein each of R¹, T, R², and R³ is as defined above and described herein.

In some embodiments, the present invention provides a compound of any one of formulas VII, VIII, IX, X, XI, or XII:

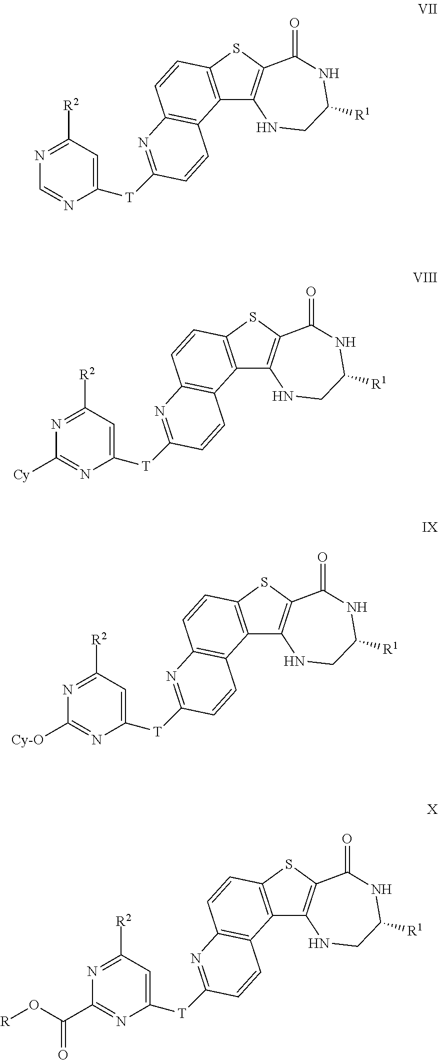

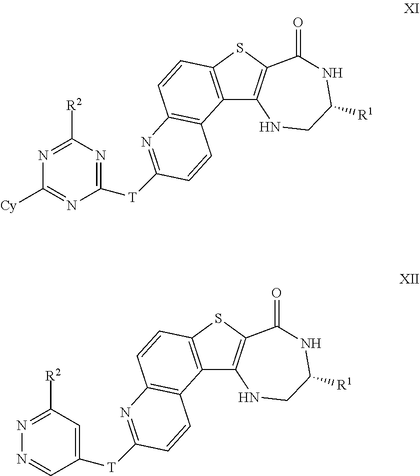

or a pharmaceutically acceptable salt thereof, wherein each of R¹, T, R², R, and -Cy is as defined above and described herein.

In some embodiments, the present invention provides a compound of any one of formulas XIII, XIV, XV, XVI, XVII, XVIII, XIX, XX, XXI, or XXII:

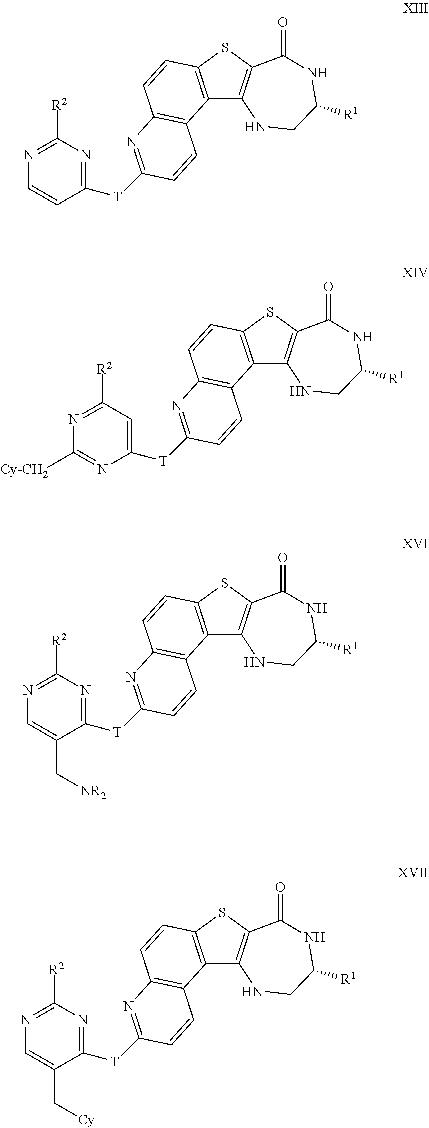

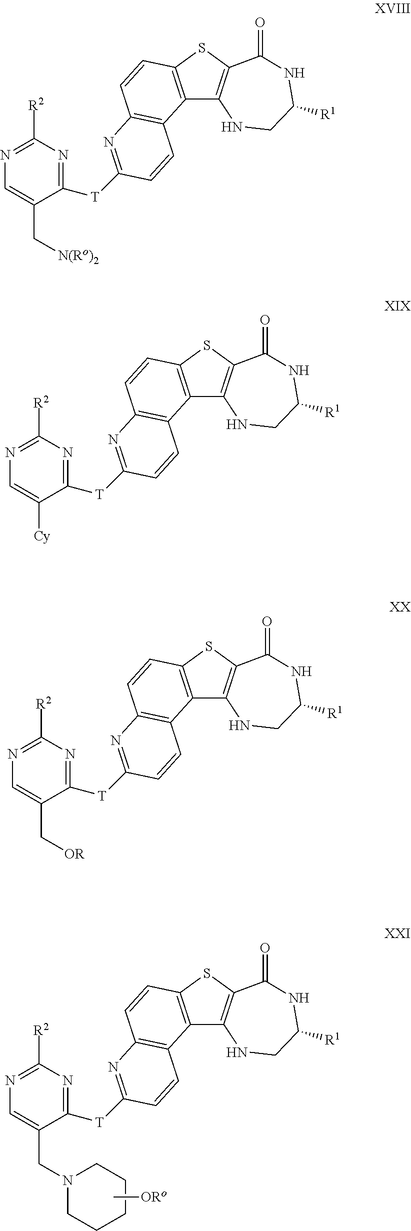

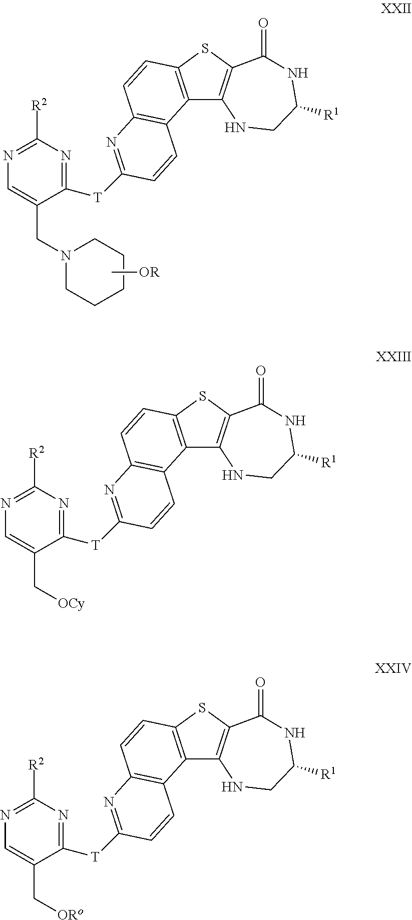

or a pharmaceutically acceptable salt thereof, wherein each of R¹, T, R², R, R^{∘}, and -Cy is as defined above and described herein.

In some embodiments, the present invention provides a compound of any one of formulas XXV or XXVI.

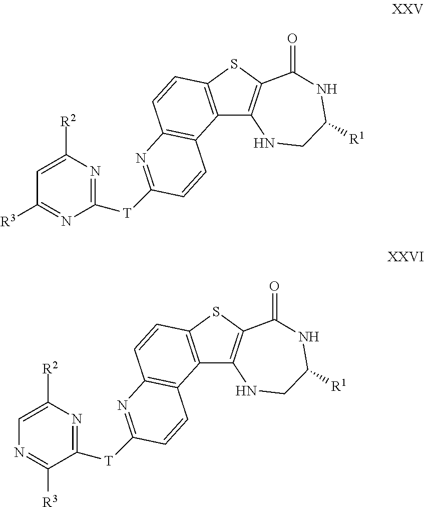

In certain embodiments, the present invention provides a compound of any one of formulas I through VI. In certain embodiments, the present invention provides a compound of any one of formulas VII through XIV. In certain embodiments, the present invention provides a compound of any one of formulas II, III, or IV. In certain embodiments, the present invention provides a compound of any one of formulas II, III, or V. In certain embodiments, the present invention provides a compound of any one of formulas II, IV, or V. In certain embodiments, the present invention provides a compound of any one of formulas III, IV, or V. In certain embodiments, the present invention provides a compound of any one of formulas III, IV, or VI. In certain embodiments, the present invention provides a compound of any one of formulas III, V, or VI. In certain embodiments, the present invention provides a compound of any one of formulas VII, VIII, IX, X, XII, XIII, or XIV. In certain embodiments, the present invention provides a compound of any one of formulas VII, VIII, IX, XII, XIII, or XIV. In certain embodiments, the present invention provides a compound of any one of formulas VII, IX, XII, XIII, or XIV. In certain embodiments, the present invention provides a compound of any one of formulas VIII, X, XI, or XIII. In certain embodiments, the present invention provides a compound of any one of formulas XX or XXI. In certain embodiments, the present invention provides a compound of any of formulas XVII or XVIII. In certain embodiments, the present invention provides a compound of any one of formulas XXV or XXVI.

In certain embodiments, the present invention provides a compound of any one of formulas I through XXVI wherein T is —O—. In certain embodiments, the present invention provides a compound of any one of formulas I through XIV wherein T is —O—. In certain embodiments, the present invention provides a compound of any one of formulas I through VI wherein T is —O—. In certain embodiments, the present invention provides a compound of any one of formulas VII through XIV wherein T is —O—. In certain embodiments, the present invention provides a compound of any one of formulas II, III, or IV wherein T is —O—. In certain embodiments, the present invention provides a compound of any one of formulas II, III, or V wherein T is —O—. In certain embodiments, the present invention provides a compound of any one of formulas II, IV, or V wherein T is —O—. In certain embodiments, the present invention provides a compound of any one of formulas III, IV, or V wherein T is —O—. In certain embodiments, the present invention provides a compound of any one of formulas III, IV, or VI wherein T is —O—. In certain embodiments, the present invention provides a compound of any one of formulas III, V, or VI wherein T is —O—. In certain embodiments, the present invention provides a compound of any one of formulas VII, VIII, IX, X, XII, XIII, or XIV wherein T is —O—. In certain embodiments, the present invention provides a compound of any one of formulas VII, VIII, IX, XII, XIII, or XIV wherein T is —O—. In certain embodiments, the present invention provides a compound of any one of formulas VII, IX, XII, XIII, or XIV wherein T is —O—. In certain embodiments, the present invention provides a compound of any one of formulas VIII, X, XI, or XIII wherein T is —O—.

In certain embodiments, the present invention provides a compound of any one of formulas I through XXVI wherein T is —NH—. In certain embodiments, the present invention provides a compound of any one of formulas I through XIV wherein T is —NH—. In certain embodiments, the present invention provides a compound of any one of formulas I through VI wherein T is —NH—. In certain embodiments, the present invention provides a compound of any one of formulas VII through XIV wherein T is —NH—. In certain embodiments, the present invention provides a compound of any one of formulas II, III, or IV wherein T is —NH—. In certain embodiments, the present invention provides a compound of any one of formulas II, III, or V wherein T is —NH—. In certain embodiments, the present invention provides a compound of any one of formulas II, IV, or V wherein T is —NH—. In certain embodiments, the present invention provides a compound of any one of formulas III, IV, or V wherein T is —NH—. In certain embodiments, the present invention provides a compound of any one of formulas III, IV, or VI wherein T is —NH—. In certain embodiments, the present invention provides a compound of any one of formulas III, V, or VI wherein T is —NH—. In certain embodiments, the present invention provides a compound of any one of formulas VII, VIII, IX, X, XII, XIII, or XIV wherein T is —NH—. In certain embodiments, the present invention provides a compound of any one of formulas VII, VIII, IX, XII, XIII, or XIV wherein T is —NH—. In certain embodiments, the present invention provides a compound of any one of formulas VII, IX, XII, XIII, or XIV wherein T is —NH—. In certain embodiments, the present invention provides a compound of any one of formulas VIII, X, XI, or XIII wherein T is —NH—.

In certain embodiments, the present invention provides a compound of any one of formulas I through XXIV wherein R² is chloro or fluoro. In certain embodiments, the present invention provides a compound of any one of formulas I through XIV wherein R² is chloro or fluoro. In certain embodiments, the present invention provides a compound of any one of formulas I through VI wherein R² is chloro or fluoro. In certain embodiments, the present invention provides a compound of any one of formulas VII through XIV wherein R² is chloro or fluoro. In certain embodiments, the present invention provides a compound of any one of formulas II, III, or IV wherein R² is chloro or fluoro. In certain embodiments, the present invention provides a compound of any one of formulas II, III, or V wherein R² is chloro or fluoro. In certain embodiments, the present invention provides a compound of any one of formulas II, IV, or V wherein R² is chloro or fluoro. In certain embodiments, the present invention provides a compound of any one of formulas III, IV, or V wherein R² is chloro or fluoro. In certain embodiments, the present invention provides a compound of any one of formulas III, IV, or VI wherein R² is chloro or fluoro. In certain embodiments, the present invention provides a compound of any one of formulas III, V, or VI wherein R² is chloro or fluoro. In certain embodiments, the present invention provides a compound of any one of formulas VII, VIII, IX, X, XII, XIII, or XIV wherein R² is chloro or fluoro. In certain embodiments, the present invention provides a compound of any one of formulas VII, VIII, IX, XII, XIII, or XIV wherein R² is chloro or fluoro. In certain embodiments, the present invention provides a compound of any one of formulas VII, IX, XII, XIII, or XIV wherein R² is chloro or fluoro. In certain embodiments, the present invention provides a compound of any one of formulas VIII, X, XI, or XIII wherein R² is chloro or fluoro.

In certain embodiments, the present invention provides a compound of formula I or III, wherein R² is halogen. In certain embodiments, the present invention provides a compound of formula I or III, wherein R² is halogen.

In certain embodiments, the present invention provides a compound of formula I or III, wherein T is —O— and R² is halogen. In certain embodiments, the present invention provides a compound of formula I or III, wherein T is —O—, R² is halogen, and R³ is —CH₂Cy. In certain embodiments, the present invention provides a compound of formula I or III, wherein T is —O—, R² is halogen, and R³ is —(CH₂)ₘCy, —(CH₂)ₙOCy, or C₁₋₆ aliphatic substituted by —(CH₂)₀₋₄N(R^{∘})₂, or —(CH₂)₀₋₄OR^{∘}. In some such embodiments, R^{∘} is as defined above and described herein.

In certain embodiments, the present invention provides a compound of formula I wherein T is —O—, R² is halogen, and R³ is —(CH₂)ₘCy, —(CH₂)ₙOCy, —(CH₂)ₘN(R)₂, or —(CH₂)ₘOR.

In certain embodiments, the present invention provides a compound of formula I or III, wherein R³ is —(CH₂)ₘCy, —(CH₂)ₙOCy, or C₁₋₆ aliphatic substituted by —(CH₂)₀₋₄N(R^{∘})₂, or —(CH₂)₀₋₄OR^{∘}. In some such embodiments, R^{∘} is as defined above and described herein. In certain embodiments, the present invention provides a compound of formula I or III, wherein R³ is —(CH₂)ₘCy, or —(CH₂)ₙOCy. In some embodiments the present invention provides a compound of formula I or III, wherein R³ is C₁₋₆ aliphatic substituted by —(CH₂)₀₋₄N(R^{∘})₂, or —(CH₂)₀₋₄OR^{∘}. In some such embodiments, R^{∘} is as defined above and described herein. In certain embodiments, the present invention provides a compound of formula I or III, wherein R³ is C₁₋₆ aliphatic substituted by —(CH₂)₀₋₄N(R^{∘})₂, or —(CH₂)₀₋₄OR^{∘}, wherein R^{∘} is hydrogen, C₁₋₆ aliphatic, or a 5-6-membered saturated, partially unsaturated, or aryl ring having 0-4 heteroatoms independently selected from nitrogen, oxygen, or sulfur, wherein R^{∘} is optionally substituted by halogen, —CN or —(CH₂)₀₋₂OR^{●}, wherein R^{●} is C₁₋₄ aliphatic. In certain embodiments, the present invention provides a compound of formula I or III, wherein R³ is —(CH₂)ₘCy or C₁₋₆ aliphatic substituted by —(CH₂)₀₋₄OR^{∘}. In some such embodiments, R^{∘} is as defined above and described herein. In certain embodiments, the present invention provides a compound of formula I or III, wherein R³ is —(CH₂)ₘCy or C₁₋₆ aliphatic substituted by —(CH₂)₀₋₄OR^{∘}, wherein R^{∘} is hydrogen, C₁₋₆ aliphatic, or a 5-6-membered saturated, partially unsaturated, or aryl ring having 0-4 heteroatoms independently selected from nitrogen, oxygen, or sulfur, wherein R^{∘} is optionally substituted by halogen, —CN or —(CH₂)₀₋₂OR^{●}, wherein R^{●} is C₁₋₄ aliphatic. In certain embodiments, the present invention provides a compound of formula I or III, wherein R³ is —(CH₂)ₘCy. In certain embodiments, the present invention provides a compound of formula I or III, wherein R³ is —(CH₂)ₙOCy. In some embodiments the present invention provides a compound of formula I or III, wherein R³ is C₁₋₆ aliphatic substituted by —(CH₂)₀₋₄N(R^{∘})₂. In some such embodiments, R^{∘} is as defined above and described herein. In some embodiments, the present invention provides a compound of formula I or III, wherein R³ is C₁₋₆ aliphatic substituted by —(CH₂)₀₋₄N(R^{∘})₂, wherein R^{∘} is hydrogen, C₁₋₆ aliphatic, or a 5-6-membered saturated, partially unsaturated, or aryl ring having 0-4 heteroatoms independently selected from nitrogen, oxygen, or sulfur, wherein R^{∘} is optionally substituted by —(CH₂)₀₋₂OR^{●}, wherein R^{●} is C₁₋₄ aliphatic. In some embodiments the present invention provides a compound of formula I or III, wherein R³ is C₁₋₆ aliphatic substituted by —(CH₂)₀₋₄OR^{∘}. In some such embodiments, R^{∘} is as defined above and described herein. In some embodiments the present invention provides a compound of formula I or III, wherein R³ is C₁₋₆ aliphatic substituted by —(CH₂)₀₋₄OR^{∘}, wherein R^{∘} is hydrogen, C₁₋₆ aliphatic, or a 5-6-membered saturated, partially unsaturated, or aryl ring having 0-4 heteroatoms independently selected from nitrogen, oxygen, or sulfur, wherein R^{∘} is optionally substituted by halogen, —CN or —(CH₂)₀₋₂OR^{●}, wherein R^{●} is C₁₋₄ aliphatic. In certain embodiments, the present invention provides a compound of formula I or III, wherein R³ is —(CH₂)ₘCy, —(CH₂)ₙOCy, or C₁₋₆ aliphatic substituted by —(CH₂)₀₋₄N(R^{∘})₂, or —(CH₂)₀₋₄OR^{∘}, wherein each R^{∘} is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur; and R^{∘} may be substituted by halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●}. In some such embodiments, R is C₁₋₄ aliphatic.

In certain embodiments, the present invention provides a compound of one of formula I, wherein R³ is —(CH₂)ₘCy, —(CH₂)ₙOCy, —(CH₂)ₘN(R)₂, or —(CH₂)ₘOR. In certain embodiments, the present invention provides a compound of formula I, wherein R³ is —(CH₂)ₘCy, or —(CH₂)ₙOCy. In some embodiments the present invention provides a compound of formula I, wherein R³ is —(CH₂)ₘN(R)₂, or —(CH₂)ₘOR. In certain embodiments, the present invention provides a compound of one of formula I wherein R³ is —(CH₂)ₘCy or —(CH₂)ₘOR. In certain embodiments, the present invention provides a compound of formula I, wherein R³ is —(CH₂)ₘCy. In certain embodiments, the present invention provides a compound of formula I, wherein R³ is —(CH₂)ₙOCy. In some embodiments the present invention provides a compound of formula I, wherein R³ is —(CH₂)ₘN(R)₂. In some embodiments the present invention provides a compound of formula I, wherein R³ is —(CH₂)ₘOR. In certain embodiments, the present invention provides a compound of one of formula I, wherein R³ is —(CH₂)ₘCy, —(CH₂)ₙOCy, —(CH₂)ₘN(R)₂, or —(CH₂)ₘOR, wherein each R is independently hydrogen or C₁₋₆ aliphatic, wherein said aliphatic or said Cy may be substituted with halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur; and R^{∘} may be substituted by halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●}. In some such embodiments, R^{●} is C₁₋₄ aliphatic.

In certain embodiments, the present invention provides a compound of formula I or III, wherein R³ is —(CH₂)ₘCy where Cy is an optionally substituted 3-9 membered saturated or partially unsaturated monocyclic heterocyclic ring having 1-3 heteroatoms independently selected from nitrogen, oxygen, and sulfur; or a 7-12 membered saturated or partially unsaturated fused or bridged bicyclic heterocyclic ring having 1-3 heteroatoms independently selected from nitrogen, oxygen, and sulfur. In certain embodiments, the present invention provides a compound of formula I or III, wherein R³ is —(CH₂)ₘCy where Cy is an optionally substituted 3-9 membered saturated or partially unsaturated monocyclic heterocyclic ring having 1-3 heteroatoms independently selected from nitrogen, oxygen, and sulfur. In certain embodiments, the present invention provides a compound of formula I or III, wherein R³ is —(CH₂)ₘCy where Cy is an optionally substituted 7-12 membered saturated fused or bridged bicyclic heterocyclic ring having 1-3 heteroatoms independently selected from nitrogen, oxygen, and sulfur. In certain embodiments, the present invention provides a compound of formula I or III, wherein R³ is —(CH₂)ₘCy where Cy is a 3-9 membered saturated or partially unsaturated monocyclic heterocyclic ring having 1-3 heteroatoms independently selected from nitrogen, oxygen, and sulfur, wherein said ring is optionally substituted with oxo, halogen; —(CH₂)₀₋₄R^{∘}; —(CH₂)₀₋₄OR^{∘}. In some such embodiments, R^{∘} is as defined above and described herein. In certain embodiments, the present invention provides a compound of formula I or III, wherein R³ is —(CH₂)ₘCy where Cy is an optionally substituted 3-9 membered saturated or partially unsaturated monocyclic heterocyclic ring having 1-3 heteroatoms independently selected from nitrogen, oxygen, and sulfur, wherein: a substitutable carbon atom on Cy is optionally substituted with halogen, —(CH₂)₀₋₄R^{∘}, or —(CH₂)₀₋₄OR^{∘}, and a substitutable nitrogen atom on Cy is optionally substituted with —R^{†}, wherein R^{∘} is hydrogen or C₁₋₆ aliphatic optionally substituted by halogen or —(CH₂)₀₋₂OR^{●}, wherein R^{●} is C₁₋₄ aliphatic, and R^{†} is C₁₋₆ aliphatic. In some such embodiments, two independent occurrences of R^{∘} may be optionally taken together with their intervening atom(s), form a 3-12-membered saturated, partially unsaturated, or aryl mono- or bicyclic ring having 0-4 heteroatoms independently selected from nitrogen, oxygen, or sulfur. In certain embodiments, the present invention provides a compound of formula I or III, wherein R³ is —CH₂Cy where Cy is an optionally substituted 3-9 membered saturated or partially unsaturated monocyclic heterocyclic ring having 1-3 heteroatoms independently selected from nitrogen, oxygen, and sulfur; or a 7-12 membered saturated or partially unsaturated fused or bridged bicyclic heterocyclic ring having 1-3 heteroatoms independently selected from nitrogen, oxygen, and sulfur. In certain embodiments, the present invention provides a compound of formula I or III, wherein R³ is —CH₂Cy where Cy is an optionally substituted 3-9 membered saturated or partially unsaturated monocyclic heterocyclic ring having 1-3 heteroatoms independently selected from nitrogen, oxygen, and sulfur. In certain embodiments, the present invention provides a compound of formula I or III, wherein R³ is —CH₂Cy where Cy is an optionally substituted 7-12 membered saturated or partially unsaturated fused or bridged bicyclic heterocyclic ring having 1-3 heteroatoms independently selected from nitrogen, oxygen, and sulfur. In certain embodiments, the present invention provides a compound of formula I or III, wherein R³ is —CH₂Cy where Cy is a 3-9 membered saturated or partially unsaturated monocyclic heterocyclic ring having 1-3 heteroatoms independently selected from nitrogen, oxygen, and sulfur, wherein said ring is optionally substituted with oxo, halogen; —(CH₂)₀₋₄R^{∘}; —(CH₂)₀₋₄OR^{∘}. In some such embodiments, R^{∘} is as defined above and described herein. In certain embodiments, the present invention provides a compound of formula I or III, wherein R³ is —CH₂Cy where Cy is an optionally substituted 3-9 membered saturated or partially unsaturated monocyclic heterocyclic ring having 1-3 heteroatoms independently selected from nitrogen, oxygen, and sulfur, wherein: a substitutable carbon atom on Cy is optionally substituted with halogen, —(CH₂)₀₋₄R^{∘}, or —(CH₂)₀₋₄OR^{∘}, and a substitutable nitrogen atom on Cy is optionally substituted with —R^{†}, wherein R^{∘} is hydrogen or C₁₋₆ aliphatic optionally substituted by halogen or —(CH₂)₀₋₂OR^{●}, wherein R^{●} is C₁₋₄ aliphatic, and R^{†} is C₁₋₆ aliphatic. In some such embodiments, two independent occurrences of R^{∘} may be optionally taken together with their intervening atom(s), form a 3-12-membered saturated, partially unsaturated, or aryl mono- or bicyclic ring having 0-4 heteroatoms independently selected from nitrogen, oxygen, or sulfur. In certain embodiments, the present invention provides a compound of formula I or III, wherein R³ is —CH₂Cy where Cy is a 7-12 membered saturated or partially unsaturated fused or bridged bicyclic heterocyclic ring having 1-3 heteroatoms independently selected from nitrogen, oxygen, and sulfur, wherein said ring is optionally substituted with oxo, halogen, —(CH₂)₀₋₄R^{∘}, —(CH₂)₀₋₄OR^{∘}. In some such embodiments, R^{∘} is as defined above and described herein. In certain embodiments, the present invention provides a compound of formula I or III, wherein R³ is —CH₂Cy where Cy is a 7-12 membered saturated or partially unsaturated fused or bridged bicyclic heterocyclic ring having 1-3 heteroatoms independently selected from nitrogen, oxygen, and sulfur, wherein: a substitutable carbon atom on Cy is optionally substituted with halogen, —(CH₂)₀₋₄R^{∘}, or —(CH₂)₀₋₄OR^{∘}, and a substitutable nitrogen atom on Cy is optionally substituted with —R^{†}, wherein R^{∘} is hydrogen or C₁₋₆ aliphatic optionally substituted by halogen or —(CH₂)₀₋₂OR^{●}, wherein R^{●} is C₁₋₄ aliphatic, and R^{†} is C₁₋₆ aliphatic. In some such embodiments, two independent occurrences of R^{∘} may be optionally taken together with their intervening atom(s), form a 3-12-membered saturated, partially unsaturated, or aryl mono- or bicyclic ring having 0-4 heteroatoms independently selected from nitrogen, oxygen, or sulfur.

In certain embodiments, the present invention provides a compound of formula I, wherein R³ is —(CH₂)ₘCy where Cy is an optionally substituted 3-9 membered saturated or partially unsaturated monocyclic heterocyclic ring having 1-3 heteroatoms independently selected from nitrogen, oxygen, and sulfur; or a 6-12 membered saturated or partially unsaturated fused or bridged bicyclic heterocyclic ring having 1-3 heteroatoms independently selected from nitrogen, oxygen, and sulfur. In certain embodiments, the present invention provides a compound of formula I, wherein R³ is —(CH₂)ₘCy where Cy is an optionally substituted 3-9 membered saturated or partially unsaturated monocyclic heterocyclic ring having 1-3 heteroatoms independently selected from nitrogen, oxygen, and sulfur. In certain embodiments, the present invention provides a compound of formula I, wherein R³ is —(CH₂)ₘCy where Cy is an optionally substituted 6-12 membered saturated or partially unsaturated fused or bridged bicyclic heterocyclic ring having 1-3 heteroatoms independently selected from nitrogen, oxygen, and sulfur. In certain embodiments, the present invention provides a compound of formula I, wherein R³ is —(CH₂)ₘCy where Cy is a 3-9 membered saturated or partially unsaturated monocyclic heterocyclic ring having 1-3 heteroatoms independently selected from nitrogen, oxygen, and sulfur, wherein said ring is optionally substituted with oxo, halogen; —(CH₂)₀₋₄R^{∘}; —(CH₂)₀₋₄OR^{∘}. In some such embodiments, R^{∘} is as defined above and described herein. In certain embodiments, the present invention provides a compound of formula I wherein R³ is —(CH₂)ₘCy where Cy is a 3-9 membered saturated or partially unsaturated monocyclic heterocyclic ring having 1-3 heteroatoms independently selected from nitrogen, oxygen, and sulfur, wherein: a substitutable carbon atom on Cy is optionally substituted with halogen, —(CH₂)₀₋₄R^{∘}, or —(CH₂)₀₋₄OR^{∘}, and a substitutable nitrogen atom on Cy is optionally substituted with —R^{†}, wherein R^{∘} is hydrogen or C₁₋₆ aliphatic optionally substituted by halogen or —(CH₂)₀₋₂OR^{●}, wherein R^{●} is C₁₋₄ aliphatic, and R^{†} is C₁₋₆ aliphatic. In some such embodiments, two independent occurrences of R^{∘} may be optionally taken together with their intervening atom(s), form a 3-12-membered saturated, partially unsaturated, or aryl mono- or bicyclic ring having 0-4 heteroatoms independently selected from nitrogen, oxygen, or sulfur. In certain embodiments, the present invention provides a compound of formula I, wherein R³ is —CH₂Cy where Cy is an optionally substituted 3-9 membered saturated or partially unsaturated monocyclic heterocyclic ring having 1-3 heteroatoms independently selected from nitrogen, oxygen, and sulfur; or a 6-12 membered saturated or partially unsaturated fused or bridged bicyclic heterocyclic ring having 1-3 heteroatoms independently selected from nitrogen, oxygen, and sulfur. In certain embodiments, the present invention provides a compound of formula I, wherein R³ is —CH₂Cy where Cy is an optionally substituted 3-9 membered saturated or partially unsaturated monocyclic heterocyclic ring having 1-3 heteroatoms independently selected from nitrogen, oxygen, and sulfur. In certain embodiments, the present invention provides a compound of formula I, wherein R³ is —CH₂Cy where Cy is an optionally substituted 6-12 membered saturated fused or bridged bicyclic heterocyclic ring having 1-3 heteroatoms independently selected from nitrogen, oxygen, and sulfur. In certain embodiments, the present invention provides a compound of formula I, wherein R³ is —CH₂Cy where Cy is a 3-9 membered saturated or partially unsaturated monocyclic heterocyclic ring having 1-3 heteroatoms independently selected from nitrogen, oxygen, and sulfur, wherein said ring is optionally substituted with oxo, halogen; —(CH₂)₀₋₄R^{∘}; —(CH₂)₀₋₄OR^{∘}. In some such embodiments, R^{∘} is as defined above and described herein. In certain embodiments, the present invention provides a compound of formula I, wherein R³ is —CH₂Cy where Cy is a 3-9 membered saturated or partially unsaturated monocyclic heterocyclic ring having 1-3 heteroatoms independently selected from nitrogen, oxygen, and sulfur, wherein: a substitutable carbon atom on Cy is optionally substituted with halogen, —(CH₂)₀₋₄R^{∘}, or —(CH₂)₀₋₄OR^{∘}, and a substitutable nitrogen atom on Cy is optionally substituted with —R^{†}, wherein R^{∘} is hydrogen or C₁₋₆ aliphatic optionally substituted by halogen or —(CH₂)₀₋₂OR^{●}, wherein R^{●} is C₁₋₄ aliphatic, and R^{†} is C₁₋₆ aliphatic. In some such embodiments, two independent occurrences of R^{∘} may be optionally taken together with their intervening atom(s), form a 3-12-membered saturated, partially unsaturated, or aryl mono- or bicyclic ring having 0-4 heteroatoms independently selected from nitrogen, oxygen, or sulfur. In certain embodiments, the present invention provides a compound of formula I, wherein R³ is —CH₂Cy where Cy is a 6-12 membered saturated fused or bridged bicyclic heterocyclic ring having 1-3 heteroatoms independently selected from nitrogen, oxygen, and sulfur, wherein said ring is optionally substituted with oxo, halogen; —(CH₂)₀₋₄R^{∘}; —(CH₂)₀₋₄OR^{∘}. In some such embodiments, R^{∘} is as defined above and described herein. In certain embodiments, the present invention provides a compound of formula I, wherein R³ is —CH₂Cy where Cy is a 6-12 membered saturated fused or bridged bicyclic heterocyclic ring having 1-3 heteroatoms independently selected from nitrogen, oxygen, and sulfur, wherein: a substitutable carbon atom on Cy is optionally substituted with halogen, —(CH₂)₀₋₄R^{∘}, or —(CH₂)₀₋₄OR^{∘}, and a substitutable nitrogen atom on Cy is optionally substituted with —R^{†}, wherein R^{∘} is hydrogen or C₁₋₆ aliphatic optionally substituted by halogen or —(CH₂)₀₋₂OR^{●}, wherein R^{●} is C₁₋₄ aliphatic, and R^{†} is C₁₋₆ aliphatic. In some such embodiments, two independent occurrences of R^{∘} may be optionally taken together with their intervening atom(s), form a 3-12-membered saturated, partially unsaturated, or aryl mono- or bicyclic ring having 0-4 heteroatoms independently selected from nitrogen, oxygen, or sulfur.

In certain embodiments, the present invention provides a compound of formula I or III, wherein R³ is —(CH₂)ₙOCy where Cy is a 3-9 membered saturated or partially unsaturated monocyclic heterocyclic ring having 1-3 heteroatoms independently selected from nitrogen, oxygen, and sulfur; or a 7-12 membered saturated or partially unsaturated fused or bridged bicyclic heterocyclic ring having 1-3 heteroatoms independently selected from nitrogen, oxygen, and sulfur. In certain embodiments, the present invention provides a compound of formula I or III, wherein R³ is —(CH₂)ₙOCy where Cy is a 3-9 membered saturated or partially unsaturated monocyclic heterocyclic ring having 1-3 heteroatoms independently selected from nitrogen, oxygen, and sulfur. In certain embodiments, the present invention provides a compound of formula I or III, wherein R³ is —(CH₂)ₙOCy where Cy is a 7-12 membered saturated or partially unsaturated fused or bridged bicyclic heterocyclic ring having 1-3 heteroatoms independently selected from nitrogen, oxygen, and sulfur. In certain embodiments, the present invention provides a compound of formula I or III, wherein R³ is —(CH₂)ₙOCy where Cy is a 3-9 membered saturated or partially unsaturated monocyclic heterocyclic ring having 1-3 heteroatoms independently selected from nitrogen, oxygen, and sulfur, wherein said ring is optionally substituted with oxo, halogen; —(CH₂)₀₋₄R^{∘}; —(CH₂)₀₋₄OR^{∘}. In some such embodiments, R^{∘} is as defined above and described herein. In certain embodiments, the present invention provides a compound of formula I or III, wherein R³ is —(CH₂)ₙOCy where Cy is a 3-9 membered saturated or partially unsaturated monocyclic heterocyclic ring having 1-3 heteroatoms independently selected from nitrogen, oxygen, and sulfur, wherein: a substitutable carbon atom on Cy is optionally substituted with halogen, —(CH₂)₀₋₄R^{∘}, or —(CH₂)₀₋₄OR^{∘}, and a substitutable nitrogen atom on Cy is optionally substituted with —R^{†}, wherein R^{∘} is hydrogen or C₁₋₆ aliphatic optionally substituted by halogen or —(CH₂)₀₋₂OR^{●}, wherein R^{●} is C₁₋₄ aliphatic, and R^{†} is C₁₋₆ aliphatic. In some such embodiments, two independent occurrences of R^{∘} may be optionally taken together with their intervening atom(s), form a 3-12-membered saturated, partially unsaturated, or aryl mono- or bicyclic ring having 0-4 heteroatoms independently selected from nitrogen, oxygen, or sulfur. In certain embodiments, the present invention provides a compound of formula I or III, wherein R³ is —(CH₂)ₙOCy where Cy is a 7-12 membered saturated or partially unsaturated fused or bridged bicyclic heterocyclic ring having 1-3 heteroatoms independently selected from nitrogen, oxygen, and sulfur, wherein said ring is optionally substituted with oxo, halogen; —(CH₂)₀₋₄R^{∘}; —(CH₂)₀₋₄OR^{∘}. In some such embodiments, R^{∘} is as defined above and described herein. In certain embodiments, the present invention provides a compound of formula I or III, wherein R³ is —(CH₂)ₙOCy where Cy is a 7-12 membered saturated or partially unsaturated fused or bridged bicyclic heterocyclic ring having 1-3 heteroatoms independently selected from nitrogen, oxygen, and sulfur, wherein: a substitutable carbon atom on Cy is optionally substituted with halogen, —(CH₂)₀₋₄R^{∘}, or —(CH₂)₀₋₄OR^{∘}, and a substitutable nitrogen atom on Cy is optionally substituted with —R^{†}, wherein R^{∘} is hydrogen or C₁₋₆ aliphatic optionally substituted by halogen or —(CH₂)₀₋₂OR^{●}, wherein R^{●} is C₁₋₄ aliphatic, and R^{†} is C₁₋₆ aliphatic. In some such embodiments, two independent occurrences of R^{∘} may be optionally taken together with their intervening atom(s), form a 3-12-membered saturated, partially unsaturated, or aryl mono- or bicyclic ring having 0-4 heteroatoms independently selected from nitrogen, oxygen, or sulfur.

In certain embodiments, the present invention provides a compound of formula I, wherein R³ is —(CH₂)ₙOCy where Cy is a 3-9 membered saturated or partially unsaturated monocyclic heterocyclic ring having 1-3 heteroatoms independently selected from nitrogen, oxygen, and sulfur; or a 6-12 membered saturated or partially unsaturated fused or bridged bicyclic heterocyclic ring having 1-3 heteroatoms independently selected from nitrogen, oxygen, and sulfur. In certain embodiments, the present invention provides a compound of formula I, wherein R³ is —(CH₂)ₙOCy where Cy is a 3-9 membered saturated or partially unsaturated monocyclic heterocyclic ring having 1-3 heteroatoms independently selected from nitrogen, oxygen, and sulfur. In certain embodiments, the present invention provides a compound of formula I, wherein R³ is —(CH₂)ₙOCy where Cy is a 6-12 membered saturated or partially unsaturated fused or bridged bicyclic heterocyclic ring having 1-3 heteroatoms independently selected from nitrogen, oxygen, and sulfur. In certain embodiments, the present invention provides a compound of formula I, wherein R³ is —(CH₂)ₙOCy where Cy is a 3-9 membered saturated or partially unsaturated monocyclic heterocyclic ring having 1-3 heteroatoms independently selected from nitrogen, oxygen, and sulfur; or a 6-12 membered saturated or partially unsaturated fused or bridged bicyclic heterocyclic ring having 1-3 heteroatoms independently selected from nitrogen, oxygen, and sulfur, wherein said ring is optionally substituted with oxo, halogen; —(CH₂)₀₋₄R^{∘}; —(CH₂)₀₋₄OR^{∘}. In some such embodiments, R^{∘} is as defined above and described herein. In certain embodiments, the present invention provides a compound of formula I, wherein R³ is —(CH₂)ₙOCy where Cy is a 3-9 membered saturated or partially unsaturated monocyclic heterocyclic ring having 1-3 heteroatoms independently selected from nitrogen, oxygen, and sulfur; or a 6-12 membered saturated or partially unsaturated fused or bridged bicyclic heterocyclic ring having 1-3 heteroatoms independently selected from nitrogen, oxygen, and sulfur, wherein: a substitutable carbon atom of Cy is optionally substituted with halogen, —(CH₂)₀₋₄R^{∘}, or —(CH₂)₀₋₄OR^{∘}, and a substitutable nitrogen atom of Cy is optionally substituted with —R^{†}; wherein R^{∘} is hydrogen or C₁₋₆ aliphatic optionally substituted by halogen or —(CH₂)₀₋₂OR^{●}, wherein R^{●} is C₁₋₄ aliphatic, and R^{†} is C₁₋₆ aliphatic. In some such embodiments, two independent occurrences of R^{∘} may be optionally taken together with their intervening atom(s), form a 3-12-membered saturated, partially unsaturated, or aryl mono- or bicyclic ring having 0-4 heteroatoms independently selected from nitrogen, oxygen, or sulfur. In certain embodiments, the present invention provides a compound of formula I, wherein R³ is —(CH₂)ₙOCy where Cy is a 3-9 membered saturated or partially unsaturated monocyclic heterocyclic ring having 1-3 heteroatoms independently selected from nitrogen, oxygen, and sulfur, wherein said ring is optionally substituted with oxo, halogen; —(CH₂)₀₋₄R^{∘}; —(CH₂)₀₋₄OR^{∘}. In some such embodiments, R^{∘} is as defined above and described herein. In certain embodiments, the present invention provides a compound of formula I, wherein R³ is —(CH₂)ₙOCy where Cy is a 3-9 membered saturated or partially unsaturated monocyclic heterocyclic ring having 1-3 heteroatoms independently selected from nitrogen, oxygen, and sulfur, wherein: a substitutable carbon atom of Cy is optionally substituted with halogen, —(CH₂)₀₋₄R^{∘}, or —(CH₂)₀₋₄OR^{∘}, and a substitutable nitrogen atom of Cy is optionally substituted with —R^{†}, wherein R^{∘} is hydrogen or C₁₋₆ aliphatic optionally substituted by halogen or —(CH₂)₀₋₂OR^{●}, wherein R^{●} is C₁₋₄ aliphatic, and R^{†} is C₁₋₆ aliphatic. In some such embodiments, two independent occurrences of R^{∘} may be optionally taken together with their intervening atom(s), form a 3-12-membered saturated, partially unsaturated, or aryl mono- or bicyclic ring having 0-4 heteroatoms independently selected from nitrogen, oxygen, or sulfur. In certain embodiments, the present invention provides a compound of formula I, wherein R³ is —(CH₂)ₙOCy where Cy is a 6-12 membered saturated or partially unsaturated fused or bridged bicyclic heterocyclic ring having 1-3 heteroatoms independently selected from nitrogen, oxygen, and sulfur, wherein said ring is optionally substituted with oxo, halogen; —(CH₂)₀₋₄R^{∘}; —(CH₂)₀₋₄OR^{∘}. In some such embodiments, R^{∘} is as defined above and described herein. In certain embodiments, the present invention provides a compound of formula I wherein R³ is —(CH₂)ₙOCy where Cy is a 6-12 membered saturated or partially unsaturated fused or bridged bicyclic heterocyclic ring having 1-3 heteroatoms independently selected from nitrogen, oxygen, and sulfur, wherein: a substitutable carbon atom of Cy is optionally substituted with halogen, —(CH₂)₀₋₄R^{∘}, or —(CH₂)₀₋₄OR^{∘}, and a substitutable nitrogen atom of Cy is optionally substituted with —R^{†}, wherein R^{∘} is hydrogen or C₁₋₆ aliphatic optionally substituted by halogen or —(CH₂)₀₋₂OR^{●}, wherein R^{●} is C₁₋₄ aliphatic, and R^{†} is C₁₋₆ aliphatic. In some such embodiments, two independent occurrences of R^{∘} may be optionally taken together with their intervening atom(s), form a 3-12-membered saturated, partially unsaturated, or aryl mono- or bicyclic ring having 0-4 heteroatoms independently selected from nitrogen, oxygen, or sulfur.

In certain embodiments, the present invention provides a compound of formula I or III, wherein R³ is C₁₋₆ aliphatic substituted by —(CH₂)₀₋₄OR^{∘}, wherein R^{∘} is hydrogen or C₁₋₆ aliphatic, wherein each R^{∘} may be substituted by halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●}. In some such embodiments, R^{●} is C₁₋₄ aliphatic. In certain embodiments, the present invention provides a compound of formula I or III, wherein R³ is C₁₋₆ aliphatic substituted by —(CH₂)₀₋₄OR^{∘}, wherein R^{∘} is hydrogen. In certain embodiments, the present invention provides a compound of formula I or III, wherein R³ is C₁₋₆ aliphatic substituted by —(CH₂)₀₋₄OR^{∘}, wherein R^{∘} is C₁₋₆ aliphatic, wherein each R^{∘} may be substituted by halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●}. In some such embodiments, R^{●} is C₁₋₄ aliphatic.

In certain embodiments, the present invention provides a compound of formula I, wherein R³ is —(CH₂)ₘOR, wherein R is hydrogen or optionally substituted C₁₋₆ aliphatic. In certain embodiments, the present invention provides a compound of formula I, wherein R³ is —(CH₂)ₘOR, wherein R is hydrogen. In certain embodiments, the present invention provides a compound of formula I, wherein R³ is —(CH₂)ₘOR, wherein R is optionally substituted C₁₋₆ aliphatic. In certain embodiments, the present invention provides a compound of formula I, wherein R³ is —(CH₂)ₘOR, wherein R is C₁₋₆ aliphatic substituted with oxo, halogen, —CN, —(CH₂)₀₋₄R^{∘}, —(CH₂)₀₋₄OR^{∘}, or —(CH₂)₀₋₄S(O)₂R^{∘}. In some such embodiments, R^{∘} is as defined above and described herein. In certain embodiments, the present invention provides a compound of formula I, wherein R³ is —(CH₂)ₘOR, wherein R is C₁₋₆ alkyl substituted with oxo, halogen, —CN, —(CH₂)₀₋₄R^{∘}, —(CH₂)₀₋₄OR^{∘}, or —(CH₂)₀₋₄S(O)₂R^{∘}. In some such embodiments, R^{∘}is as defined above and described herein. In certain embodiments, the present invention provides a compound of formula I, wherein R³ is —(CH₂)ₘOR, wherein R is C₁₋₆ alkyl substituted with oxo, halogen, —CN, —(CH₂)₀₋₄R^{∘}, —(CH₂)₀₋₄OR^{∘}, —(CH₂)₀₋₄N(R^{∘})₂, or —(CH₂)₀₋₄S(O)₂R^{∘}, wherein R^{∘} is C₁₋₆ aliphatic or a 5-6-membered saturated, partially unsaturated, or aryl ring having 0-4 heteroatoms independently selected from nitrogen, oxygen, or sulfur, wherein R^{∘} is optionally substituted with —(CH₂)₀₋₂R^{●}, wherein R^{●} is C₁₋₄ aliphatic. In certain embodiments, the present invention provides a compound of formula I, wherein R³ is —(CH₂)ₘOR, wherein R is ethyl substituted with oxo, halogen, —CN, —(CH₂)₀₋₄R^{∘}, —(CH₂)₀₋₄OR^{∘}, or —(CH₂)₀₋₄S(O)₂R^{∘}. In some such embodiments, R^{∘} is as defined above and described herein. In certain embodiments, the present invention provides a compound of formula I, wherein R³ is —(CH₂)ₘOR, wherein R is C₁₋₆ aliphatic substituted with oxo, halogen, —CN, —(CH₂)₀₋₄R^{∘}, —(CH₂)₀₋₄OR^{∘}, or —(CH₂)₀₋₄S(O)₂R^{∘}, wherein R^{∘} is independently hydrogen or C₁-6 aliphatic, wherein each R^{∘} may be substituted by halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●}. In some such embodiments, R^{●} is as defined above and described herein. In certain embodiments, the present invention provides a compound of formula I, wherein R³ is —(CH₂)ₘOR, wherein R is ethyl substituted with oxo, halogen, —CN, —(CH₂)₀₋₄R^{∘}, —(CH₂)₀₋₄OR^{∘}, —(CH₂)₀₋₄N(R^{∘})₂, or —(CH₂)₀₋₄S(O)₂R^{∘}, wherein R^{∘} is C₁₋₆ aliphatic or a 5-6-membered saturated, partially unsaturated, or aryl ring having 0-4 heteroatoms independently selected from nitrogen, oxygen, or sulfur, wherein R^{∘} is optionally substituted with —(CH₂)₀₋₂R^{●}, wherein R^{●} is C₁₋₄ aliphatic.

In certain embodiments, the present invention provides a compound of formula I or III, wherein R³ is C₁₋₆ aliphatic substituted by —(CH₂)₀₋₄N(R^{∘})₂, wherein each R^{∘} is independently hydrogen or C₁₋₆ aliphatic, wherein each R^{∘} may be substituted by halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●}. In some such embodiments, R^{●} is C₁₋₄ aliphatic. In certain embodiments, the present invention provides a compound of formula I or III, wherein R³ is C₁₋₆ aliphatic substituted by —(CH₂)₀₋₄N(R^{∘})₂, wherein each R^{∘} is hydrogen. In certain embodiments, the present invention provides a compound of formula I or III, wherein R³ is C₁₋₆ aliphatic substituted by —(CH₂)₀₋₄N(R^{∘})₂, wherein each R^{∘} is C₁₋₆ aliphatic, wherein each R^{∘} may be substituted by halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●}. In some such embodiments, R^{∘} is C₁₋₄ aliphatic.

In certain embodiments, the present invention provides a compound of formula I, wherein R³ is —(CH₂)ₘN(R)₂, wherein each R is independently hydrogen or optionally substituted C₁₋₆ aliphatic. In certain embodiments, the present invention provides a compound of formula I, wherein R³ is —(CH₂)ₘN(R)₂, wherein each R is hydrogen. In certain embodiments, the present invention provides a compound of formula I, wherein R³ is —(CH₂)ₘN(R)₂, wherein each R is optionally substituted C₁₋₆ aliphatic. In certain embodiments, the present invention provides a compound of formula I, wherein R³ is —(CH₂)ₘN(R)₂, wherein R is C₁₋₆ aliphatic substituted with oxo, halogen, —CN, —(CH₂)₀₋₄R^{∘}, —(CH₂)₀₋₄OR^{∘}, or —(CH₂)₀₋₄S(O)₂R^{∘}. In some such embodiments, R^{∘} is as defined above and described herein. In certain embodiments, the present invention provides a compound of formula I, wherein R³ is —(CH₂)ₘN(R)₂, wherein each R is C₁₋₆ alkyl substituted with oxo, halogen, —CN, —(CH₂)₀₋₄R^{∘}, —(CH₂)₀₋₄OR^{∘}, or —(CH₂)₀₋₄S(O)₂R^{∘}. In some such embodiments, R^{∘} is as defined above and described herein. In certain embodiments, the present invention provides a compound of formula I, wherein R³ is —(CH₂)ₘN(R)₂, wherein each R is C₁₋₆ alkyl optionally substituted with oxo, halogen, —CN, —(CH₂)₀₋₄R^{∘}, —(CH₂)₀₋₄OR^{∘}, or —(CH₂)₀₋₄S(O)₂R^{∘}, wherein R^{∘} is C₁₋₆ aliphatic. In certain embodiments, the present invention provides a compound of formula I, wherein R³ is —(CH₂)ₘN(R)₂, wherein each R is ethyl substituted with oxo, halogen, —CN, —(CH₂)₀₋₄R^{∘}, —(CH₂)₀₋₄OR^{∘}, or —(CH₂)₀₋₄S(O)₂R^{∘}. In some such embodiments, R^{∘} is as defined above and described herein. In certain embodiments, the present invention provides a compound of formula I, wherein R³ is —(CH₂)ₘN(R)₂, wherein each R is ethyl substituted with oxo, halogen, —CN, —(CH₂)₀₋₄R^{∘}, —(CH₂)₀₋₄OR^{∘}, or —(CH₂)₀₋₄S(O)₂R^{∘}, wherein R^{∘} is C₁₋₆ aliphatic. In certain embodiments, the present invention provides a compound of formula I, wherein R³ is —(CH₂)ₘN(R)₂, wherein R is C₁₋₆ aliphatic substituted with oxo, halogen, —CN, —(CH₂)₀₋₄R^{∘}, —(CH₂)₀₋₄OR^{∘}, or —(CH₂)₀₋₄S(O)₂R^{∘}, wherein R^{∘} is C₁₋₆ aliphatic, wherein each R^{∘} may be substituted by halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●}. In some such embodiments, R^{∘} is C₁₋₄ aliphatic.

In certain embodiments, the present invention provides a compound of formula I or III, wherein R³ is —CH₂N(R^{∘})₂, wherein each R^{∘} is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur; wherein R^{∘} may be substituted by halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●}. In some such embodiments, R^{●} is C₁₋₄ aliphatic.

In certain embodiments, the present invention provides a compound of formula I, wherein R³ is —CH₂N(R)₂. In certain embodiments, the present invention provides a compound of formula I, wherein R³ is —CH₂N(R)₂, wherein each R is independently hydrogen or optionally substituted C₁₋₆ aliphatic. In certain embodiments, the present invention provides a compound of formula I, wherein R³ is —CH₂N(R)₂, wherein each R is independently hydrogen or C₁₋₆ aliphatic substituted with oxo, halogen, —CN, —(CH₂)₀₋₄R^{∘}, —(CH₂)₀₋₄OR^{∘}, or —(CH₂)₀₋₄S(O)₂R^{∘}. In some such embodiments, R^{∘} is as defined above and described herein. In certain embodiments, the present invention provides a compound of formula I, wherein R³ is —CH₂N(R)₂, wherein each R is independently hydrogen or C₁₋₆ aliphatic optionally substituted with oxo, halogen, —CN, —(CH₂)₀₋₄R^{∘}, —(CH₂)₀₋₄OR^{∘}, or —(CH₂)₀₋₄S(O)₂R^{∘}, wherein R^{∘} is C₁₋₆ aliphatic. In certain embodiments, the present invention provides a compound of formula I, wherein R³ is —CH₂N(R)₂, wherein each R is independently hydrogen or C₁₋₆ aliphatic substituted with oxo, halogen, —CN, —(CH₂)₀₋₄R^{∘}, —(CH₂)₀₋₄OR^{∘}, or —(CH₂)₀₋₄S(O)₂R^{∘}. In some such embodiments, R^{∘} is as defined above and described herein. In certain embodiments, the present invention provides a compound of formula I, wherein R³ is —CH₂N(R)₂, wherein each R is independently hydrogen or C₁₋₆ aliphatic optionally substituted with oxo, halogen, —CN, —(CH₂)₀₋₄R^{∘}, —(CH₂)₀₋₄OR^{∘}, or —(CH₂)₀₋₄S(O)₂R^{∘}, wherein R^{∘} is C₁₋₆ aliphatic, or two R groups on the same nitrogen are taken together with the nitrogen to form a 3-7 membered saturated or partially unsaturated heterocyclic ring having 1-3 heteroatoms selected from nitrogen, oxygen, or sulfur.

In certain embodiments, the present invention provides a compound of formula I or III, wherein R³ is C₁₋₆ aliphatic substituted by —CH₂OR^{∘}, wherein R^{∘} is hydrogen or C₁₋₆ aliphatic, wherein each R^{∘} may be substituted by halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●}. In some such embodiments, R^{●} is C₁₋₄ aliphatic. In certain embodiments, the present invention provides a compound of formula I or III, wherein R³ is C₁₋₆ aliphatic substituted by —CH₂OR^{∘}, wherein R^{∘} is hydrogen. In certain embodiments, the present invention provides a compound of formula I or III, wherein R³ is C₁₋₆ aliphatic substituted by —CH₂OR^{∘}, wherein R^{∘} is C₁₋₆ aliphatic, wherein each R^{∘} may be substituted by halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●}. In some such embodiments, R^{●} is C₁₋₄ aliphatic.

In certain embodiments, the present invention provides a compound of formula I, wherein R³ is —CH₂OR, wherein R is hydrogen or optionally substituted C₁₋₆ aliphatic. In certain embodiments, the present invention provides a compound of formula I, wherein R³ is —CH₂OR, wherein R is hydrogen. In certain embodiments, the present invention provides a compound of formula I, wherein R³ is —CH₂OR, wherein R is optionally substituted C₁₋₆ aliphatic. In certain embodiments, the present invention provides a compound of formula I, wherein R³ is —CH₂OR, wherein R is C₁₋₆ aliphatic substituted with oxo, halogen, —CN, —(CH₂)₀₋₄R^{∘}, —(CH₂)₀₋₄OR^{∘}, or —(CH₂)₀₋₄S(O)₂R^{∘}. In some such embodiments, R^{∘} is as defined above and described herein. In certain embodiments, the present invention provides a compound of formula I, wherein R³ is —CH₂OR, wherein R is C₁₋₆ aliphatic substituted with oxo, halogen, —CN, —(CH₂)₀₋₄R^{∘}, —(CH₂)₀₋₄OR^{∘}, or —(CH₂)₀₋₄S(O)₂R^{∘}, wherein R^{∘} is C₁₋₆ aliphatic. In certain embodiments, the present invention provides a compound of formula I, wherein R³ is —CH₂OR, wherein R is C₁₋₆ alkyl substituted with oxo, halogen, —CN, —(CH₂)₀₋₄R^{∘}, —(CH₂)₀₋₄OR^{∘}, or —(CH₂)₀₋₄S(O)₂R^{∘}. In some embodiments, R^{∘} is as defined above and described herein. In certain embodiments, the present invention provides a compound of formula I, wherein R³ is —CH₂OR, wherein R is C₁₋₆ alkyl substituted with oxo, halogen, —CN, —(CH₂)₀₋₄R^{∘}, —(CH₂)₀₋₄OR^{∘}, or —(CH₂)₀₋₄S(O)₂R^{∘}, wherein R^{∘} is C₁₋₆ aliphatic. In certain embodiments, the present invention provides a compound of formula I, wherein R³ is —CH₂OR, wherein R is ethyl substituted with oxo, halogen, —CN, —(CH₂)₀₋₄R^{∘}, —(CH₂)₀₋₄OR^{∘}, or —(CH₂)₀₋₄S(O)₂R^{∘}. In some such embodiments, R^{∘} is as defined above and described herein. In some embodiments of formula I, R³ is —CH₂OR, wherein R is ethyl substituted with oxo, halogen, —CN, —(CH₂)₀₋₄R^{∘}, —(CH₂)₀₋₄OR^{∘}, or —(CH₂)₀₋₄S(O)₂R^{∘}, R^{∘} is C₁₋₆ aliphatic. In certain embodiments, the present invention provides a compound of formula I, wherein R³ is —CH₂OR, wherein R is C₁₋₆ aliphatic substituted with oxo, halogen, —CN, —(CH₂)₀₋₄R^{∘}, —(CH₂)₀₋₄OR^{∘}, or —(CH₂)₀₋₄S(O)₂R^{∘}, wherein each R^{∘} is independently hydrogen or C₁₋₆ aliphatic, wherein R^{∘} may be substituted by halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●}. In some such embodiments, R^{●} is C₁₋₄ aliphatic.

In certain embodiments, the present invention provides a compound of formula I or III, wherein R³ is —(CH₂)ₘCy where Cy is optionally substituted piperidinyl. In certain embodiments, the present invention provides a compound of formula I or III, wherein R³ is —(CH₂)ₘCy where Cy is piperidinyl optionally substituted with oxo, halogen, —(CH₂)₀₋₄R^{∘}, or —(CH₂)₀₋₄OR^{∘}, wherein R^{∘} is as defined above and described herein. In certain embodiments, the present invention provides a compound of formula I or III, wherein R³ is —(CH₂)ₘCy where Cy is piperidinyl optionally substituted with oxo, halogen, —(CH₂)₀₋₄R^{∘}, or —(CH₂)₀₋₄OR^{∘}, wherein each R^{∘} is independently C₁₋₆ aliphatic, wherein two independent occurrences of R^{∘} may be optionally taken together with their intervening atom(s), form a 3-12-membered saturated, partially unsaturated, or aryl mono- or bicyclic ring having 0-4 heteroatoms independently selected from nitrogen, oxygen, or sulfur. In certain embodiments, the present invention provides a compound of formula I or III, wherein R³ is —(CH₂)ₙOCy where Cy is optionally substituted oxetanyl. In certain embodiments, the present invention provides a compound of formula I or III, wherein R³ is —(CH₂)ₙOCy where Cy is oxetanyl optionally substituted with oxo, halogen, —(CH₂)₀₋₄R^{∘}, or —(CH₂)₀₋₄OR^{∘}, wherein R^{∘} is C₁₋₆ aliphatic. In certain embodiments, the present invention provides a compound of formula I or III, wherein R³ is —(CH₂)ₙOCy where Cy is oxetanyl optionally substituted with oxo, halogen, —(CH₂)₀₋₄R^{∘}, or —(CH₂)₀₋₄OR^{∘}, wherein R^{∘} is C₁₋₆ aliphatic, wherein two independent occurrences of R^{∘} may be optionally taken together with their intervening atom(s), form a 3-12-membered saturated, partially unsaturated, or aryl mono- or bicyclic ring having 0-4 heteroatoms independently selected from nitrogen, oxygen, or sulfur. In certain embodiments, the present invention provides a compound of formula I or III, wherein R³ is —(CH₂)ₙOCy where Cy is oxetanyl optionally substituted with oxo, halogen, —(CH₂)₀₋₄R^{∘}, or —(CH₂)₀₋₄OR^{∘}, wherein each R^{∘} is C₁₋₆ aliphatic, wherein two independent occurrences of R^{∘} may be optionally taken together with their intervening atom(s), form a 3-12-membered saturated, partially unsaturated, or aryl mono- or bicyclic ring having 0-4 heteroatoms independently selected from nitrogen, oxygen, or sulfur.

In certain embodiments, the present invention provides a compound of formula I, wherein R³ is —(CH₂)ₘCy where Cy is optionally substituted piperidinyl. In certain embodiments, the present invention provides a compound of formula I, wherein R³ is —(CH₂)ₘCy where Cy is piperidinyl optionally substituted with oxo, halogen, —(CH₂)₀₋₄R^{∘}, or —(CH₂)₀₋₄OR^{∘}, wherein each R^{∘} is C₁₋₆ aliphatic, wherein two independent occurrences of R^{∘} may be optionally taken together with their intervening atom(s), form a 3-12-membered saturated, partially unsaturated, or aryl mono- or bicyclic ring having 0-4 heteroatoms independently selected from nitrogen, oxygen, or sulfur. In certain embodiments, the present invention provides a compound of formula I, wherein R³ is —(CH₂)ₙOCy where Cy is optionally substituted oxetanyl. In certain embodiments, the present invention provides a compound of formula I, wherein R³ is —(CH₂)ₙOCy where Cy is oxetanyl optionally substituted with oxo, halogen, —(CH₂)₀₋₄R^{∘}, or —(CH₂)₀₋₄OR^{∘}, wherein each R^{∘} is C₁₋₆ aliphatic, wherein two independent occurrences of R^{∘} may be optionally taken together with their intervening atom(s), form a 3-12-membered saturated, partially unsaturated, or aryl mono- or bicyclic ring having 0-4 heteroatoms independently selected from nitrogen, oxygen, or sulfur. In certain embodiments, the present invention provides a compound of formula I, wherein R³ is —(CH₂)ₙOCy where Cy is oxetanyl optionally substituted with oxo, halogen, —(CH₂)₀₋₄R^{∘}, or —(CH₂)₀₋₄OR^{∘}, wherein each R^{∘} is C₁₋₆ aliphatic, wherein two independent occurrences of R^{∘} may be optionally taken together with their intervening atom(s), form a 3-12-membered saturated, partially unsaturated, or aryl mono- or bicyclic ring having 0-4 heteroatoms independently selected from nitrogen, oxygen, or sulfur.

In certain embodiments, the present disclosure provides a compound of formula I selected from those depicted in Table 1, below.

TABLE 1

| Compound # | Structure |
|---|---|
| I-1 | 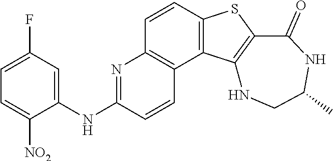 [SMILES: C[C@@H]1CNc2c(sc3ccc4nc(Nc5cc(F)ccc5[N+](=O)[O-])ccc4c23)C(=O)N1] |
| I-2 | 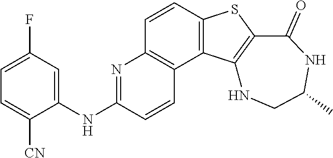 [SMILES: C[C@@H]1CNc2c(sc3ccc4nc(Nc5cc(F)ccc5C#N)ccc4c23)C(=O)N1] |
| I-3 | 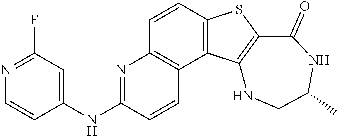 [SMILES: C[C@@H]1CNc2c(sc3ccc4nc(Nc5ccnc(F)c5)ccc4c23)C(=O)N1] |
| I-4 | 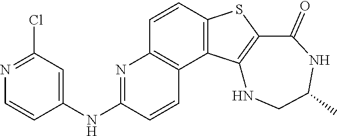 [SMILES: C[C@@H]1CNc2c(sc3ccc4nc(Nc5ccnc(Cl)c5)ccc4c23)C(=O)N1] |
| I-5 | 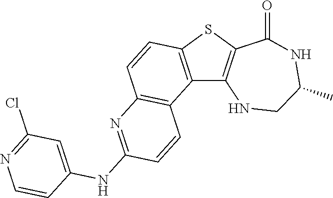 [SMILES: C[C@@H]1CNc2c(sc3ccc4nc(Nc5ccnc(Cl)c5)ccc4c23)C(=O)N1] |
| I-6 | 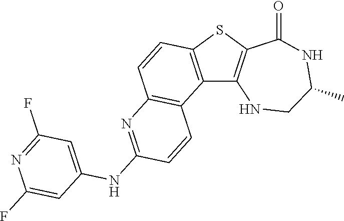 [SMILES: C[C@@H]1CNc2c(sc3ccc4nc(Nc5cc(F)nc(F)c5)ccc4c23)C(=O)N1] |
| I-7 | 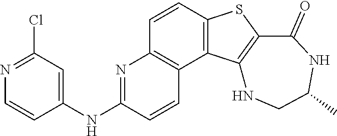 [SMILES: C[C@@H]1CNc2c(sc3ccc4nc(Nc5ccnc(Cl)c5)ccc4c23)C(=O)N1] |
| I-8 | 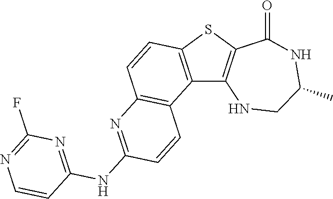 [SMILES: C[C@@H]1CNc2c(sc3ccc4nc(Nc5ccnc(F)n5)ccc4c23)C(=O)N1] |
| I-9 | 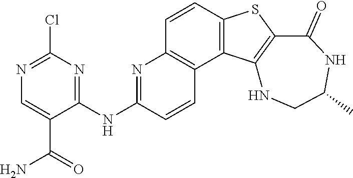 [SMILES: C[C@@H]1CNc2c(sc3ccc4nc(Nc5nc(Cl)ncc5C(N)=O)ccc4c23)C(=O)N1] |
| I-10 | 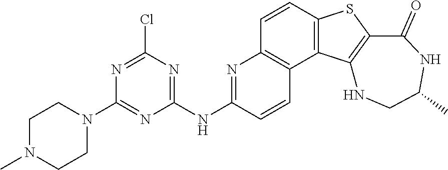 |
| I-11 | 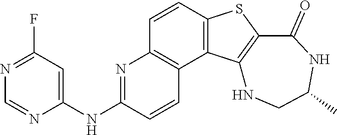 [SMILES: C[C@@H]1CNc2c(sc3ccc4nc(Nc5cc(F)ncn5)ccc4c23)C(=O)N1] |
| I-12 | 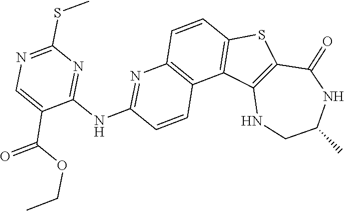 [SMILES: CCOC(=O)c1cnc(SC)nc1Nc1ccc2c(ccc3sc4c(c32)NC[C@@H](C)NC4=O)n1] |
| I-13 | 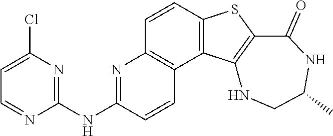 [SMILES: C[C@@H]1CNc2c(sc3ccc4nc(Nc5nccc(Cl)n5)ccc4c23)C(=O)N1] |
| I-14 | 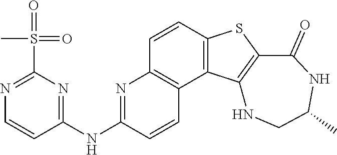 |
| I-15 | 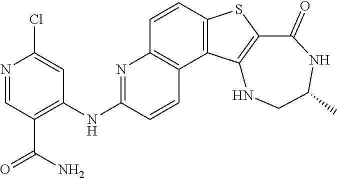 [SMILES: C[C@@H]1CNc2c(sc3ccc4nc(Nc5cc(Cl)ncc5C(N)=O)ccc4c23)C(=O)N1] |
| I-16 | 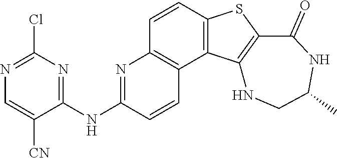 [SMILES: C[C@@H]1CNc2c(sc3ccc4nc(Nc5nc(Cl)ncc5C#N)ccc4c23)C(=O)N1] |
| I-17 | 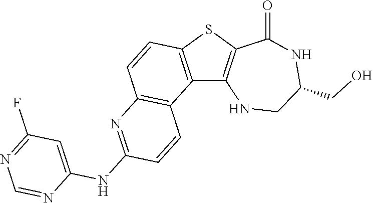 [SMILES: O=C1N[C@H](CO)CNc2c1sc1ccc3nc(Nc4cc(F)ncn4)ccc3c21] |
| I-18 | 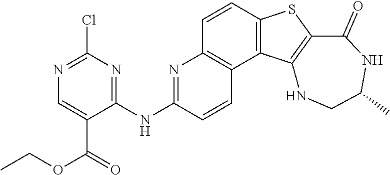 [SMILES: CCOC(=O)c1cnc(Cl)nc1Nc1ccc2c(ccc3sc4c(c32)NC[C@@H](C)NC4=O)n1] |
| I-19 | 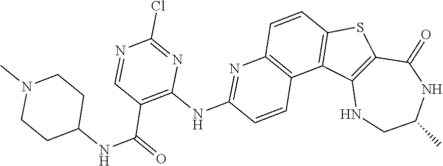 |
| I-20 | 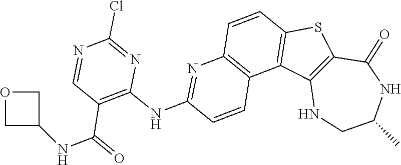 [SMILES: C[C@@H]1CNc2c(sc3ccc4nc(Nc5nc(Cl)ncc5C(=O)NC5COC5)ccc4c23)C(=O)N1] |
| I-21 | 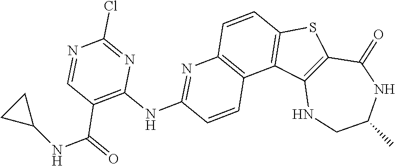 [SMILES: C[C@@H]1CNc2c(sc3ccc4nc(Nc5nc(Cl)ncc5C(=O)NC5CC5)ccc4c23)C(=O)N1] |
| I-22 | 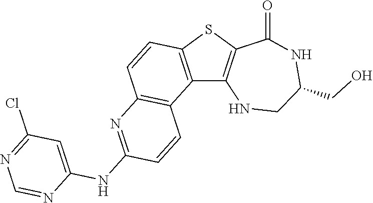 [SMILES: O=C1N[C@H](CO)CNc2c1sc1ccc3nc(Nc4cc(Cl)ncn4)ccc3c21] |
| I-23 | 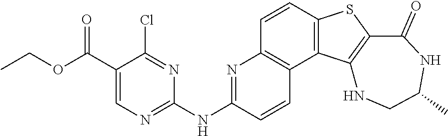 [SMILES: CCOC(=O)c1cnc(Nc2ccc3c(ccc4sc5c(c43)NC[C@@H](C)NC5=O)n2)nc1Cl] |
| I-24 | 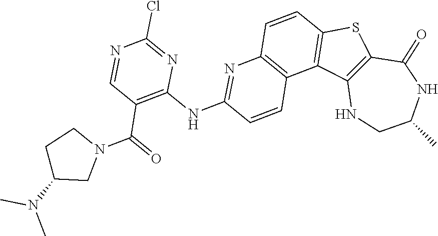 |
| I-25 | 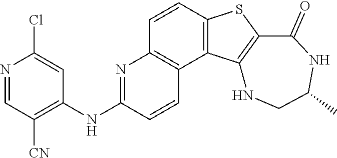 [SMILES: C[C@@H]1CNc2c(sc3ccc4nc(Nc5cc(Cl)ncc5C#N)ccc4c23)C(=O)N1] |
| I-26 | 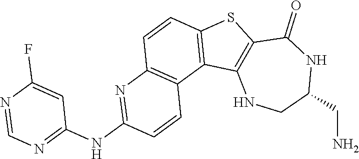 [SMILES: NC[C@@H]1CNc2c(sc3ccc4nc(Nc5cc(F)ncn5)ccc4c23)C(=O)N1] |
| I-27 | 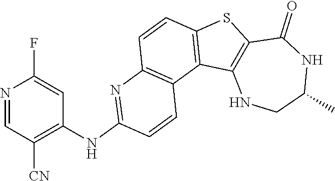 [SMILES: C[C@@H]1CNc2c(sc3ccc4nc(Nc5cc(F)ncc5C#N)ccc4c23)C(=O)N1] |
| I-28 | 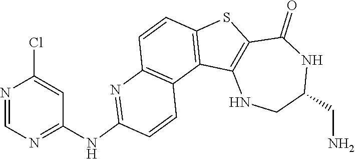 [SMILES: NC[C@@H]1CNc2c(sc3ccc4nc(Nc5cc(Cl)ncn5)ccc4c23)C(=O)N1] |
| I-29 | 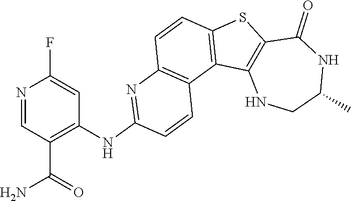 [SMILES: C[C@@H]1CNc2c(sc3ccc4nc(Nc5cc(F)ncc5C(N)=O)ccc4c23)C(=O)N1] |
| I-30 | 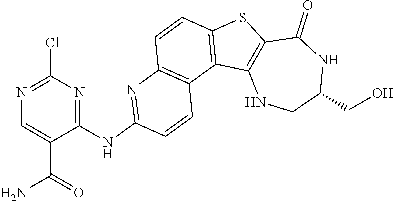 [SMILES: NC(=O)c1cnc(Cl)nc1Nc1ccc2c(ccc3sc4c(c32)NC[C@@H](CO)NC4=O)n1] |
| I-31 | 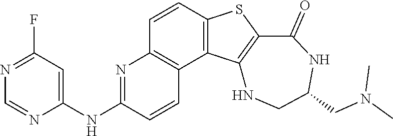 [SMILES: CN(C)C[C@@H]1CNc2c(sc3ccc4nc(Nc5cc(F)ncn5)ccc4c23)C(=O)N1] |
| I-32 | 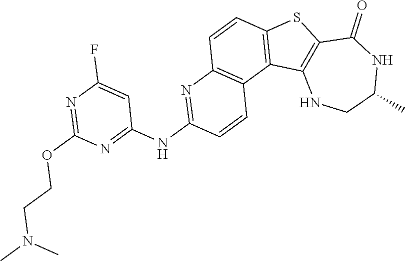 |
| I-33 | 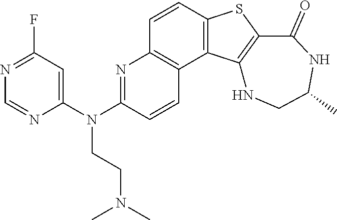 [SMILES: C[C@@H]1CNc2c(sc3ccc4nc(N(CCN(C)C)c5cc(F)ncn5)ccc4c23)C(=O)N1] |
| I-34 | 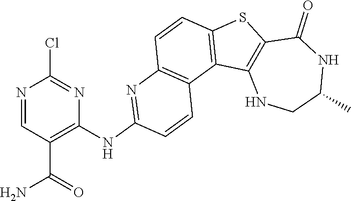 [SMILES: C[C@@H]1CNc2c(sc3ccc4nc(Nc5nc(Cl)ncc5C(N)=O)ccc4c23)C(=O)N1] |
| I-35 | 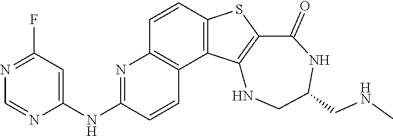 [SMILES: CNC[C@@H]1CNc2c(sc3ccc4nc(Nc5cc(F)ncn5)ccc4c23)C(=O)N1] |
| I-36 | 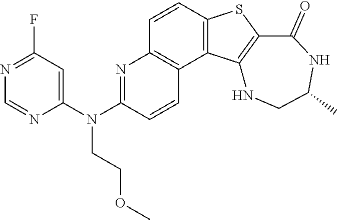 [SMILES: COCCN(c1cc(F)ncn1)c1ccc2c(ccc3sc4c(c32)NC[C@@H](C)NC4=O)n1] |
| I-37 | 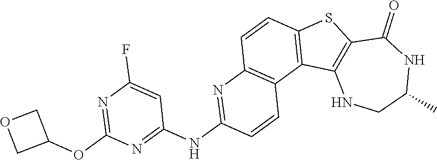 [SMILES: C[C@@H]1CNc2c(sc3ccc4nc(Nc5cc(F)nc(OC6COC6)n5)ccc4c23)C(=O)N1] |
| I-38 | 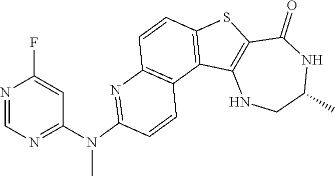 [SMILES: C[C@@H]1CNc2c(sc3ccc4nc(N(C)c5cc(F)ncn5)ccc4c23)C(=O)N1] |
| I-39 | 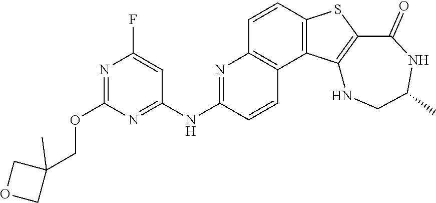 [SMILES: C[C@@H]1CNc2c(sc3ccc4nc(Nc5cc(F)nc(OCC6(C)COC6)n5)ccc4c23)C(=O)N1] |
| I-40 | 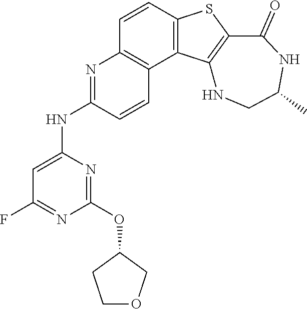 [SMILES: C[C@@H]1CNc2c(sc3ccc4nc(Nc5cc(F)nc(O[C@H]6CCOC6)n5)ccc4c23)C(=O)N1] |
| I-41 | 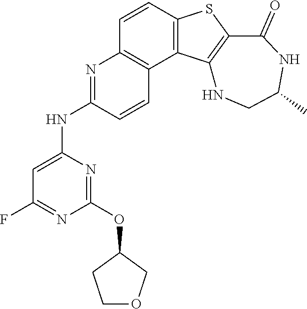 [SMILES: C[C@@H]1CNc2c(sc3ccc4nc(Nc5cc(F)nc(O[C@@H]6CCOC6)n5)ccc4c23)C(=O)N1] |
| I-42 | 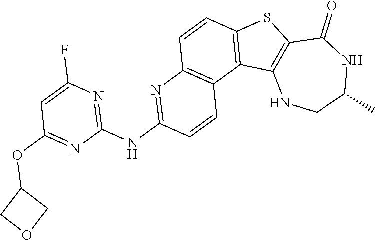 [SMILES: C[C@@H]1CNc2c(sc3ccc4nc(Nc5nc(F)cc(OC6COC6)n5)ccc4c23)C(=O)N1] |
| I-43 | 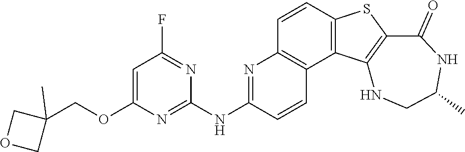 [SMILES: C[C@@H]1CNc2c(sc3ccc4nc(Nc5nc(F)cc(OCC6(C)COC6)n5)ccc4c23)C(=O)N1] |
| I-44 | 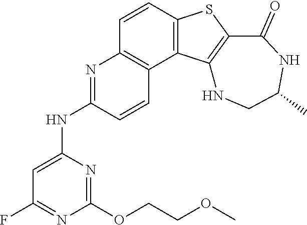 [SMILES: COCCOc1nc(F)cc(Nc2ccc3c(ccc4sc5c(c43)NC[C@@H](C)NC5=O)n2)n1] |
| I-45 | 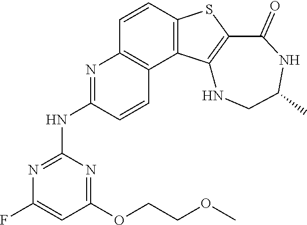 [SMILES: COCCOc1cc(F)nc(Nc2ccc3c(ccc4sc5c(c43)NC[C@@H](C)NC5=O)n2)n1] |
| I-46 | 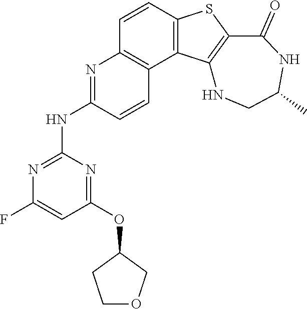 [SMILES: C[C@@H]1CNc2c(sc3ccc4nc(Nc5nc(F)cc(O[C@@H]6CCOC6)n5)ccc4c23)C(=O)N1] |
| I-47 | 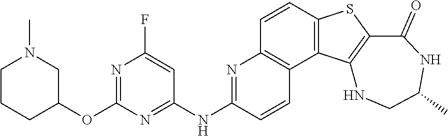 [SMILES: C[C@@H]1CNc2c(sc3ccc4nc(Nc5cc(F)nc(OC6CCCN(C)C6)n5)ccc4c23)C(=O)N1] |
| I-48 | 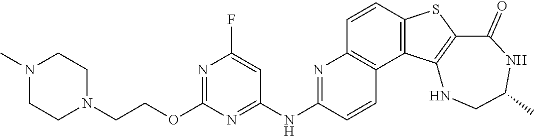 |
| I-49 | 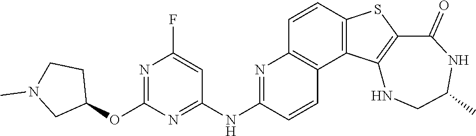 |
| I-50 | 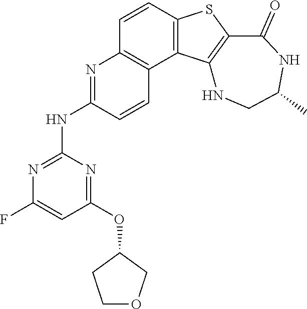 [SMILES: C[C@@H]1CNc2c(sc3ccc4nc(Nc5nc(F)cc(O[C@H]6CCOC6)n5)ccc4c23)C(=O)N1] |
| I-51 | 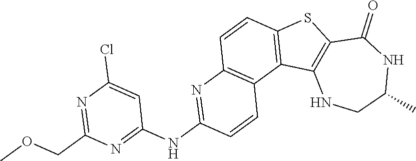 |
| I-52 | 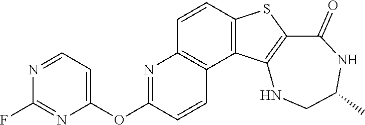 [SMILES: C[C@@H]1CNc2c(sc3ccc4nc(Oc5ccnc(F)n5)ccc4c23)C(=O)N1] |
| I-53 | 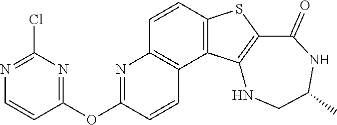 [SMILES: C[C@@H]1CNc2c(sc3ccc4nc(Oc5ccnc(Cl)n5)ccc4c23)C(=O)N1] |
| I-54 | 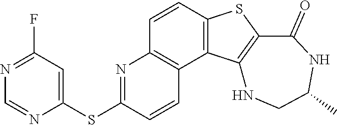 [SMILES: C[C@@H]1CNc2c(sc3ccc4nc(Sc5cc(F)ncn5)ccc4c23)C(=O)N1] |
| I-55 | 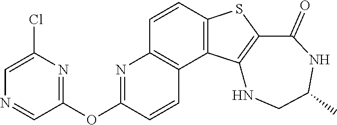 [SMILES: C[C@@H]1CNc2c(sc3ccc4nc(Oc5cncc(Cl)n5)ccc4c23)C(=O)N1] |
| I-56 | 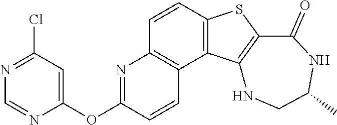 [SMILES: C[C@@H]1CNc2c(sc3ccc4nc(Oc5cc(Cl)ncn5)ccc4c23)C(=O)N1] |
| I-57 | 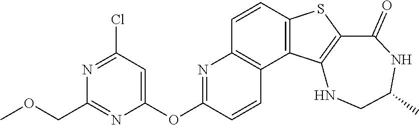 |
| I-58 | 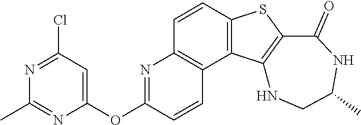 |
| I-59 | 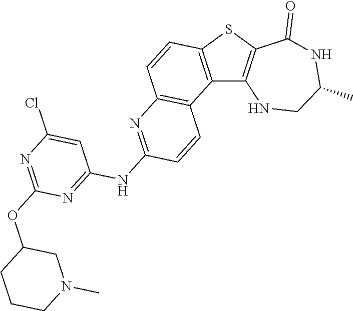 [SMILES: C[C@@H]1CNc2c(sc3ccc4nc(Nc5cc(Cl)nc(OC6CCCN(C)C6)n5)ccc4c23)C(=O)N1] |
| I-60 | 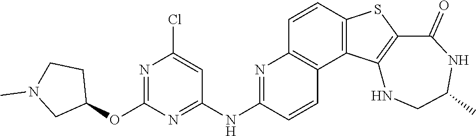 |
| I-61 | 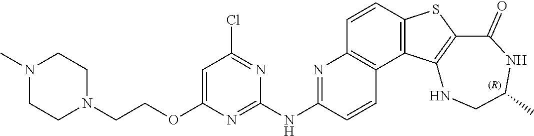 |
| I-62 | 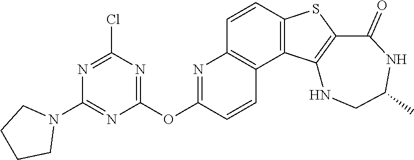 [SMILES: C[C@@H]1CNc2c(sc3ccc4nc(Oc5nc(Cl)nc(N6CCCC6)n5)ccc4c23)C(=O)N1] |
| I-63 | 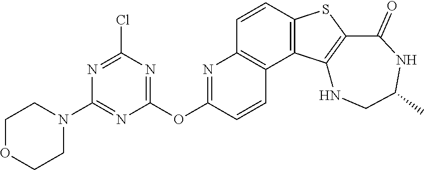 [SMILES: C[C@@H]1CNc2c(sc3ccc4nc(Oc5nc(Cl)nc(N6CCOCC6)n5)ccc4c23)C(=O)N1] |
| I-64 | 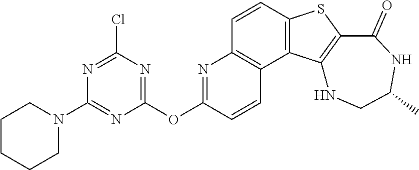 [SMILES: C[C@@H]1CNc2c(sc3ccc4nc(Oc5nc(Cl)nc(N6CCCCC6)n5)ccc4c23)C(=O)N1] |
| I-65 | 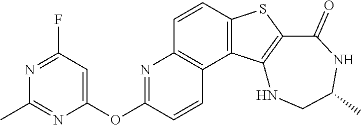 |
| I-66 | 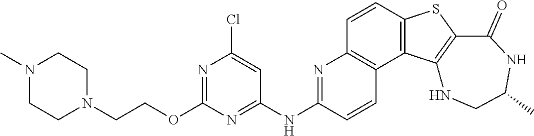 |
| I-67 | 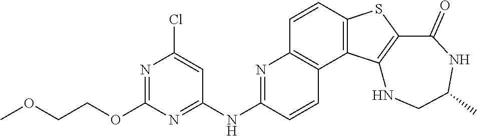 |
| I-68 | 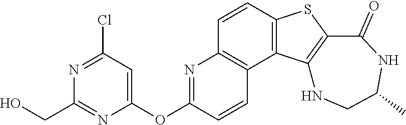 [SMILES: C[C@@H]1CNc2c(sc3ccc4nc(Oc5cc(Cl)nc(CO)n5)ccc4c23)C(=O)N1] |
| I-69 | 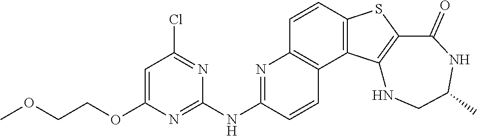 |
| I-70 | 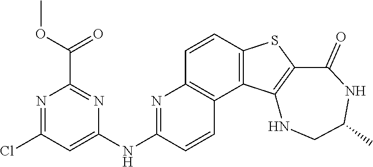 [SMILES: COC(=O)c1nc(Cl)cc(Nc2ccc3c(ccc4sc5c(c43)NC[C@@H](C)NC5=O)n2)n1] |
| I-71 | 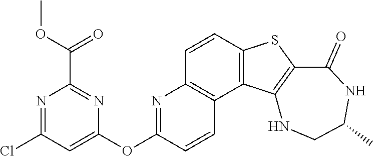 [SMILES: COC(=O)c1nc(Cl)cc(Oc2ccc3c(ccc4sc5c(c43)NC[C@@H](C)NC5=O)n2)n1] |
| I-72 | 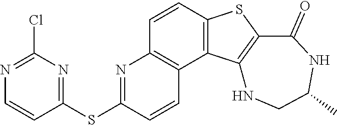 [SMILES: C[C@@H]1CNc2c(sc3ccc4nc(Sc5ccnc(Cl)n5)ccc4c23)C(=O)N1] |
| I-73 | 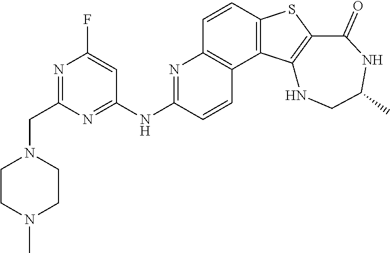 [SMILES: C[C@@H]1CNc2c(sc3ccc4nc(Nc5cc(F)nc(CN6CCN(C)CC6)n5)ccc4c23)C(=O)N1] |
| I-74 | 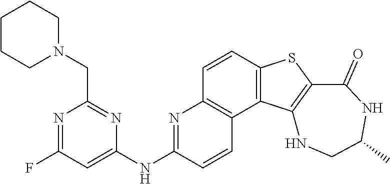 [SMILES: C[C@@H]1CNc2c(sc3ccc4nc(Nc5cc(F)nc(CN6CCCCC6)n5)ccc4c23)C(=O)N1] |
| I-75 | 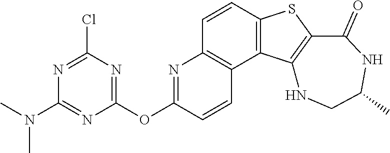 |
| I-76 | 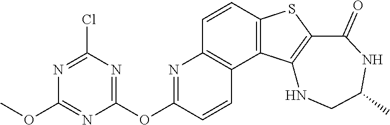 |
| I-77 | 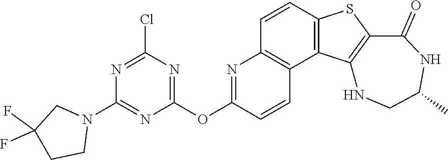 [SMILES: C[C@@H]1CNc2c(sc3ccc4nc(Oc5nc(Cl)nc(N6CCC(F)(F)C6)n5)ccc4c23)C(=O)N1] |
| I-78 | 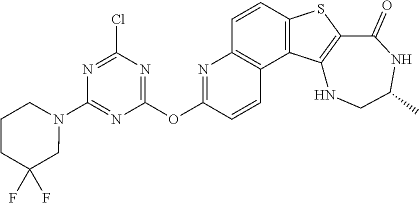 [SMILES: C[C@@H]1CNc2c(sc3ccc4nc(Oc5nc(Cl)nc(N6CCCC(F)(F)C6)n5)ccc4c23)C(=O)N1] |
| I-79 | 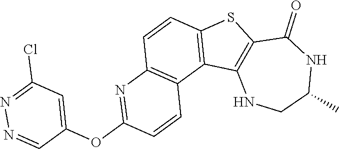 [SMILES: C[C@@H]1CNc2c(sc3ccc4nc(Oc5cnnc(Cl)c5)ccc4c23)C(=O)N1] |
| I-80 | 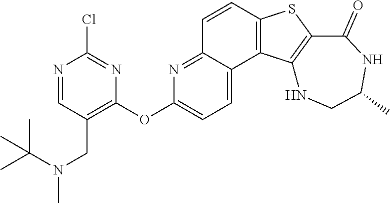 [SMILES: C[C@@H]1CNc2c(sc3ccc4nc(Oc5nc(Cl)ncc5CN(C)C(C)(C)C)ccc4c23)C(=O)N1] |
| I-81 | 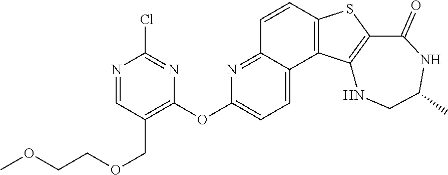 |
| I-82 | 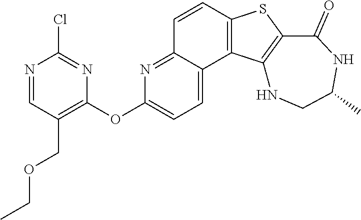 [SMILES: CCOCc1cnc(Cl)nc1Oc1ccc2c(ccc3sc4c(c32)NC[C@@H](C)NC4=O)n1] |
| I-83 | 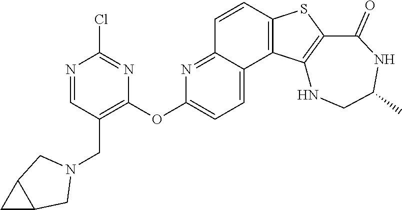 [SMILES: C[C@@H]1CNc2c(sc3ccc4nc(Oc5nc(Cl)ncc5CN5CC6CC6C5)ccc4c23)C(=O)N1] |
| I-84 | 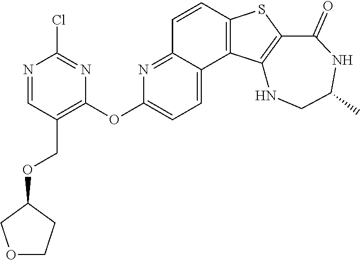 [SMILES: C[C@@H]1CNc2c(sc3ccc4nc(Oc5nc(Cl)ncc5CO[C@H]5CCOC5)ccc4c23)C(=O)N1] |
| I-85 | 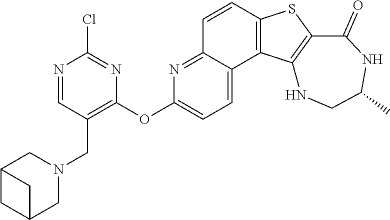 [SMILES: C[C@@H]1CNc2c(sc3ccc4nc(Oc5nc(Cl)ncc5CN5CC6CC(C6)C5)ccc4c23)C(=O)N1] |
| I-86 | 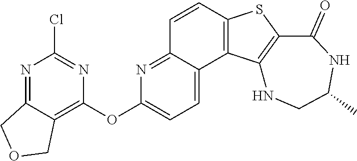 [SMILES: C[C@@H]1CNc2c(sc3ccc4nc(Oc5nc(Cl)nc6c5COC6)ccc4c23)C(=O)N1] |
| I-87 | 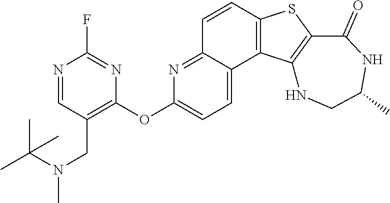 [SMILES: C[C@@H]1CNc2c(sc3ccc4nc(Oc5nc(F)ncc5CN(C)C(C)(C)C)ccc4c23)C(=O)N1] |
| I-88 | 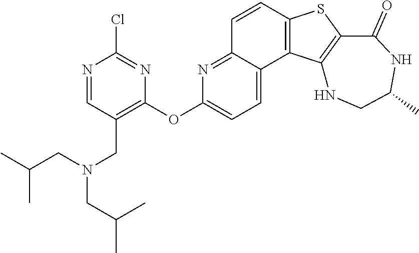 [SMILES: CC(C)CN(Cc1cnc(Cl)nc1Oc1ccc2c(ccc3sc4c(c32)NC[C@@H](C)NC4=O)n1)CC(C)C] |
| I-89 | 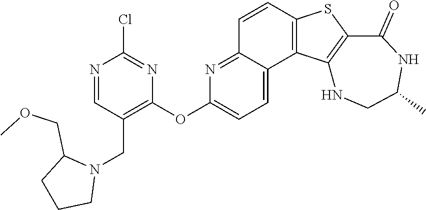 |
| I-90 | 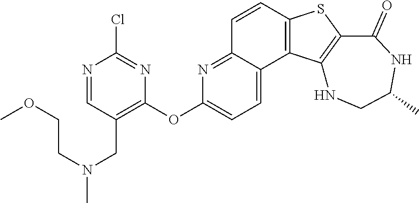 |
| I-91 | 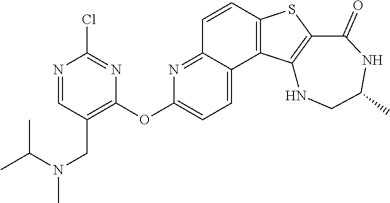 [SMILES: CC(C)N(C)Cc1cnc(Cl)nc1Oc1ccc2c(ccc3sc4c(c32)NC[C@@H](C)NC4=O)n1] |
| I-92 | 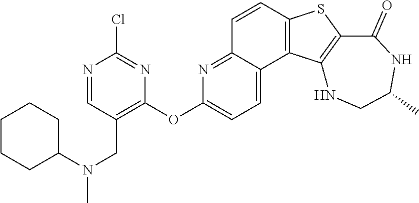 [SMILES: C[C@@H]1CNc2c(sc3ccc4nc(Oc5nc(Cl)ncc5CN(C)C5CCCCC5)ccc4c23)C(=O)N1] |
| I-93 | 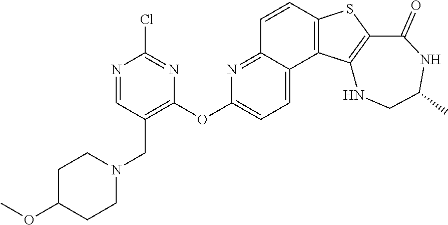 |
| I-94 | 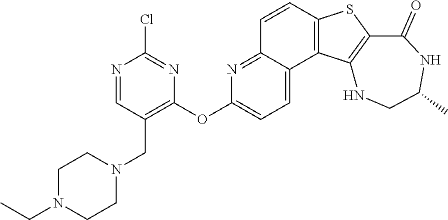 [SMILES: CCN1CCN(Cc2cnc(Cl)nc2Oc2ccc3c(ccc4sc5c(c43)NC[C@@H](C)NC5=O)n2)CC1] |
| I-95 | 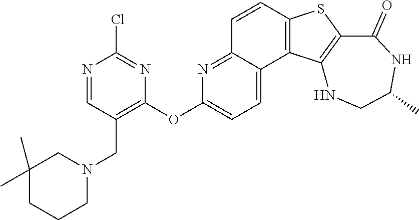 |
| I-96 | 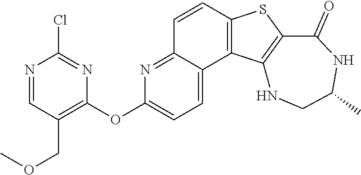 |
| I-97 | 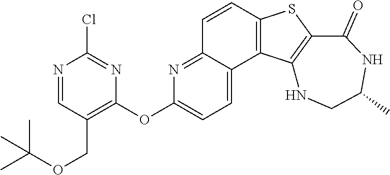 [SMILES: C[C@@H]1CNc2c(sc3ccc4nc(Oc5nc(Cl)ncc5COC(C)(C)C)ccc4c23)C(=O)N1] |
| I-98 | 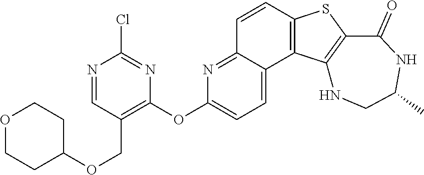 [SMILES: C[C@@H]1CNc2c(sc3ccc4nc(Oc5nc(Cl)ncc5COC5CCOCC5)ccc4c23)C(=O)N1] |
| I-99 | 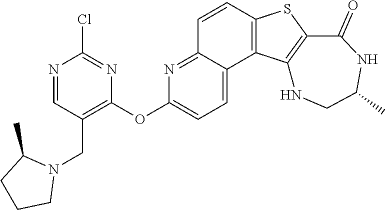 [SMILES: C[C@@H]1CNc2c(sc3ccc4nc(Oc5nc(Cl)ncc5CN5CCC[C@H]5C)ccc4c23)C(=O)N1] |
| I-100 | 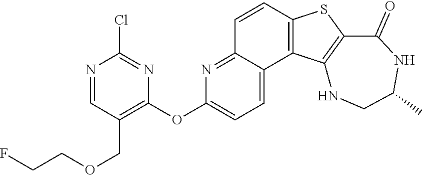 [SMILES: C[C@@H]1CNc2c(sc3ccc4nc(Oc5nc(Cl)ncc5COCCF)ccc4c23)C(=O)N1] |
| I-101 | 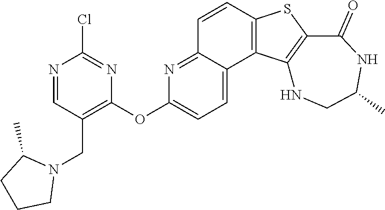 [SMILES: C[C@@H]1CNc2c(sc3ccc4nc(Oc5nc(Cl)ncc5CN5CCC[C@@H]5C)ccc4c23)C(=O)N1] |
| I-102 | 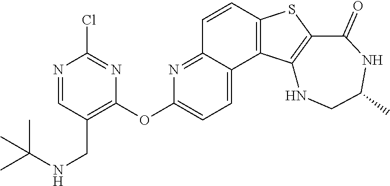 [SMILES: C[C@@H]1CNc2c(sc3ccc4nc(Oc5nc(Cl)ncc5CNC(C)(C)C)ccc4c23)C(=O)N1] |
| I-103 | 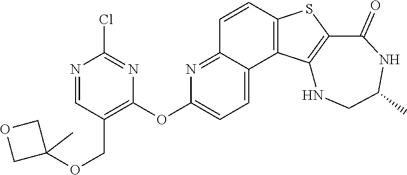 [SMILES: C[C@@H]1CNc2c(sc3ccc4nc(Oc5nc(Cl)ncc5COC5(C)COC5)ccc4c23)C(=O)N1] |
| I-104 | 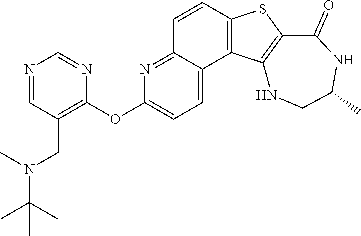 |
| I-105 | 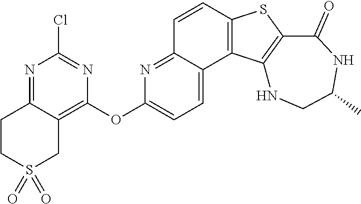 [SMILES: C[C@@H]1CNc2c(sc3ccc4nc(Oc5nc(Cl)nc6c5CS(=O)(=O)CC6)ccc4c23)C(=O)N1] |
| I-106 | 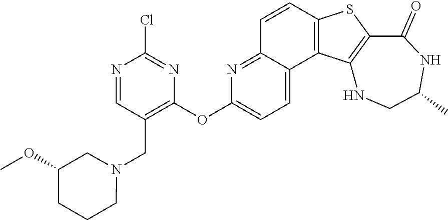 |
| I-107 | 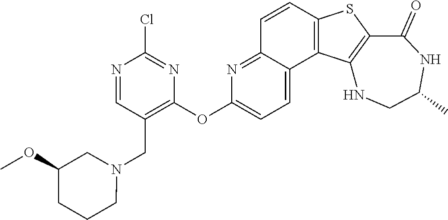 |
| I-108 | 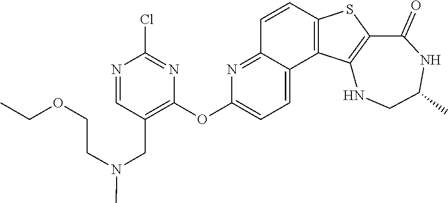 [SMILES: CCOCCN(C)Cc1cnc(Cl)nc1Oc1ccc2c(ccc3sc4c(c32)NC[C@@H](C)NC4=O)n1] |
| I-109 | 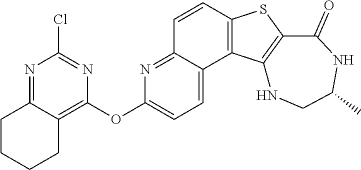 [SMILES: C[C@@H]1CNc2c(sc3ccc4nc(Oc5nc(Cl)nc6c5CCCC6)ccc4c23)C(=O)N1] |
| I-110 | 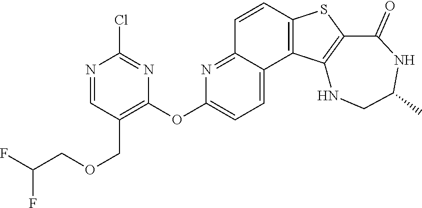 [SMILES: C[C@@H]1CNc2c(sc3ccc4nc(Oc5nc(Cl)ncc5COCC(F)F)ccc4c23)C(=O)N1] |
| I-111 | 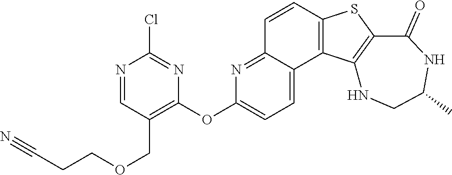 [SMILES: C[C@@H]1CNc2c(sc3ccc4nc(Oc5nc(Cl)ncc5COCCC#N)ccc4c23)C(=O)N1] |
| I-112 | 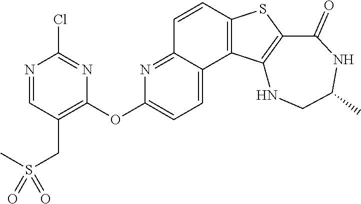 |
| I-113 | 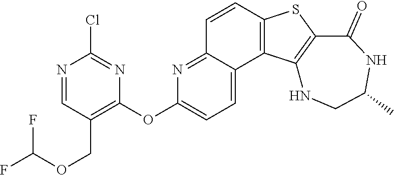 [SMILES: C[C@@H]1CNc2c(sc3ccc4nc(Oc5nc(Cl)ncc5COC(F)F)ccc4c23)C(=O)N1] |
| I-114 | 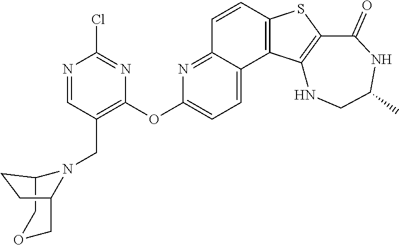 [SMILES: C[C@@H]1CNc2c(sc3ccc4nc(Oc5nc(Cl)ncc5CN5C6CCC5COC6)ccc4c23)C(=O)N1] |
| I-115 | 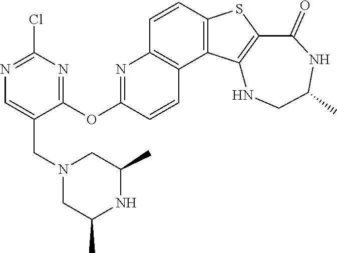 [SMILES: C[C@@H]1CNc2c(sc3ccc4nc(Oc5nc(Cl)ncc5CN5C[C@@H](C)N[C@@H](C)C5)ccc4c23)C(=O)N1] |
| I-116 | 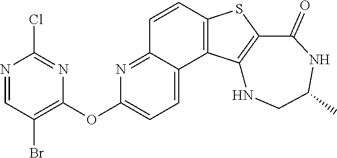 [SMILES: C[C@@H]1CNc2c(sc3ccc4nc(Oc5nc(Cl)ncc5Br)ccc4c23)C(=O)N1] 7 |
| I-117 | 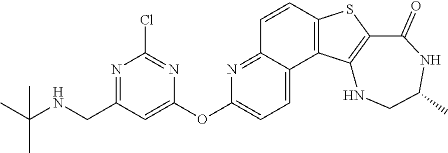 [SMILES: C[C@@H]1CNc2c(sc3ccc4nc(Oc5cc(CNC(C)(C)C)nc(Cl)n5)ccc4c23)C(=O)N1] |
| I-118 | 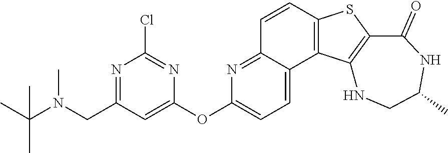 [SMILES: C[C@@H]1CNc2c(sc3ccc4nc(Oc5cc(CN(C)C(C)(C)C)nc(Cl)n5)ccc4c23)C(=O)N1] |
| I-119 | 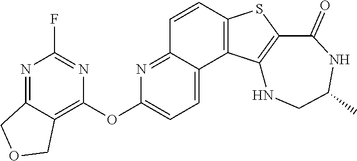 [SMILES: C[C@@H]1CNc2c(sc3ccc4nc(Oc5nc(F)nc6c5COC6)ccc4c23)C(=O)N1] |
| I-120 | 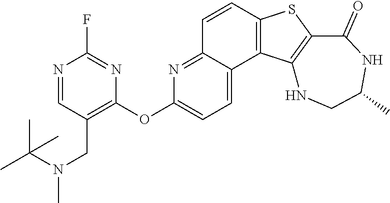 [SMILES: C[C@@H]1CNc2c(sc3ccc4nc(Oc5nc(F)ncc5CN(C)C(C)(C)C)ccc4c23)C(=O)N1] |
| I-121 | 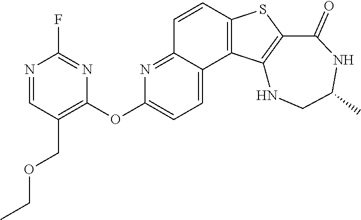 [SMILES: CCOCc1cnc(F)nc1Oc1ccc2c(ccc3sc4c(c32)NC[C@@H](C)NC4=O)n1] |
| I-122 | 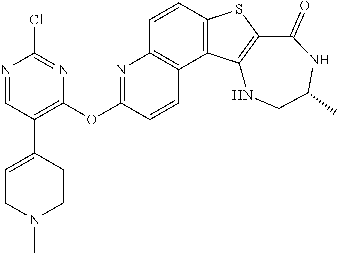 [SMILES: C[C@@H]1CNc2c(sc3ccc4nc(Oc5nc(Cl)ncc5C5=CCN(C)CC5)ccc4c23)C(=O)N1] |
| I-123 | 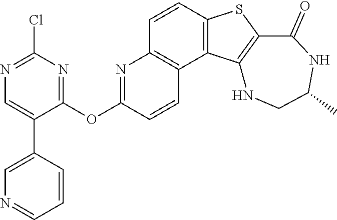 [SMILES: C[C@@H]1CNc2c(sc3ccc4nc(Oc5nc(Cl)ncc5-c5cccnc5)ccc4c23)C(=O)N1] |
| I-124 | 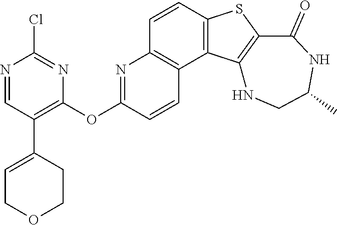 [SMILES: C[C@@H]1CNc2c(sc3ccc4nc(Oc5nc(Cl)ncc5C5=CCOCC5)ccc4c23)C(=O)N1] |
| I-125 | 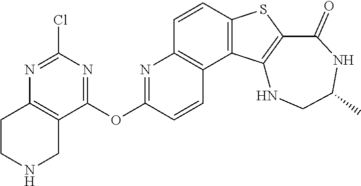 [SMILES: C[C@@H]1CNc2c(sc3ccc4nc(Oc5nc(Cl)nc6c5CNCC6)ccc4c23)C(=O)N1] |
| I-126 | 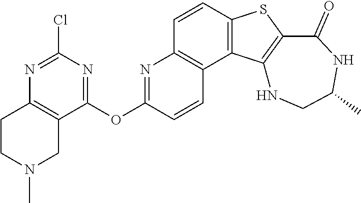 [SMILES: C[C@@H]1CNc2c(sc3ccc4nc(Oc5nc(Cl)nc6c5CN(C)CC6)ccc4c23)C(=O)N1] |
| I-127 | 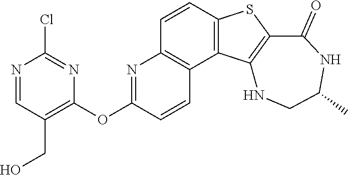 [SMILES: C[C@@H]1CNc2c(sc3ccc4nc(Oc5nc(Cl)ncc5CO)ccc4c23)C(=O)N1] |
| I-128 | 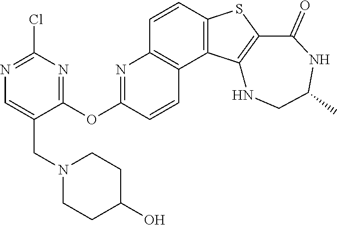 [SMILES: C[C@@H]1CNc2c(sc3ccc4nc(Oc5nc(Cl)ncc5CN5CCC(O)CC5)ccc4c23)C(=O)N1] |
| I-129 | 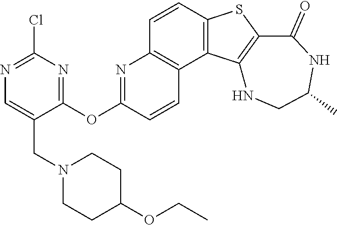 [SMILES: CCOC1CCN(Cc2cnc(Cl)nc2Oc2ccc3c(ccc4sc5c(c43)NC[C@@H](C)NC5=O)n2)CC1] |
| I-130 | 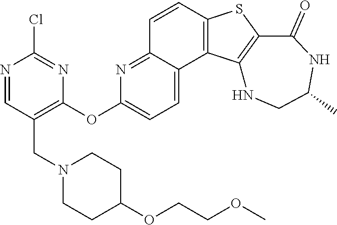 [SMILES: COCCOC1CCN(Cc2cnc(Cl)nc2Oc2ccc3c(ccc4sc5c(c43)NC[C@@H](C)NC5=O)n2)CC1] |
| I-131 | 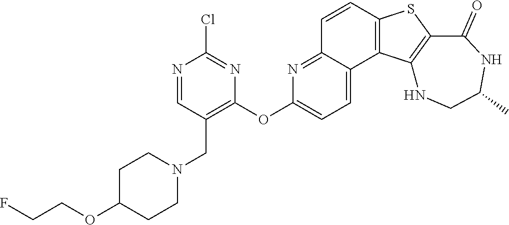 [SMILES: C[C@@H]1CNc2c(sc3ccc4nc(Oc5nc(Cl)ncc5CN5CCC(OCCF)CC5)ccc4c23)C(=O)N1] |
| I-132 | 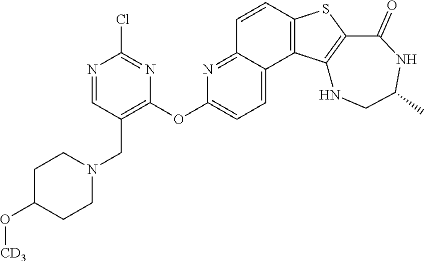 [SMILES: [2H]C([2H])([2H])OC1CCN(Cc2cnc(Cl)nc2Oc2ccc3c(ccc4sc5c(c43)NC[C@@H](C)NC5=O)n2)CC1] |
| I-133 | 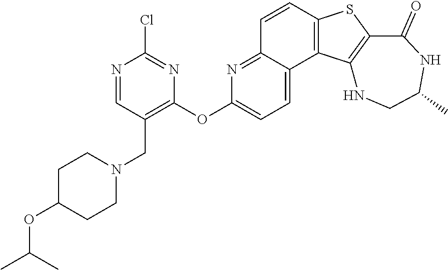 [SMILES: CC(C)OC1CCN(Cc2cnc(Cl)nc2Oc2ccc3c(ccc4sc5c(c43)NC[C@@H](C)NC5=O)n2)CC1] |
| I-134 | 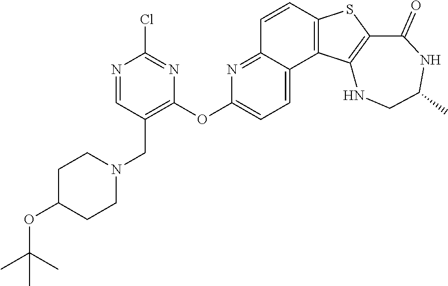 [SMILES: C[C@@H]1CNc2c(sc3ccc4nc(Oc5nc(Cl)ncc5CN5CCC(OC(C)(C)C)CC5)ccc4c23)C(=O)N1] |
| I-135 | 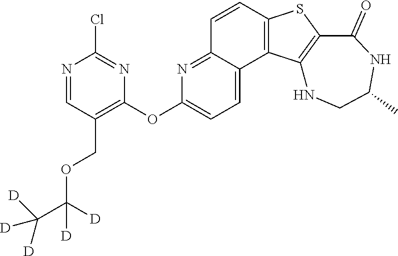 [SMILES: [2H]C([2H])([2H])C([2H])([2H])OCc1cnc(Cl)nc1Oc1ccc2c(ccc3sc4c(c32)NC[C@@H](C)NC4=O)n1] |

In some embodiments, the present disclosure provides a compound depicted in Table 1, or a pharmaceutically acceptable salt thereof.

It will be appreciated that, while compounds of formula I exist as a single stereoisomer, a person of ordinary skill in the art will recognize there is an enantiomeric form, as well as mixtures of both stereoisomers, e.g., a racemic or non-racemic mixture. The embodiments described above and herein also apply to the enantiomeric form of compounds of formula I, as well as mixtures of both stereoisomers, including a racemic or non-racemic mixture. A person skilled in the art can readily identify such enantiomeric compounds, as well as racemic and/or non-racemic mixtures of such enantiomers.

It will be appreciated that compounds of formula I are irreversible inhibitors of MK2 kinase. Without wishing to be bound by any particular theory, it is believed that compounds of formula I comprise a moiety capable of covalently binding to a key cysteine residue in the binding domain of MK2 kinase. Such a moiety is referred to herein as a “reactive moiety.” One of ordinary skill in the art will appreciate that MK2 kinase, and mutants thereof, have a cysteine residue in the binding domain. Without wishing to be bound by any particular theory, it is believed that proximity of a reactive moiety, present on a provided MK2 inhibitor, to the cysteine of interest facilitates covalent modification of that cysteine by the reactive moiety.

The cysteine residues of interest can also be described by an identifying portion of the amino acid sequence of MK2 kinase which includes the cysteine of interest. Thus, in certain embodiments, Cys140 of MK2 is characterized in that Cys140 is the cysteine embedded in the following amino acid sequence of MK2:

SEQ ID NO. 1:
MLSNSQGQSPPVPFPAPAPPPQPPTPALPHPPAQP
PPPPPQQFPQFHVKSGLQIKKNAIIDDYKVTSQVL
GLGINGKVLQIFNKRTQEKFALKMLQDCPKARREV
ELHWRASQCPHIVRIVDVYENLYAGRKCLLIVMEC
LDGGELFSRIQDRGDQAFTEREASEIMKSIGEAIQ
YLHSINIAHRDVKPENLLYTSKRPNAILKLTDFGF
AKETTSHNSLTTPCYTPYYVAPEVLGPEKYDKSCD
MWSLGVIMYILLCGYPPFYSNHGLAISPGMKTRIR
MGQYEFPNPEWSEVSEEVKMLIRNLLKTEPTQRMT
ITEFMNHPWIMQSTKVPQTPLHTSRVLKEDKERWE
DVKEEMTSALATMRVDYEQIKIKKIEDASNPLLLK
RRKKARALEAAALAH.

For the purpose of clarity, Cys140 is provided in the abbreviated amino acid sequence below:

SEQ ID NO. 2:
NLYAGRKCLLIVMEC(140)LDGGELFSRIQDR.

In both SEQ ID NOS. 1 and 2, Cysteine 140 is highlighted in bold with underlining.

In some embodiments, compounds of formula I include a reactive moiety characterized in that provided compounds covalently modify Cys140 of MK2.

In certain embodiments, compounds of formula I include a reactive moiety characterized in that a compound covalently modifies a target of Cys140 of MK2, thereby irreversibly inhibiting the kinase.

Thus, in some embodiments, a reactive moiety present on a provided MK2 inhibitor compound of formula I is capable of covalently binding to a cysteine residue thereby irreversibly inhibiting the enzyme. In some embodiments, the cysteine residue is Cys140 of MK2. One of ordinary skill in the art will recognize that a variety of reactive moieties, as defined herein, are suitable for such covalent bonding. Such reactive moieties include, but are not limited to, those described herein and depicted infra.

Methods of Treating a Disease or Disorder Associated with a Coronavirus

In some embodiments, the present disclosure methods of treating, stabilizing, or lessening the severity or progression of a respiratory disease or disorder. In some embodiments, the respiratory disease or disorder is a coronavirus. In some such embodiments, the coronavirus is SARS-CoV-2. In some embodiments, the present disclosure provides a method of treating a respiratory disease or disorder associated with SARS-CoV-2. In some such embodiments, the disease or disorder associated with SARS-CoV-2 is COVID-19.

In some embodiments, provided methods of treating, stabilizing, or lessening the severity of a respiratory disease or disorder (e.g., a disease or disorder associated with a coronavirus such as SARS-CoV-2; e.g., COVID-19) comprise administering to a patient a compound of formula I:

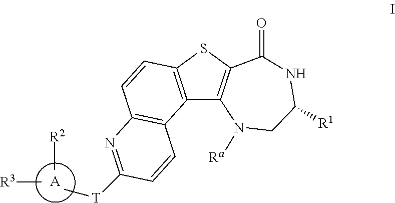

or a pharmaceutically acceptable salt thereof, wherein each of R¹, R², R³, T, Ring A, and T is as defined above and described herein.

In some embodiments, provided methods of treating, stabilizing, or lessening the severity of a respiratory disease or disorder (e.g., a disease or disorder associated with a coronavirus such as SARS-CoV-2; e.g., COVID-19) comprise administering to a patient compound I-82:

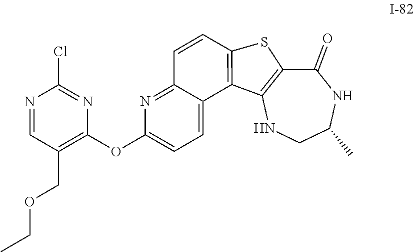 [SMILES: CCOCc1cnc(Cl)nc1Oc1ccc2c(ccc3sc4c(c32)NC[C@@H](C)NC4=O)n1]

or a pharmaceutically acceptable salt thereof.

In some embodiments, provided methods of treating, stabilizing, or lessening the severity of a respiratory disease or disorder (e.g., a disease or disorder associated with a coronavirus such as SARS-CoV-2; e.g., COVID-19) comprise administering to a patient compound I-100:

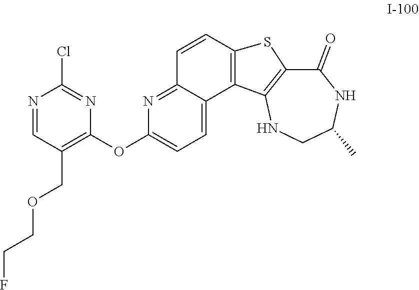 [SMILES: C[C@@H]1CNc2c(sc3ccc4nc(Oc5nc(Cl)ncc5COCCF)ccc4c23)C(=O)N1]

or a pharmaceutically acceptable salt thereof.

In some embodiments, the present disclosure provides methods of reducing the percentage of hospitalized patients requiring critical care and/or mechanical ventilation, as compared to a reference standard, in a patient suffering from or diagnosed with a respiratory disease or disorder (e.g., a disease or disorder associated with a coronavirus such as SARS-CoV-2; e.g., COVID-19). In some embodiments, a reference standard is the percentage of hospitalized patients requiring critical care and/or mechanical ventilation who have not been treated with a compound disclosed herein. In some embodiments, the present disclosure provides a method of reducing the frequency of respiratory progression characterized by increased oxygen requirement, as compared to a reference standard, in a patient suffering from or diagnosed with a respiratory disease or disorder (e.g., a disease or disorder associated with a coronavirus such as SARS-CoV-2; e.g., COVID-19). In some embodiments, a reference standard is respiratory progression characterized by increased oxygen requirement in patients who have not been treated with a compound disclosed herein.

In some embodiments, the present disclosure provides a method of reducing the frequency and duration of SARS-CoV-2 viral shedding in the upper respiratory tract, as compared with a reference standard, in a patient suffering from or diagnosed with a respiratory disease or disorder (e.g., a disease or disorder associated with a coronavirus such as SARS-CoV-2; e.g., COVID-19). In some embodiments, a reference standard is the quantity or amount of viral shedding in the upper respiratory tract in patients who have not been treated with a compound disclosed herein.

In some embodiments, the present disclosure provides a method of improving overall survival in a patient suffering from or diagnosed with a disease or disorder associated with a coronavirus, such as SARS-CoV-2 (e.g., COVID-19). In some embodiments, the overall survival is improved relative to a patient exhibiting one or more of the following characteristics: admitted to an intensive care unit, requires mechanical ventilation, suffers from sepsis or organ failure, develops respiratory failure, and/or otherwise dies due to the COVID-19 related disease. In some embodiments, overall survival is improved when a patient that is or has been discharged from an intensive care unit 14 days or less post administration of a therapy described herein. In some embodiments, overall survival is improved when a patient does not suffer respiratory failure 14 days or less post administration of a therapy described herein. In some embodiments, overall survival is improved when a patient does not die 14 days or less post administration of a therapy described herein. In some embodiments, overall survival is improved when a patient that is or has been discharged from an intensive care unit 28 days or less post administration of a therapy described herein. In some embodiments, overall survival is improved when a patient does not suffer respiratory failure 28 days or less post administration of a therapy described herein. In some embodiments, overall survival is improved when a patient does not die 28 days or less post administration of a therapy described herein. In some embodiments, overall survival is improved when a patient does not die 60 days or less post administration of a therapy described herein.

In some embodiments, the present disclosure provides a method of improving a National Early Warning Score 2 (NEWS 2) in a patient suffering from or diagnosed with a disease or disorder associated with a coronavirus, such as SARS-CoV-2 (e.g., COVID-19). In some embodiments, an improvement of NEWS 2 is determined by comparison to a baseline. In some embodiments, an improvement of NEWS 2 is determined by one or more of respiration rate, oxygen saturation, systolic blood pressure, pulse rate, level of consciousness or confusion, temperature.

In some embodiments, the present disclosure provides a method for decreasing the requirement of respiratory progression in a patient suffering from or diagnosed with a disease or disorder associated with a coronavirus, such as SARS-CoV-2 (e.g., COVID-19). In some embodiments, the decreased requirement is measured by a reduction in the need for additional oxygen or need for advanced ventilator support.

In some embodiments, the present disclosure provides a method for reducing frequency and duration of SARS-CoV-2 viral shedding in the upper respiratory tract (URT) of a patient suffering from or diagnosed with a disease or disorder associated with a coronavirus, such as SARS-CoV-2 (e.g., COVID-19).

In some embodiments, the present disclosure provides a method for modulating biomarkers associated with COVID-19 disease progression. In some embodiments, the biomarkers are selected from CRP, TNFα, IL-6, ferritin, LDH, and D-dimer. In some embodiments, the biomarkers are IL-6, IL-2, IL-7, GM-CSF, IP-10, MCP-1, MIP-1α, TNF-α, IL-1β, IL-8, IFN-α, IFN-β and IFN-γ. In some embodiments, a biomarker is TNF-α. In some embodiments, a biomarker is IL-6. In some embodiments, a biomarker is IL-10. In some embodiments, a biomarker is GM-CSF. In some embodiments, a biomarker is IL-1β. In some embodiments, a biomarker is IFN-γ.

In some embodiments, the present disclosure provides methods for treating a patient suffering from or diagnosed with a disease or disorder associated with a coronavirus (e.g., COVID-19), wherein the patient exhibits abnormalities relative to a subject not suffering from or diagnosed with the disease or disorder associated with a coronavirus, wherein the abnormalities are selected from one or more of leukopenia, hyperferritinemia, and elevation of one or more biomarkers selected from CRP, LDH, and IL-6.

In some embodiments, the present disclosure provides a method of treating, preventing, or lessening the severity of one or more symptoms associated with SARS-CoV-2 infection, the method comprising administering to a patient In need thereof a compound of formula I, or a pharmaceutically acceptable salt thereof. In some embodiments, the one or more symptoms associated with SARS-CoV-2 are selected from cytokine storm, hyperinflammation, hyperactivity of MK2, or a mutant thereof, pulmonary fibrosis, tissue injury, platelet activation, fibroblast invasion or infiltration, elevated thrombosis, lung epithelial damage, and abnormal monocyte macrophage cell infiltration. In some embodiments, the one or more symptoms associated with SARS-CoV-2 infection is increased or elevated levels of one or more of IL-1β, IL-1Rα, IL-7, IL-8, IL-9, IL-10, basic FGF (bFGF), GCSF, GM-CSF, IFNγ, IP10, MCP1, MIP1A, MIP1B, PDGF, TNF-α, and VEGF, as compared with a reference standard. In some embodiments, a reference standard is the level of the corresponding one or more of IL-1β, IL-1R^{a}, IL-7, IL-8, IL-9, IL-10, basic FGF (bFGF), GCSF, GM-CSF, IFNγ, IP10, MCP1, MIP1A, MIP1B, PDGF, TNF-α, and VEGF in patients who have not been treated with a compound disclosed herein.

In some embodiments, the one or more symptoms associated with SARS-CoV-2 infection is increased or elevated levels of one or more of IL-1β, TNF-α, IL-6, IL-10, GM-CSF, and MCP-1, as compared with a reference standard. In some embodiments, a reference standard is the level of the corresponding one or more of IL-1β, TNF-α, IL-6, IL-10, GM-CSF, and MCP-1 in patients who have not been treated with a compound disclosed herein.

In some embodiments, the one or more symptoms associated with SARS-CoV-2 infection is increased or elevated levels of one or more IL-2, IL-7, IL-10, GMCSF, IL-6, IP10, MCP1, MIP1A, and TNF-α, as compared with a reference standard. In some such embodiments, the patient is or has been admitted to the intensive care unit (ICU). In some embodiments, a reference standard is the level of the corresponding one or more of IL-2, IL-7, IL-10, GMCSF, IL-6, IP10, MCP1, MIP1A, and TNF-α in patients who have not been treated with a compound disclosed herein.

In some embodiments, the present disclosure provides a method of reducing a viral load of SARS-CoV-2 in a patient suffering from or diagnosed with a disease or disorder associated with a coronavirus, such as SARS-CoV-2 (e.g., COVID-19). In some embodiments, the method of reducing the viral load is determined by a RT-PCR negative result for the presence of SARS-CoV-2 in the upper respiratory tract (URT), or, as a change from baseline the viral load/titer (quantitatively).

In some embodiments, the present disclosure provides methods of treating, stabilizing, or lessening the severity or progression of a cytokine storm associated with or arising from a disease or disorder associated with a coronavirus (e.g., COVID-19). In some embodiments, a cytokine storm comprises elevation of a cytokine selected from the group consisting of IL-1β, TNF-α, IL-6, IL-10, GM-CSF, and MCP-1, or combinations thereof. In some embodiments, the present disclosure provides methods of treating, stabilizing, or lessening the severity or progression of hyperinflammation associated with or arising from a disease or disorder associated with a coronavirus (e.g., COVID-19). In some embodiments, the present disclosure provides methods of treating, stabilizing, or lessening the severity or progression of tissue injury associated with or arising from a disease or disorder associated with a coronavirus (e.g., COVID-19). In some embodiments, tissue injury comprises the circulatory system. In some embodiments, tissue injury comprises injury to lung tissue. In some embodiments, tissue injury comprises injury to heart tissue. In some embodiments, the present disclosure provides methods of destabilizing a cytokine or chemokine mRNA. In some embodiments, destabilization of a cytokine or chemokine mRNA is observed through a reduction in the mRNA (e.g., as measured by RT-PCR) in a patient suffering from or diagnosed with the disease or disorder associated with a coronavirus (e.g., COVID-19), as compared to a reference standard. In some embodiments, destabilization of a cytokine or chemokine mRNA is observed through a reduction in the corresponding cytokine or chemokine in a patient suffering from or diagnosed with the disease or disorder associated with a coronavirus (e.g., COVID-19). as compared to a reference standard. In some embodiments, a reference standard is a patient not suffering from or diagnosed with the disease or disorder associated with a coronavirus (e.g., COVID-19). In some embodiments, a cytokine or chemokine is selected from the group consisting of IL-1β, TNF-α, IL-6, IL-10, GM-CSF, and MCP-1, or combinations thereof.

In some embodiments, the present disclosure provides methods of treating, stabilizing, or lessening the severity or progression of cytopathic effects associated with a disease or disorder associated with a coronavirus, such as SARS-CoV-2 (e.g., COVID-19). In some embodiments, a cytopathic effect comprises or results in apoptosis. In some embodiments, a cytopathic effect comprises or results in cytoskeleton abnormalities, e.g., those associated with actin. In some embodiments, the present disclosure provides methods of treating, stabilizing, or lessening the severity or progression of viral proliferation in cells infected with a coronavirus (e.g., SARS-CoV-2).

In some embodiments, the present disclosure provides methods of treating, stabilizing, or lessening the severity or progression of pulmonary fibrosis associated with a disease or disorder associated with a coronavirus (e.g., COVID-19).

In some embodiments, the present disclosure provides a methods of treating, stabilizing, or lessening the severity or progression of a disease or disorder associated with a coronavirus, such as SARS-CoV-2 (e.g., COVID-19) in a patient in need thereof, wherein the patient exhibits cardiovascular risk factors, or cardiovascular or thrombotic complications. In some embodiments, a cardiovascular risk factor is high blood pressure, high blood cholesterol levels, diabetes mellitus, or obesity.

### Methods of Administering Compounds of Formula I

In some embodiments, provided methods comprise administering a compound of formula I (e.g., compound I-82) in an amount of about 3 mg to about 1000 mg. In some embodiments, a compound of formula I (e.g., compound I-82) is administered in an amount of about 3 mg to about 15 mg, about 10 mg to about 25 mg, about 15 mg to about 50 mg, about 25 mg to about 75 mg, about 50 mg to about 100 mg, about 75 mg to about 125 mg, about 100 mg to about 150 mg, or about 125 mg to about 200 mg.

In some embodiments, provided methods comprise administering a compound of formula I (e.g., compound I-82) in an amount of about 200 mg to about 300 mg, about 250 mg to about 500 mg, about 500 mg to about 750 mg, or about 750 mg to about 1000 mg.

In some embodiments, provided methods comprise administering to a patient in need thereof about 0.1 mg to about 10,000 mg of a compound of formula I (e.g., compound I-82). In some embodiments, provided methods comprise administering a compound of formula I (e.g., compound I-82) in an amount of about 0.1 mg to about 9000 mg, about 0.1 mg to about 8000 mg, about 0.1 to about 7000 mg, about 0.1 mg to about 6000 mg, about 0.1 mg to about 5000 mg, about 0.1 mg to about 4000 mg, about 0.1 mg to about 3000 mg, about 0.1 mg to about 2000, about 0.1 mg to about 1000 mg, about 0.1 mg to about 900 mg, about 0.1 mg to about 800 mg, about 0.1 mg to about 700 mg, about 0.1 mg to about 600 mg, about 0.1 mg to about 500 mg, about 0.1 mg to about 400 mg, about 0.1 mg to about 300 mg, about 0.1 mg to about 200 mg, about 0.1 mg to about 100 mg, about 0.1 mg to about 75 mg, about 0.1 mg to about 50 mg, about 0.1 mg to about 25 mg, about 0.1 mg to about 15 mg, about 0.1 mg to about 10 mg, about 0.1 mg to about 5 mg, about 0.1 mg to about 3 mg, or about 0.1 mg to about 1 mg.

In some embodiments, provided methods comprise administering a compound of formula I (e.g., compound I-82) in an amount of about 0.5 mg to about 500 mg, about 1 mg to about 400 mg, about 3 mg to about 300 mg, about 5 mg to about 200 mg, about 10 mg to about 150 mg, about 15 to about 100 mg, or about 25 mg to about 75 mg.

In some embodiments, provided methods comprise administering a compound of formula I (e.g., compound I-82) in an amount of about 1 mg to about 500 mg, about 3 mg to about 400 mg, about 5 mg to about 300 mg, about 10 mg to about 200 mg, about 15 mg to about 150 mg, about 25 mg to about 100 mg.

In some embodiments, provided methods comprise administering a compound of formula I (e.g., compound I-82) in an amount of about 3 mg to about 500 mg, about 5 mg to about 400 mg, about 10 mg to about 300 mg, about 15 mg to about 200 mg, about 25 mg to about 150 mg, about 50 mg to about 100 mg, or about 75 mg to about 125 mg.

In some embodiments, provided methods comprise administering a compound of formula I (e.g., compound I-82) in an amount of about 5 mg to about 500 mg, about 10 mg to about 400 mg, about 15 mg to about 300 mg, about 25 mg to about 200 mg, about 50 mg to about 150 mg, or about 75 mg to about 100 mg.

In some embodiments, provided methods comprise administering a compound of formula I (e.g., compound I-82) in an amount of about 10 mg to about 500 mg, about 15 mg to about 400 mg, about 25 mg to about 300 mg, about 50 mg to about 200 mg, about 75 mg to about 150 mg, or about 100 mg to about 125 mg.

In some embodiments, provided methods comprise administering a compound of formula I (e.g., compound I-82) in an amount of about 15 mg to about 500 mg, about 25 mg to about 400 mg, about 50 mg to about 300 mg, about 75 mg to about 200 mg, or about 100 to about 150 mg.

In some embodiments, provided methods comprise administering a compound of formula I (e.g., compound I-82) in an amount of about 25 mg to about 500 mg, about 50 mg to about 400 mg, about 75 mg to about 300 mg, about 100 to about 200 mg, or about 125 mg to about 150 mg.

In some embodiments, provided methods comprise administering a compound of formula I (e.g., compound I-82) in an amount of about 50 mg to about 500 mg, about 75 mg to about 400 mg, about 100 mg to about 300 mg, or about 150 mg to about 200 mg.

In some embodiments, provided methods comprise administering a compound of formula I (e.g., compound I-82) in an amount of about 75 mg to about 500 mg, about 100 mg to about 400 mg, or about 125 mg to about 300 mg.

In some embodiments, provided methods comprise administering a compound of formula I (e.g., compound I-82) in an amount of about 100 mg to about 500 mg, about 150 mg to about 400 mg, or about 200 mg to about 300 mg.

In some embodiments, provided methods comprise administering a compound of formula I (e.g., compound I-82) in an amount of about 200 mg to about 1000 mg, about 250 mg to about 900 mg, about 300 mg to about 800 mg, about 350 mg to about 750 mg, about 400 mg to about 700 mg, or about 500 mg to about 600 mg.

In some embodiments, the present invention provides methods of treating, stabilizing, or lessening the severity or progression of a disease or disorder associated with SARS-CoV-2 (e.g., COVID-19), wherein the method comprises administering to a patient in need thereof about 3 mg to about 400 mg of a compound of formula I (e.g., compound I-82). In some embodiments, the present invention provides methods of treating, stabilizing, or lessening the severity or progression of a disease or disorder associated with SARS-CoV-2 (e.g., COVID-19), wherein the method comprises administering to a patient in need thereof about 3 mg, about 10 mg, about 30 mg, about 100 mg, about 200 mg or about 400 mg of a compound of formula I (e.g., compound I-82).

In some embodiments, provided methods comprise administering a compound of formula I (e.g., compound I-82) in an amount of about 50 mg to about 150 mg. In some embodiments, provided methods comprise administering a compound of formula I (e.g., compound I-82) in an amount of about 60 mg to about 150 mg. In some embodiments, provided methods comprise administering a total daily dose a compound of formula I (e.g., compound I-82) in an amount of 54 mg to 66 mg. In some embodiments, provided methods comprise administering a total daily dose of a compound of formula I (e.g., compound I-82) in an amount of 56 mg to 64 mg. In some embodiments, provided methods comprise administering a total daily dose of a compound of formula I (e.g., compound I-82) in an amount of 58 mg to 62 mg. In some embodiments, provided methods comprise administering a total daily dose of a compound of formula I (e.g., compound I-82) in an amount of 135 mg to 165 mg. In some embodiments, provided methods comprise administering a total daily dose of a compound of formula I (e.g., compound I-82) in an amount of 140 mg to 160 mg. In some embodiments, provided methods comprise administering a total daily dose of a compound of formula I (e.g., compound I-82) in an amount of 145 mg to 155 mg. In some embodiments, provided methods comprise administering a total daily dose of a compound of formula I (e.g., compound I-82) in an amount of 148 mg to 152 mg.

In some embodiments, provided methods comprise administering a compound of formula I (e.g., compound I-82) in an amount of about 0.1 mg, about 0.2 mg, about 0.3 mg, about 0.4 mg, about 0.5 mg, about 1 mg, about 3 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 405 mg, about 410 mg, about 415 mg, about 420 mg, about 425 mg, about 430 mg, about 435 mg, about 440 mg, about 445 mg, about 450 mg, about 455 mg, about 460 mg, about 465 mg, about 470 mg, about 475 mg, about 480 mg, about 485 mg, about 490 mg, about 495, about 500 mg, about 505 mg, about 510 mg, about 515 mg, about 520 mg, about 525 mg, about 530 mg, about 535 mg, about 540 mg, about 545 mg, about 550 mg, about 555 mg, about 560 mg, about 565 mg, about 570 mg, about 575 mg, about 580 mg, about 585 mg, about 590 mg, about 595 mg, about 600 mg, about 605 mg, about 610 mg, about 615 mg, about 620 mg, about 625 mg, about 630 mg, about 635 mg, about 640 mg, about 645 mg, about 650 mg, about 655 mg, about 660 mg, about 665 mg, about 670 mg, about 675 mg, about 680 mg, about 685 mg, about 690 mg, about 695 mg, about 700 mg, about 705 mg, about 710 mg, about 715 mg, about 720 mg, about 725 mg, about 730 mg, about 735 mg, about 740 mg, about 745 mg, about 750 mg, about 755 mg, about 760 mg, about 765 mg, about 770 mg, about 775 mg, about 780 mg, about 785 mg, about 790 mg, about 795 mg, about 800 mg, about 805 mg, about 810 mg, about 815 mg, about 820 mg, about 825 mg, about 830 mg, about 835 mg, about 840 mg, about 845 mg, about 850 mg, about 855 mg, about 860 mg, about 865 mg, about 870 mg, about 875 mg, about 880 mg, about 885 mg, about 890 mg, about 895 mg, about 900 mg, about 905 mg, about 910 mg, about 915 mg, about 920 mg, about 925 mg, about 930 mg, about 935 mg, about 940 mg, about 945 mg, about 950 mg, about 955 mg, about 960 mg, about 965 mg, about 970 mg, about 975 mg, about 980 mg, about 985 mg, about 990 mg, about 995 mg, or about 1000 mg.

In some embodiments, provided methods comprise administering a compound of formula I (e.g., compound I-82) in an amount of about 60 mg. In some embodiments, provided methods comprise administering a compound of formula I (e.g., compound I-82) in an amount of about 150 mg.

In some embodiments, provided methods comprise administering a total daily dose of a compound of formula I (e.g., compound I-82) in an amount of about 1 mg to about 5 mg, about 8 mg to about 12 mg, about 28 mg to about 32 mg, about 98 mg to about 102 mg, about 198 mg to about 202 mg, or about 398 mg to about 402 mg.

In some embodiments, provided methods comprise administering to a patient in need thereof a compound of formula I (e.g., compound I-82) in an amount that is equivalent to about 3 mg/kg, about 5 mg/kg, about 10 mg/kg, about 15 mg/kg, or about 30 mg/kg in a mouse. In some embodiments, provided methods comprise administering to a patient in need thereof a compound of formula I (e.g., compound I-82) in an amount that is equivalent to about 100 mg/kg in a mouse. In some such embodiments, the amount of a compound of formula I (e.g., compound I-82) is about 15 mg, about 25 mg, about 50 mg, about 75 mg, about 150 mg, or about 500 mg.

In some embodiments, provided methods comprise administering to a patient in need thereof a compound of formula I (e.g., compound I-82) in an amount that is equivalent to about 5 mg/kg, about 20 mg/kg, about 30 mg/kg, or about 100 mg/kg in a rat. In some such embodiments, the amount of a compound of formula I (e.g., compound I-82) is about 50 mg, about 200 mg, about 300 mg, or about 1000 mg.

In some embodiments, provided methods comprise administering to a patient in need thereof a compound of formula I (e.g., compound I-82) in an amount that is equivalent to about 5 mg/kg, about 50 mg/kg, about 150 mg/kg or about 375 mg/kg in a monkey. In some such embodiments, the amount of a compound of formula I (e.g., compound I-82) is about 100 mg, about 1000 mg, about 3000 mg, or about 7500 mg.

In some embodiments, the present invention provides a use of a compound of formula I (e.g., compound I-82) in the manufacture of a medicament for treating, stabilizing, or lessening the severity or progression of a disease or disorder associated with SARS-CoV-2 (e.g., COVID-19). In some embodiments, the present invention provides a use of a compound of formula I (e.g., compound I-82) for treating, stabilizing, or lessening the severity or progression of a disease or disorder associated with SARS-CoV-2 (e.g., COVID-19).

In some embodiments, a compound of formula I (e.g., compound I-82), or a pharmaceutically acceptable composition thereof, is administered once daily (“QD”). In some embodiments, a compound of formula I (e.g., compound I-82), or a pharmaceutically acceptable composition thereof, is administered twice daily (“BID”). In some embodiments, a compound of formula I (e.g., compound I-82), or a pharmaceutically acceptable composition thereof, is administered three times a day (“TID”). In some embodiments, a compound of formula I (e.g., compound I-82), or a pharmaceutically acceptable composition thereof, is administered four times a day (“QID”).

In some embodiments, a compound of formula I (e.g., compound I-82), or a pharmaceutically acceptable composition thereof, is administered once weekly (“QW”).

In some embodiments, provided methods comprise administering a compound of formula I (e.g., compound I-82), or a pharmaceutically acceptable composition thereof, once daily for a period of 1, 2, 3, 4, 5, 6, 7, 8, 9, 10, 11, 12, 13, 14, 15, 16, 17, 18, 19, 20, 21, 22, 23, 24, 25, 26, 27 or 28 days. In some embodiments, a pharmaceutically acceptable composition comprising a compound of formula I (e.g., compound I-82), or a pharmaceutically acceptable composition thereof, is administered once daily for 14 consecutive days (“a 14-day cycle”). In some embodiments, a pharmaceutically acceptable composition comprising a compound of formula I (e.g., compound I-82), or a pharmaceutically acceptable composition thereof, is administered once daily for at least one 14-day cycle.

In some embodiments, provided methods comprise administering to a patient in need thereof a compound of formula I (e.g., compound I-82), or a pharmaceutically acceptable composition thereof, wherein the patient has failed at least one prior therapy.

In some embodiments, a compound of formula I (e.g., compound I-82) is administered according to a regimen that minimizes exposure to ultraviolet light. In some such embodiments, a compound of formula I (e.g., compound I-82) is administered according to a regimen that minimizes exposure to sunlight. In some embodiments, a compound of formula I (e.g., compound I-82) is administered according to a regimen that minimizes exposure to long wave ultraviolet A (UVA) radiation and/or short wave ultraviolet B radiation (UVB). In some embodiments, a compound of formula I (e.g., compound I-82) is administered in the evening.

In some embodiments, a patient to whom a compound of formula I (e.g., compound I-82) is administered is male. In some embodiments, a patient to whom a compound of formula I (e.g., compound I-82) is administered is a patient with one or more co-morbidities such as cardiovascular disease, cerebrovascular disease, and diabetes. In some embodiments, a patient to whom a compound of formula I (e.g., compound I-82) is administered is a patient who is ≥50 years old, ≥60 years old, ≥70 years old, or ≥80 years old.

In some embodiments, a patient to whom a compound of formula I (e.g., compound I-82) is administered requires oxygen support. In some embodiments, a patient to whom a compound of formula I (e.g., compound I-82) is administered requires ventilatory support. In some embodiments, a patient to whom a compound of formula I (e.g., compound I-82) is administered requires admission to an intensive care unit (ICU). In some embodiments, a patient to whom a compound of formula I (e.g., compound I-82) is administered has one or more cardiovascular risk factors. In some embodiments, a patient to whom a compound of formula I (e.g., compound I-82) is administered has one or more thrombotic complications.

### Formulations of Compounds of Formula I

In some embodiments, the present invention provides methods of treating, stabilizing, or lessening the severity or progression of a disease or disorder associated with SARS-CoV-2 (e.g., COVID-19), wherein the method comprises administering to a patient in need thereof a pharmaceutically acceptable composition comprising a compound of formula I (e.g., compound I-82). In some embodiments, the pharmaceutically acceptable composition is in an oral dosage form. In some such embodiments, the pharmaceutically acceptable composition is in the form of a capsule.

In some embodiments, provided pharmaceutically acceptable compositions comprise a compound of formula I (e.g., compound I-82) and one or more pharmaceutically acceptable excipients, such as, for example, binders, diluents, disintegrants, wetting agents, lubricants and adsorbents.

Pharmaceutical compositions for use in the present invention may comprise one or more binders. Binders are used in the formulation of solid oral dosage forms to hold the active pharmaceutical ingredient and inactive ingredients together in a cohesive mix. In some embodiments, pharmaceutical compositions of the present invention comprise about 5% to about 50% (w/w) of one or more binders and/or diluents. In some embodiments, pharmaceutical compositions of the present invention comprise about 20% (w/w) of one or more binders and/or diluents. Suitable binders and/or diluents (also referred to as “fillers”) are known in the art. Representative binders and/or diluents include, but are not limited to, starches such as celluloses (low molecular weight HPC (hydroxypropyl cellulose), microcrystalline cellulose (e.g., Avicel©), low molecular weight HPMC (hydroxypropyl methylcellulose), low molecular weight carboxymethyl cellulose, ethylcellulose), sugars such as lactose (i.e. lactose monohydrate), sucrose, dextrose, fructose, maltose, glucose, and polyols such as sorbitol, mannitol, lactitol, maltitol and xylitol, or a combination thereof. In some embodiments, a provided composition comprises a binder of microcrystalline cellulose and/or lactose monohydrate.

Pharmaceutical compositions for use in the present invention may further comprise one or more disintegrants. Suitable disintegrants are known in the art and include, but are not limited to, agar, calcium carbonate, sodium carbonate, sodium bicarbonate, cross-linked sodium carboxymethyl cellulose (croscarmellose sodium), sodium carboxymethyl starch (sodium starch glycolate), microcrystalline cellulose, or a combination thereof. In some embodiments, provided formulations comprise from about 1%, to about 25% disintegrant, based upon total weight of the formulation.

Wetting agents, also referred to as bioavailability enhancers, are well known in the art and typically facilitate drug release and absorption by enhancing the solubility of poorly-soluble drugs. Representative wetting agents include, but are not limited to, poloxamers, polyoxyethylene ethers, polyoxyethylene fatty acid esters, polyethylene glycol fatty acid esters, polyoxyethylene hydrogenated castor oil, polyoxyethylene alkyl ether, polysorbates, and combinations thereof. In certain embodiments, the wetting agent is a poloxamer. In some such embodiments, the poloxamer is poloxamer 407. In some embodiments, compositions for use in the present invention comprise from about 1% to about 30% by weight of wetting agent, based upon total weight of the blended powder.

Pharmaceutical compositions of the present invention may further comprise one or more lubricants. Lubricants are agents added in small quantities to formulations to improve certain processing characteristics. Lubricants prevent the formulation mixture from sticking to the compression machinery and enhance product flow by reducing interparticulate friction. Representative lubricants include, but are not limited to, magnesium stearate, glyceryl behenate, sodium stearyl fumarate and fatty acids (i.e. palmitic and stearic acids). In certain embodiments, a lubricant is magnesium stearate. In some embodiments, provided formulations comprise from about 0.2% to about 3% lubricant, based upon total weight of given formulation.

Pharmaceutical compositions of the present invention may further comprise one or more adsorbents. Representative adsorbents include, but are not limited to, silicas (i.e. fumed silica), microcrystalline celluloses, starches (i.e. corn starch) and carbonates (i.e. calcium carbonate and magnesium carbonate). In some embodiments, provided formulations comprise from about 0.2% to about 3% adsorbent, based upon total weight of given formulation.

In some embodiments, the present methods comprising administering a pharmaceutical composition comprising a compound of formula I (e.g., compound I-82), methyl cellulose and Tween 80. In some such embodiments, the pharmaceutical composition is a spray-dried dispersion (SDD).

In some embodiments, provided pharmaceutical compositions comprise a compound of formula I (e.g., compound I-82), HPMCAS, microcrystalline cellulose, croscarmellose sodium, silicon dioxide, and magnesium stearate.

In some embodiments, provided pharmaceutical compositions comprise a unit dose of a compound of formula I (e.g., compound I-82). A person of ordinary skill will appreciate that the unit dosage forms described herein refer to an amount of a compound of formula I (e.g., compound I-82) as a free base. A person skilled in the art will further appreciate that, when a pharmaceutical composition comprises a salt form of a compound of formula I (e.g., compound I-82), the amount of the salt form present in the composition is an amount that is equivalent to a unit dose of the free base of a compound of formula I (e.g., compound I-82). In some embodiments, a unit dose comprises about 3 mg, about 10 mg, about 15 mg, about 25 mg, about 50 mg, about 75 mg, about 100 mg, about 125 mg, about 150 mg, about 175 mg, or about 200 mg of a compound of formula I (e.g., compound I-82). In some embodiments, a unit dose comprises about 3 mg, about 5 mg, about 10 mg, about 15 mg, about 25 mg, about 50 mg, about 75 mg, or about 100 mg of a compound of formula I (e.g., compound I-82). In some embodiments, a unit dose of a compound of formula I (e.g., compound I-82) comprises about 30 mg. In some embodiments, a unit dose of a compound of formula I (e.g., compound I-82) comprises about 60 mg. In some embodiments, a unit dose of a compound of formula I (e.g., compound I-82) comprises about 150 mg.

In some embodiments, the present invention provides methods of treating, stabilizing, or lessening the severity or progression of a disease or disorder associated with SARS-CoV-2 (e.g., COVID-19), wherein the method comprises administering to a patient in need thereof a unit dose of a compound of formula I (e.g., compound I-82), wherein the unit dose of a compound of formula I (e.g., compound I-82) is about 3 mg, about 5 mg, about 10 mg, about 15 mg, about 25 mg, about 50 mg, about 75 mg, or about 100 mg. In some embodiments, the present invention provides methods of treating, stabilizing or lessening the severity or progression of a disease or disorder associated with SARS-CoV-2 (e.g., COVID-19), wherein the method comprises administering to a patient in need thereof a unit dose of a compound of formula I (e.g., compound I-82), wherein the unit dose of Compound 1 is about 30 mg, about 60 mg, or about 150 mg.

### EXAMPLES

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein.

### Example 1

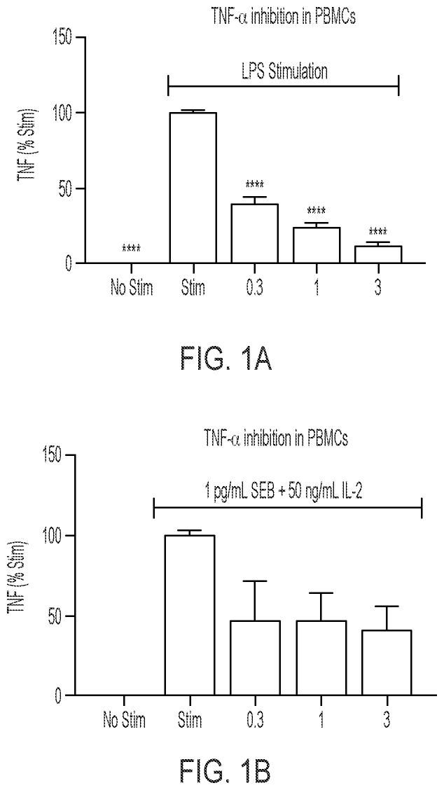

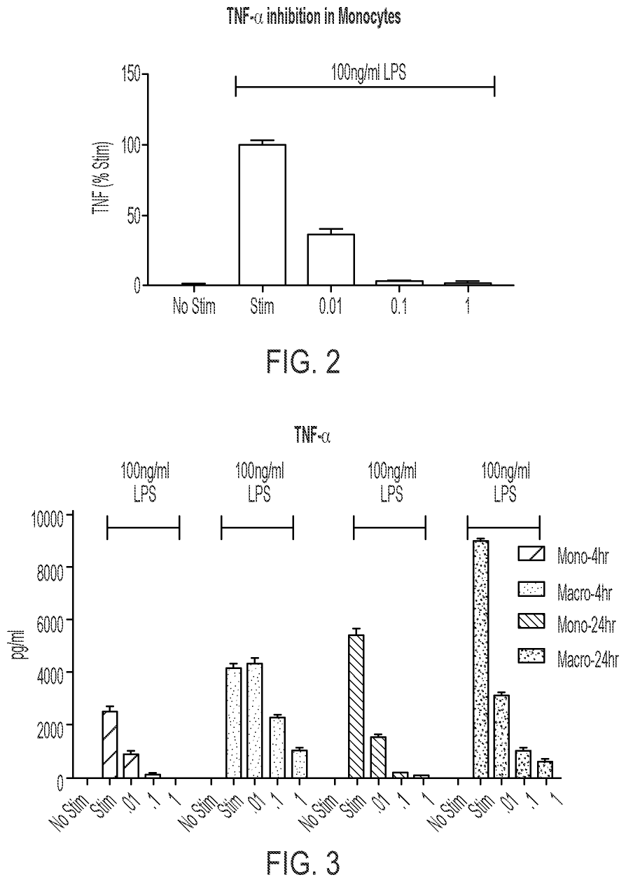

The present example demonstrates that, upon treating with compound I-82, TNF-α levels are decreased in a dose-dependent manner in peripheral blood mononuclear cell (PBMCs) (see FIGS. 1A and 1B) and monocytes (see FIG. 2).

Frozen human PBMCs from healthy donors were thawed and washed with PBS. Cells were pretreated with DMSO or compound I-82 for 1 hour, followed by LPS treatment for 24 hours or staphylococcal enterotoxin-B/interleukin-2 treatment for 3 days. All cells were cultured at 37° C. with 5% CO₂ in a humidified incubator. Culture media was harvested and frozen for cytokine and chemokine assessment and results are reported in FIG. 1A (LPS stimulation) and FIG. 1B (for SEB/IL-2 treatment).

In addition, PBMCs were isolated from buffy coat using Ficoll gradient method and were used for monocyte enrichment without CD16 depletion with the Stem Cell technologies kit (Cat #19058). Monocytes were pre-treated for 1 hr with DMSO or compound I-82 and then stimulated by LPS for 23 hours. All cells were cultured at 37° C. with 5% CO₂ in a humidified incubator. Supernatant was collected for cytokine and chemokine analysis and results are reported in FIG. 2.

### Example 2

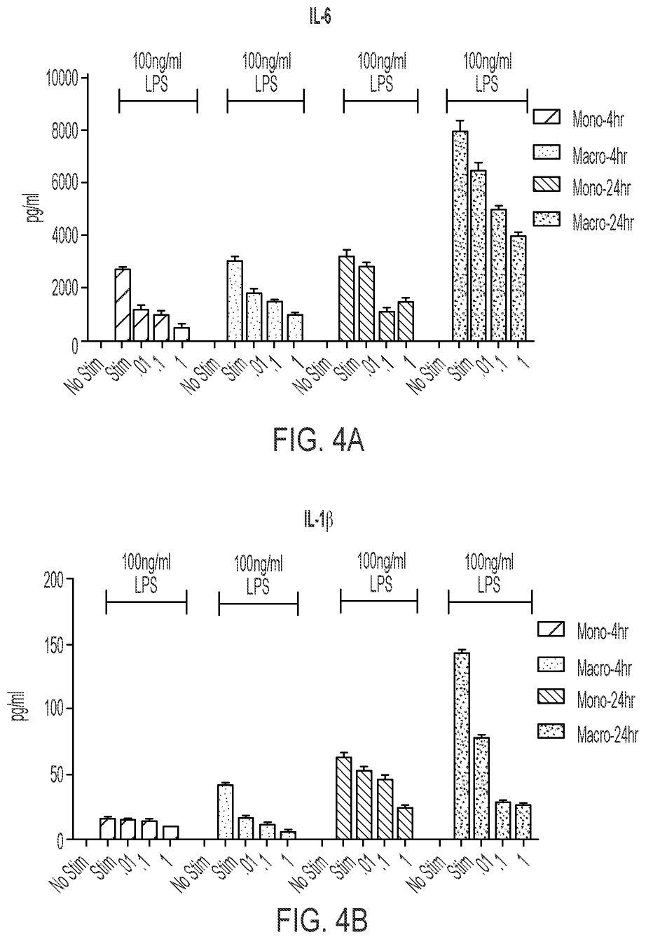

The present example demonstrates that a compound I-82 inhibits cytokine production in a dose-dependent manner in LPS-stimulated monocytes and macrophages. For example, the present example demonstrates that compound I-82 inhibits TNF-α, IL-6, and IL-1β in monocytes and macrophages (see FIGS. 3-4).

For Monocytes: PBMCs were isolated from buffy coat using Ficoll gradient method and were used for monocyte enrichment without CD16 depletion with the Stem Cell technologies kit (Cat #19058). Monocytes were pre-treated for 1 hr with DMSO or compound I-82 and then stimulated by LPS for 3 or 23 hours, for a total of 4 or 24 hours of MK2 inhibition. All cells were cultured at 37° C. with 5% CO₂ in a humidified incubator. Supernatant was collected for cytokine and chemokine analysis. Results are reported in FIGS. 3 and 4.

For Macrophages: monocytes were differentiated into macrophages by stimulation with 50 ng/ml of MCS-F for 5 days followed by treatment with 50 ng/ml Interferon gamma for ˜16 hours. Macrophages were then plated, allowed to acclimate for 24 hours. Effect of a compound I-82 on cytokine production was analyzed in monocytes or macrophages by pre-treatment for 1 hr with DMSO or compound I-82, followed by LPS stimulation (100 ng/ml) for 3 or 23 hrs, for a total of 4 or 24 hours of MK2 inhibition. All cells were cultured at 37° C. with 5% CO₂ in a humidified incubator. Supernatants were collected for further analysis. Cytokine and chemokine analysis was performed in the supernatants using Magpix magnetic beads. Results are reported in FIGS. 3 and 4.

### Example 3

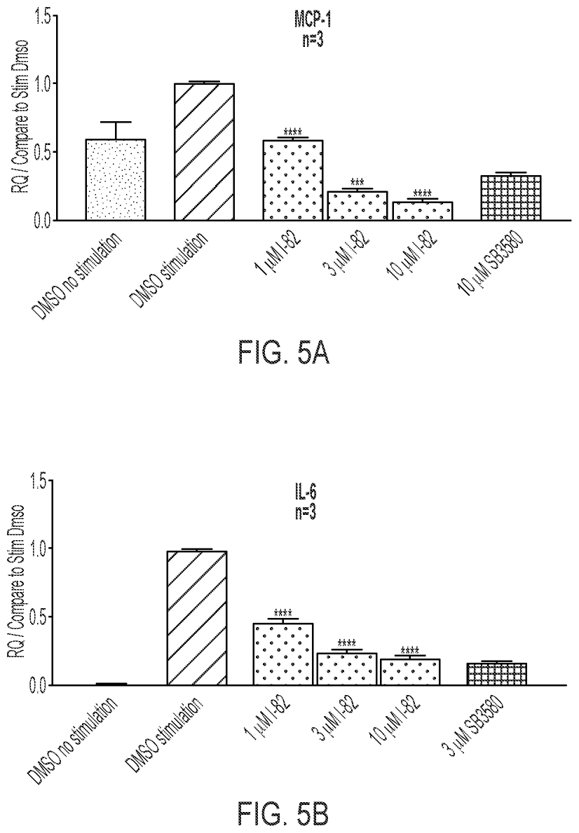

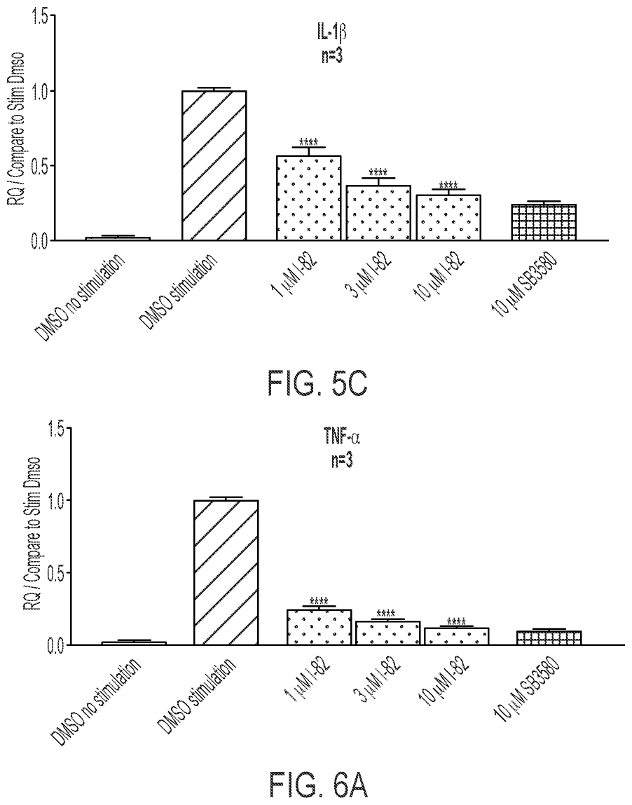

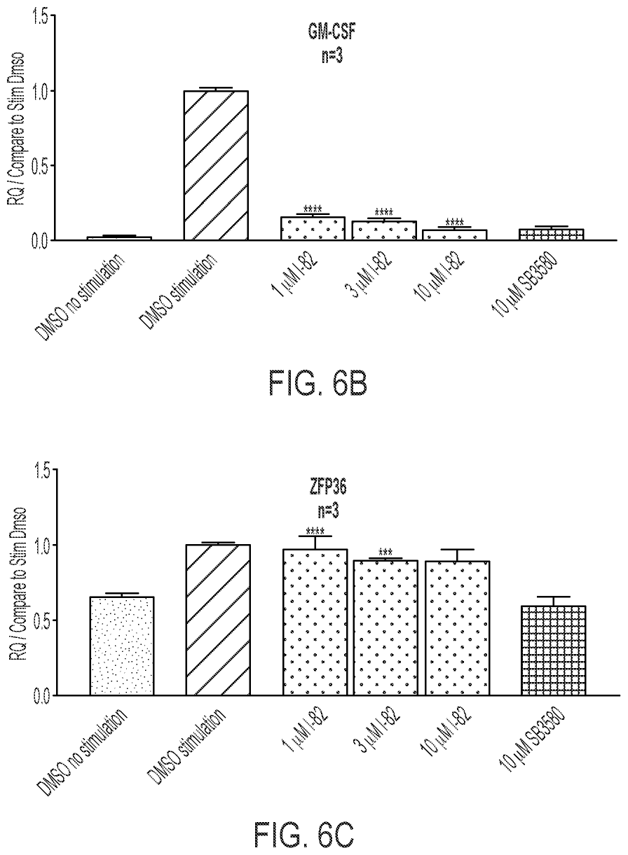

The present example demonstrates that compound I-82 inhibits gene expression of TNF-α, IL-6, MCP-1, GM-CSF, and IL-1β, but not TTP (ZFP36) (see FIGS. 5-6). PBMCs from HV were treated with LPS for 1.5 hrs followed by incubation with compound I-82 or p38 inhibitor SB203580 for 1.5 hrs. RNA was isolated from the PBMCs and RT-PCR was performed with probe sets for various cytokines and chemokines. Results are reported in FIGS. 5 and 6.

### Example 4

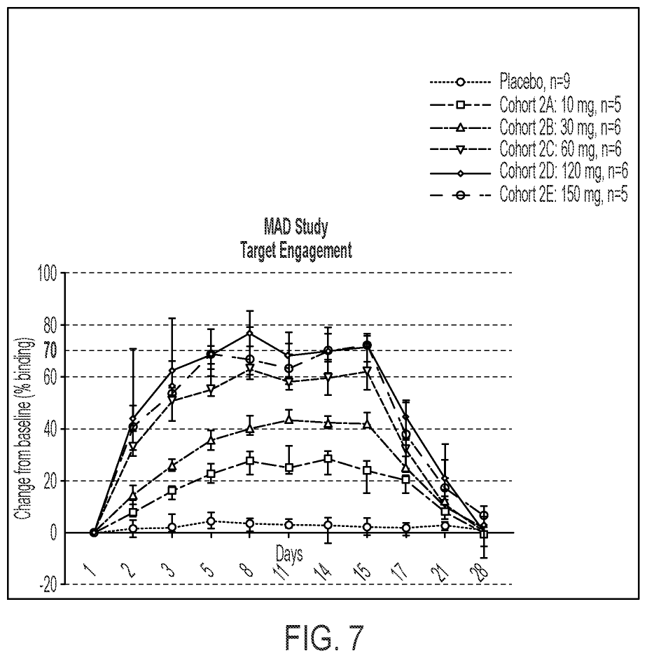

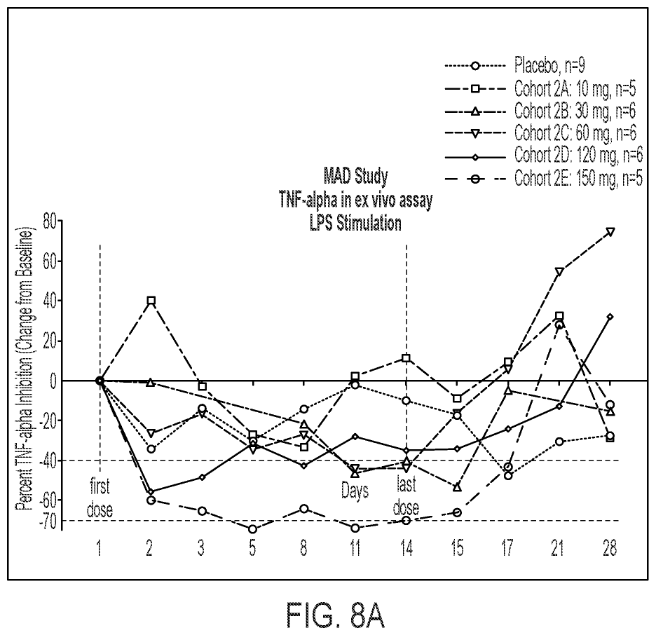

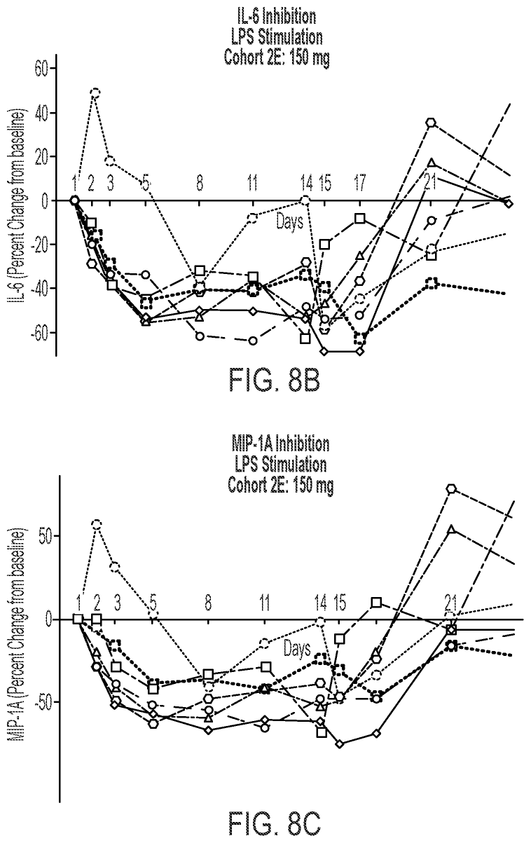

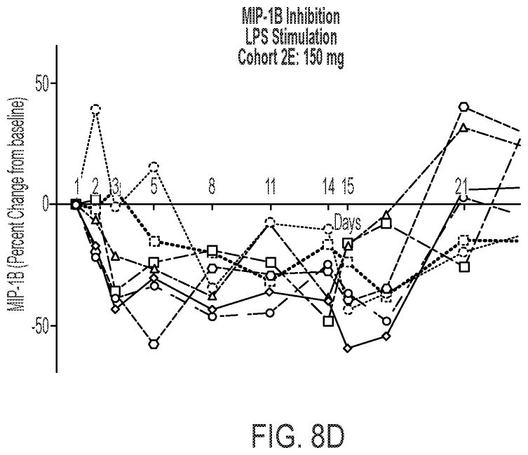

The present example demonstrates that increase in target engagement correlates with inhibition of TNF-α and other cytokines and chemokines (see FIGS. 7-8).

Multiple Ascending Dose (MAD) Study: a randomized, double-blind, placebo-controlled study to evaluate the safety, tolerability, PK, and PD of compound I-82 after administration of multiple oral doses (QD for 14 days) in healthy adult subjects. The study consisted of escalating multiple doses in sequential groups. Thirty-seven subjects were enrolled into 5 dose level cohorts (10, 30, 60, 120, and 150 mg). Each dose level cohort consisted of 6 to 8 subjects; 5 or 6 subjects received compound I-82 and 1 or 2 subjects received placebo according to the randomization schedule.

Each subject participated in a screening phase, a treatment phase (included baseline). Eligible subjects were admitted to the study site on Day −1 and resided at the study site from Day −1 to Day 17. The first dose of study drug was administered on Day 1, under fasted conditions, according to the randomization schedule. The same QD dose was administered for the remainder of the planned treatment schedule (Day 2 to Day 14, inclusive). For each dose level cohort in Part 2, blood samples were collected on Day 1 predose (0-hour), and predose (0-hour) on Days 2, 3, 5, 8, 11, and 14. Blood samples were also collected at approximately the same time of the day as the Days 1 to 14 samples on Days 15, 17, 21, and 28. Subjects were discharged from the study site on Day 17 upon satisfactory safety review and completion of the required study procedures. For all dose level cohorts, subjects returned to the study site for outpatient visits on Day 21 and Day 28. There were no early terminations.

Target Engagement in MAD Study: Whole blood samples were collected in CPT tubes from each subject on the days listed in the study protocol. PBMCs were isolated for target engagement at the clinical site. Target engagement of compound I-82 was measured using the Streptavidin Mass Shift (SMaSh) assay in peripheral blood mononuclear cells (PBMCs). Samples were evaluated for percent of MK2 bound to compound I-82 versus percent of free MK2 in PBMCs under the indicated cohort dosage conditions. Target engagement was measured by calculating the change in percent bound MK2 from baseline. Results are reported in FIG. 7.

Pharmacodynamic assessment in MAD Study: Effects of compound I-82 on innate cell activities were evaluated by assessing cytokine and chemokine production following lipopolysaccharide (LPS) ex vivo. Ex vivo stimulation was performed in whole blood using the TruCulture® assay system (Myriad RBM, Austin, Tex.). The effect of compound I-82 was determined on the production of cytokines and chemokines in a whole blood assay with LPS as stimulant using the TruCulture assay system (Myriad RBM, Austin, Tex.). Briefly, blood (1 ml) was drawn into the LPS-containing TruCulture tubes from each subject on the days listed in the study protocol. The truculture tubes were placed in a 37° C. block thermostat for 24±1 hrs and then frozen at −70° C. Media mixed with plasma was analyzed for TNF-α and other cytokine/chemokine levels. TNF-α inhibition in ex vivo LPS-stimulated blood was measured as percent change in TNF-α levels from baseline. Percent change in other cytokines/chemokines in the ex vivo stimulation assay was also measured with respect to their baseline levels. Results are reported in FIG. 8.

### Example 6

Protocol Summary. The study will enroll patients to receive a compound disclosed herein (e.g., a compound of formula I, such as compound I-82), also referred to as “study compound,” in addition to standard of care (SOC). The study population will consist of subject who are 18 years of age or older with laboratory (RT-PCR) confirmed infection with SARS-CoV-2, and who are hospitalized with moderate to severe symptoms including one or more of the following: SpO2≤93% on room air, PaO2/FiO2≤300 mmHg but not requiring mechanical ventilation, respiratory rate≥30 per minute, or positive chest CT or X-ray for pneumonia. Safety and preliminary efficacy will be assessed based on subjects enrolled with treatment for 14 days, and 7-day and 28-day follow-up visits. The primary endpoint will be determination of the proportion of subjects admitted to intensive care unit, mechanical ventilation, sepsis, organ failure, suffer respiratory failure, or death due to COVID-19 related disease.

Objectives. The primary objective of the study is to evaluate the efficacy of a study compound and standard of care (SOC) in reducing the percentage of hospitalized patients requiring critical care and/or mechanical ventilation. The secondary objectives are to evaluate:
Clinical status assessment using the 8-point ordinal scale
National Early Warning Score (NEWS) 2
Frequency of respiratory progression characterized by increased oxygen requirement or more advanced ventilator support.
Frequency and duration of SARS-CoV-2 viral shedding in upper respiratory tract specimen
Safety of a study compound in adult patients with COVID-19 respiratory disease
Frequency of serious adverse events
All cause mortality
Time to discharge
Modulation of biomarkers associated with COVID-19 disease progression (e.g., Cardiac Troponin, CPK, CRP, Ferritin, LDH, TNFα, IL-6, D-dimer, IL-6, TNFα, IFN-gamma, phospho-STAT3)
   The exploratory objectives are:
To evaluate the reduction in and/or clearance of SARS-CoV-2 viral titers and production of anti-SARS-CoV2 antibodies
To evaluate the pharmacodynamics as measured by changes in MK2 and p38 pathways and expression of downstream immunomodulatory and/or inflammatory cytokines
To evaluate molecular and cellular biomarkers which may be prognostic and/or predictive of response including but not limited to:
Flow cytometry for immune cell profiling
Genetic sequencing for predisposition variants including clonal hematopoiesis
Expression and activation of cellular proteins involved in viral entry and replication Study Endpoints are summarized in Table 2.

TABLE 2

| Endpoint | Name | Description | Timeframe |
|---|---|---|---|
| Primary | Critical care rate | Proportion of subjects with | From enrollment/ |
| | | respiratory failure (requiring | randomization until Day |
| | | invasive mechanical | 21 |
| | | ventilation), sepsis | |
| | | (requiring the use of | |
| | | vasopressors), organ failure, | |
| | | or death | |
| Secondary | Clinical status | Proportion of subjects with | From enrollment/ |
| | assessment | at least a 2-point | randomization until Day |
| | Proportion of subjects | improvement using the 8- | 21 |
| | | point ordinal scale | |
| | Clinical status | Time to improvement of at | From enrollment/ |
| | assessment | least 2 points using the 8- | randomization until Day |
| | Time to improvement | point ordinal scale | 21 |
| | National Early | Mean change from baseline | From enrollment/ |
| | Warning Score 2 | for total and individual | randomization until Day |
| | (NEWS2) | scores | 21 |
| | Mean change | | |
| | National Early | Time to the total score <2 | From enrollment/ |
| | Warning Score 2 | | randomization until Day |
| | (NEWS2) | | 21 |
| | Time to score <2 | | |
| | Time to hospital | Time (in days) until | From enrollment/ |
| | discharge | discharge from hospital | randomization until Day |
| | | | 44 |
| | Safety of study drug in | Incidence and severity of all | From ICF signature up |
| | adult patients with | Grade AEs based on | until 30 days after last |
| | COVID-19 respiratory | CTCAE | dose |
| | disease | Incidence and severity of | |
| | | Grade 3 and 4 AEs | |
| | | including laboratory | |
| | | parameters based on | |
| | | CTCAE | |
| | | Incidence of subjects with | |
| | | serious adverse events | |
| | All-cause mortality | Proportion of subjects who | From enrollment/ |
| | | died | randomization until Day |
| | | | 21 |
| | Modulation of | Change from baseline of | From enrollment/ |
| | biomarkers associated | biomarkers (e.g., Cardiac | randomization until Day |
| | with COVID-19 | Troponin, CPK, CRP, D- | 21 |
| | progression | dimer, ferritin, IL-6, LDH, | |
| | | and TNFα) | |
| Exploratory | SARS-CoV-2 reduction | Change from baseline in | From enrollment/ |
| | or clearance, and | viral load/titer | randomization until Day |
| | production of anti- | Change from baseline in | 21 |
| | SARS-CoV-2 | anti-SARS-CoV-2 | |
| | antibodies | antibodies | |
| | Pharmacodynamic | Changes in MK2-p38 | From enrollment/ |
| | biomarkers | pathway activation | randomization until Day |
| | | Changes in | 21 |
| | | immunomodulatory and/or | |
| | | inflammatory cytokines | |
| | Prognostic and/or | Flow cytometry for immune | From enrollment/ |
| | predictive biomarkers | cell profiling | randomization until Day |
| | | Genetic alterations | 21 |
| | | including variants | |
| | | associated with clonal | |
| | | hematopoiesis | |
| | | Changes in expression and | |
| | | activation of cellular | |
| | | proteins involved in viral | |
| | | entry and replication | |
| | | Characterization of SARS- | |
| | | CoV-2 DNA/RNA | |
| | Pharmacokinetics | Assessed by population PK | From enrollment/ |
| | | approach | randomization until Day |
| | | | 15 |

Study Design. This is an open-label, multicenter trial to evaluate the efficacy and safety of a study compound in symptomatic hospitalized patients with COVID-19 respiratory disease. The study consists of two phases: Part 1 Single Arm Phase and Part 2 Randomized Phase. Eligible subjects are hospitalized with a documented positive SARS-CoV-2 infection but not requiring invasive mechanical ventilation as well as one or more of the following respiratory parameters: peripheral oxygen saturation (SpO2)≤93% on room air; partial pressure of oxygen/fraction of inspired oxygen (PaO2/FiO2)≤300 mmHg based on available arterial blood gas (ABG) analysis; respiratory rate≥24 breaths per minute; or positive chest x-ray or computed tomography (CT) for pneumonia.

Part 1: Single Arm Phase. Eligible subjects (N=approximately 30) will be enrolled to receive a study compound plus SOC. The safety and preliminary efficacy will be assessed based on subjects enrolled with treatment for 14 days with treatment follow-up (Day 21, about 7 days post-last does of treatment) and safety follow up (Day 44, about 30 days post-last dose of treatment) visits. The data collected from this phase will be reviewed for recommendation to move into Part 2.

Part 2: Randomization Phase. Eligible subjects (N=100, 50 in each arm) will be randomized 1:1 to receive study drug plus SOC or SOC alone. Subjects will be stratified by age (<60 versus ≥60 years of age) and NEWS2 score (<5 versus ≥5). Safety and efficacy will be assessed for 14 days with treatment follow-up (Day 21, ˜7 days post-treatment) and safety follow-up (Day 44, ˜30 days post-treatment) visits.

Study Treatments. A study compound will be administered as 150 mg/day orally for up to 14 days during the inpatient stay given at the same time each day, preferably in the evening with a meal. However, a study compound may be taken with or without regard to food. Treatment with a study compound should be discontinued for Common Terminology Criteria for Adverse Events (CTCAE) Grade 4 or Grade 3 adverse events.

Study Population. Study population consist of subjects 18 years of age or older with laboratory (RT-PCR) confirmed infection with SARS-CoV-2 who are hospitalized with moderate to severe symptoms including one or more of the following: SpO2≤93% on room air; PaO2/FiO2≤300 mmHg but not requiring mechanical ventilation; Respiratory Rate≥30 per minute; or Positive Chest CT or X-ray for pneumonia. Approximately 125 subjects will be enrolled: 25 subjects will be enrolled in Part 1 single arm phase and 100 subjects in Part 2 randomization phase.

Inclusion Criteria. Subjects must satisfy the following criteria to be enrolled in the study:
1. Subject is at least 18 years of age at the time of signing the informed consent form (ICF)
2. Subject is confirmed positive for SARS-CoV-2 for not more than 14 days prior to enrollment
3. Subject is hospitalized, but not requiring invasive mechanical ventilation, with one or more of the following:
   a. SpO2≤93% on room air,
   b. PaO2/FiO2≤300 mmHg based on available ABG analysis,
   c. Respiratory Rate≥24 breaths per minute,
   d. Positive chest X-ray or CT for pneumonia within 3 days prior to enrollment (Part 1) or randomization (Part 2).
4. Subject must understand and voluntarily sign an ICF prior to any study-related assessments/procedures being conducted.
5. Subject is willing and able to adhere to the study visit schedule and other protocol requirements
6. A female of childbearing potential (FCBP) must:
   a. Have a negative pregnancy tests as verified by the Investigator prior to starting study therapy. She must agree to ongoing pregnancy testing during the course of the study, and after end of study therapy. This applies even if the subject practices true abstinence* from heterosexual contact.
   b. Either commit to true abstinence* from heterosexual contact (source documented) or agree to use, and be able to comply with highly effective contraception** without interruption, prior to starting investigational product, during the study therapy (including dose interruptions), and for 30 days after discontinuation of study therapy.
   Note: A female of childbearing potential (FCBP) is a female who: 1) has achieved menarche at some point, 2) has not undergone a hysterectomy or bilateral oophorectomy, or 3) has not been naturally postmenopausal (amenorrhea following cancer therapy does not rule out childbearing potential) for at least 24 consecutive months (ie, has had menses at any time in the preceding 24 consecutive months).
7. Male subjects must:
   a. Practice true abstinence* or agree to use a condom during sexual contact with a pregnant female or a female of childbearing potential while participating in the study, during dose interruptions and for at least 30 days following investigational product discontinuation, or longer if required for each compound and/or by local regulations, even if he has undergone a successful vasectomy. *True abstinence is acceptable when this is in line with the preferred and usual lifestyle of the subject. [Periodic abstinence (e.g., calendar, ovulation, symptothermal, post-ovulation methods) and withdrawal are not acceptable methods of contraception].**Agreement to use highly effective methods of contraception that alone or in combination result in a failure rate of a Pearl index of less than 1% per year when used consistently and correctly throughout the course of the study. Such methods include: Combined (estrogen and progestogen containing) hormonal contraception: Oral; Intravaginal; Transdermal; Progestogen-only hormonal contraception associated with inhibition of ovulation: Oral; Injectable hormonal contraception; Implantable hormonal contraception; Placement of an intrauterine device; Placement of an intrauterine hormone-releasing system; Bilateral tubal occlusion; Vasectomized partner.
8. Subjects agree to limit ultraviolet (UV) exposure during the study and for at least 3 days after final dose of study compound by adhering to the photoprotection guidelines below:
   a. Avoid being outdoors when the sun is at maximal intensity
   b. Wear clothing that would protect from the sun such as long-sleeves, sunglasses, and a hat
   c. Use sunscreen lotion in accordance with local guidelines

Exclusion Criteria. The presence of any of the following will exclude a subject from enrollment:

Disease Specific Requirements:
1. Subject has been hospitalized due to COVID-19 for more than 5 days.
2. Subject is receiving an anti-IL-6 treatment

Prior and/or Current Medications/Therapies:
1. Treatment with isoniazid within 4 weeks of the Baseline Visit and at any time during the Screening Period, up through the first dose of the study compound.
2. Use of any medications that are substrates of one or more of the transporters P-gp, BCRP, OCT1, OATP1B1, and OATP1B3 and have a narrow therapeutic index (e.g., methotrexate, sulfasalazine, and leflunomide).
   Note: At least 1-month washout period prior to randomization is required for the conventional synthetic disease-modifying antirheumatic drugs (DMARDs), except for leflunomide, which has to be discontinued for 8 weeks prior to randomization unless a cholestyramine washout has been performed. Subjects should not discontinue any of the above synthetic DMARDS for the sole purpose of participating in this trial.
3. Participation in any study of an investigational drug within one month or 5 PK or PD half-lives of the study compound, whichever is longer, prior to Screening Visit, or participation in more than one study with an investigational agent for COVID-19 within one year prior to Screening Visit

Permitted Concomitant Medications and Procedures.

Standard of care treatment according to site practice with non-investigational agents is permitted.

Standard of care therapy for COVID-19 is currently evolving and will follow emerging guidance documents (e.g., the “Clinical management of severe acute respiratory infection (SARI) when COVID-19 is suspected” interim guidance from the WHO [13 Mar. 2020] and local institutional guidelines) (World Health Organization, 2020).

Drug therapy of respiratory compromised patients may include, but not be limited to: antivirals, hydroxychloroquine, antibiotics, and supporting agents.

Prohibited Concomitant Medications. The following medications cannot be administered for the specified times prior to the initiation of study and for the duration of the study:
Use of any medications that are substrates of the transporters P-gp, BCRP, OATP1B1, OATP1B3, OCTs and with a narrow therapeutic index. Examples include digoxin, cyclosporine, leflunomide, mycophenolic acid, procainamide, sirolimus, everolimus, and dabigatran etexilate.
Note: Drugs considered to be substrates of these transporters and without a narrow therapeutic index (e.g., OATP1B1/3 substrate—statins, and OCTs substrate—metformin should be closely monitored for potential drug interactions while subjects are participating in the study. Additional examples of substrates of these transporters include methotrexate, sulfasalazine, leflunomide, rosuvastatin, aliskiren, ambrisentan, colchicine, cyclosporine, dabigatran etexilate, digoxin, everolimus, fexofenadine, methotrexate, ranolazine, rivaroxaban, saxagliptin, sirolimus, sitagliptin, talinolol, ticagrelor, tolvaptan, ambrisentan, atorvastatin, ezetimibe, fluvastatin, glyburide, rosuvastatin, simvastatin acid, pitavastatin, pravastatin, repaglinide, telmisartan, valsartan, olmesartan, mycophenolic acid, metformin, gabapentin, pramipexole, tramadol, varenicline.

The study compound dose in this study is 150 mg/day. If a subject experiences a Grade 3 drug related toxicity (except Grade≥3 AST, ALT, total bilirubin, or renal insufficiency; Grade≥2 peripheral neuropathies), then study compound dose may be reduced at the discretion of the treating physician.

All subjects will be monitored for adverse events during the study. All subjects discontinued from protocol-prescribed therapy for any reason will be followed for a period of 30 days following the last dose of study drug to collect safety data.

Pre-dose samples of peripheral blood, serum, buccal swabs, and nasopharyngeal swabs for pharmacodynamic assessments and exploratory analyses, as described in Section 6.1, will be collected in subjects of all study phases and treatment arms who consent to their collection. Peripheral blood and serum will be collected at screening and/or Day 1 of study treatment, whenever blood samples are collected for assessing disease status throughout treatment, at the end of treatment, and at follow-up as applicable (Table 2). Nasopharyngeal and buccal swabs will only be collected at screening and/or Day 1 of treatment, Day 5, Day 15, and Day 21.

Samples may be collected and processed as multiple samples to allow for the described and future retrospective analyses. One PK blood sample will be collected in subjects assigned to the study compound treatment at each of the visits on Days 3, 5, 8, 12, and 15; independent from the study compound dosing.

The date/time when subjects took their previous dose of the study compound, date/time of the study compound dosing on the day of clinical visit, and the date/time that blood is collected must be recorded in the eCRF. Explanation should be provided in the source documents and eCRF for any missed or mishandled samples.

Approximately 3 mL of whole blood will be drawn through an indwelling venous cannula or by venipuncture. Detailed instructions for sample collection, processing, storage, shipping and handling will be contained in a separate laboratory manual provided to the sites.

Overview of Key Efficacy Assessments: Safety of the study compound is evaluated based on the incidence of treatment-emergent adverse events (TEAEs) and changes in clinical laboratory parameters and vital signs. Safety assessments will be comprised of:

Record of adverse events (AEs) and serious adverse events (SAEs) at each study visit

Physical examination

Vital signs

Oxygen supplementation

Chest x-ray or CT

Laboratory assessments: hematology, serum chemistry, thiamine level, coagulation, urinalysis, serum/urine pregnancy tests

Electrocardiogram (ECG)

NEWS2 Scoring System. The NEWS2 is an aggregate scoring system in which a score is allocated to physiological measurements already recorded in routine practice when patients present to or are being monitored in hospital. Six simple physiological parameters form the basis of the scoring system:

respiration rate,

oxygen saturation,

systolic blood pressure,

pulse rate,

level of consciousness or new confusion,

temperature.

The NEWS2 will be assessed along with the daily assessments at approximately the same time each day, with each parameter being scored and the cumulative result indicating the clinical risk and urgency of response required, which will be entered into the appropriate eCRF.

Standard of care therapy for COVID-19 is currently evolving and will follow emerging guidance documents (e.g., the “Clinical management of severe acute respiratory infection (SARI) when COVID-19 is suspected” interim guidance from the WHO [13 Mar. 2020] and local institutional guidelines) (World Heath Organization, 2020). Drug therapy of respiratory compromised patients may include, but is not limited to, antivirals, hydroxychloroquine, antibiotics, or supporting agents.

All publications mentioned herein are hereby incorporated by reference in their entirety.

1. A method of treating a respiratory disease or disorder in a patient, comprising administering to the patient an effective amount of an MK2 inhibitor.

2. The method of claim 1, wherein the MK2 inhibitor is a compound of formula I: 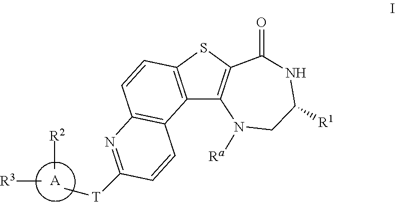
or a pharmaceutically acceptable salt thereof, wherein:
Ring A is phenyl, a 5-6 membered monocyclic heteroaryl ring having 1-3 nitrogen atoms, or an 8-14 membered bicyclic heteroaryl ring having 1-4 heteroatoms independently selected from nitrogen, oxygen, and sulfur;
T is a bivalent moiety selected from —N(R)—, —O—, —S—, —S(O)—, —SO₂—, —C(S)—, —Si(R⁴)₂—, —P(R⁵)—, —P(O)₂—, or a bivalent saturated straight or branched 1-3 membered hydrocarbon chain, wherein the hydrocarbon chain is optionally substituted with oxo or —OR;
each R is independently hydrogen or an optionally substituted C₁₋₆ aliphatic, or:
two R groups on the same nitrogen are taken together with the nitrogen to form a 3-7 membered saturated or partially unsaturated heterocyclic ring having 1-3 heteroatoms selected from nitrogen, oxygen, or sulfur;
R^{a} is hydrogen or an optionally substituted C₁₋₆ aliphatic;
R¹ is —R or —(CH₂)ₚR^{x};
p is 0, 1, 2, or 3;
R^{x} is —CN, —NO₂, halogen, —OR, —SR, —N(R)₂, —C(O)N(R)₂, —C(O)OR, —C(O)R, —N(R)C(O)R, —SO₂N(R)₂, or —N(R)SO₂;
R² is halogen, —CN, —SR^{y}, —S(O)R^{y}, —SO₂R^{y}, —OSO₂R^{y}, —OC(O)R^{y}, or —OP(O)₂OR^{y};
each R^{y} is independently selected from optionally substituted C₁₋₆ aliphatic or optionally substituted phenyl;
R³ is hydrogen, optionally substituted C₁₋₆ aliphatic, —CN, —NO₂, halogen, —OR, —N(R)₂, —C(O)N(R)₂, —C(O)OR, -Cy, —C(O)N(R)-Cy, —C(O)-Cy, —O-Cy, —O—(CH₂)ₙ-Cy, —(CH₂)ₙ—O-Cy, —(CH₂)ₘN(R)₂, —(CH₂)ₘOR, —N(R)-Cy, —N(R)—(CH₂)ₙ-Cy, —(CH₂)ₙ—N(R)-Cy, or —(CH₂)ₘ-Cy;
each R⁴ is independently hydrogen, —OR, C₁₋₆ aliphatic, phenyl, or a 5-6 membered heteroaryl ring having 1-3 heteroatoms independently selected from nitrogen, oxygen, and sulfur;
each R⁵ is independently —OR, C₁₋₆ aliphatic, phenyl, or a 5-6 membered heteroaryl ring having 1-3 heteroatoms independently selected from nitrogen, oxygen, and sulfur;
each of m and n is independently 0-4; and
each Cy is independently an optionally substituted ring selected from a 3-9 membered saturated or partially unsaturated monocyclic carbocyclic ring, a 3-9 membered saturated or partially unsaturated monocyclic heterocyclic ring having 1-3 heteroatoms independently selected from nitrogen, oxygen, and sulfur, phenyl, a 5-6 membered heteroaryl ring having 1-3 heteroatoms independently selected from nitrogen, oxygen, and sulfur, a 7-12 membered saturated or partially unsaturated fused or bridged bicyclic carbocyclic ring, or a 6-12 membered saturated or partially unsaturated fused or bridged bicyclic heterocyclic ring having 1-3 heteroatoms independently selected from nitrogen, oxygen, and sulfur.

3. The method of claim 1 or 2, wherein the MK2 inhibitor is: 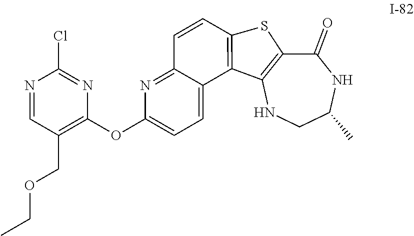 [SMILES: CCOCc1cnc(Cl)nc1Oc1ccc2c(ccc3sc4c(c32)NC[C@@H](C)NC4=O)n1]
or a pharmaceutically acceptable salt thereof.

4. The method of claim 1 or 2, wherein the MK2 inhibitor is 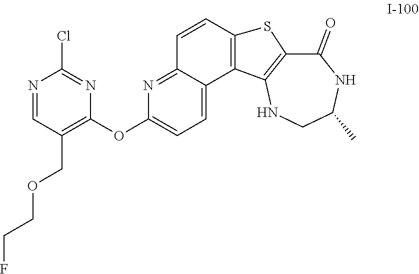 [SMILES: C[C@@H]1CNc2c(sc3ccc4nc(Oc5nc(Cl)ncc5COCCF)ccc4c23)C(=O)N1]
or a pharmaceutically acceptable salt thereof.

5. The method of claim any one of claims 1-4, wherein the MK2 inhibitor is administered once a day (“QD”).

6. The method of any of claims 2-5, wherein the MK2 inhibitor is administered in an amount of about 3 mg to about 1000 mg.

7. The method of any of claims 2-6, wherein the MK2 inhibitor is administered in an amount of about 3 mg to about 15 mg, about 10 mg to about 25 mg, about 15 mg to about 50 mg, about 25 mg to about 75 mg, about 50 mg to about 100 mg, about 75 mg to about 125 mg, about 100 mg to about 150 mg, or about 125 mg to about 200 mg.

8. The method of any of claims 2-7, wherein the MK2 inhibitor is administered in a unit dose comprising about 3 mg, about 5 mg, about 10 mg, about 15 mg, about 25 mg, about 50 mg, about 75 mg, or about 100 mg of the compound.

9. The method of any of claim 2-8, wherein the MK2 inhibitor is administered in an oral dosage form.

10. The method of claim 9, wherein the oral dosage form is a capsule.

11. The method of claim 9 or claim 10, wherein the MK2 inhibitor is administered in a spray-dried dispersion formulation.

12. The method of claim 11, wherein the spray-dried dispersion formulation comprises the compound, HPMCAS, microcrystalline cellulose, croscarmellose sodium, silicon dioxide, and magnesium stearate.

13. The method of any one of claims 1-12, wherein the respiratory disease or disorder is mediated by a coronavirus.

14. The method of any one of claims 1-12, wherein the respiratory disease or disorder is mediated by SARS-CoV-2.

15. The method of any one of claims 1-14, wherein the respiratory disease or disorder is COVID-19.

16. The method of any one of claims 1-15, wherein the patient has previously tested positive for SARS-CoV-2.

17. The method of any one of claims 1-16, wherein the patient has previously been hospitalized due to complications associated with COVID-19.

18. The method of any one of claims 1-17, wherein the patient is characterized by having one or more of the following: SpO2≤93% on room air, PaO2/FiO2≤300 mmHg without mechanical ventilation, respiratory rate≥30 per minute, or positive chest CT or X-ray for pneumonia.

19. The method of any one of claims 1-18, wherein the patient has one or more cardiovascular risk factors, or cardiovascular or thrombotic complications.

20. A method of modulating one or more pro-inflammatory cytokines in a patient suffering from COVID-19, the method comprising administering to the patient an effective amount of an MK2 inhibitor.

21. The method of claim 20, wherein the pro-inflammatory cytokines are selected from one or more of TNFα, IP10, IL-6, IL-18, IL-1RA, RANTES/CCL5, and CRP.

22. A method of treating a cytokine storm in a patent suffering from COVID-19, the method comprising administering to the patient an effective amount of an MK2 inhibitor.

23. The method of claim 22, wherein the cytokine storm comprises elevation of a cytokine selected from the group consisting of IL-1β, TNF-α, IL-6, IL-10, GM-CSF, and MCP-1, or combinations thereof

24. A method of reducing coronavirus replication in a patient, the method comprising administering to the patient an effective amount an MK2 inhibitor.

25. A method of inhibiting p38 pathway activation in a patient suffering from COVID-19, the method comprising administering to the patient an effective amount an MK2 inhibitor.

26. A method of treating pulmonary fibrosis in a patient suffering from COVID-19, the method comprising administering to the patient an effective amount of an MK2 inhibitor.

27. The method of any one of claims 20-26, wherein the MK2 inhibitor is a compound of formula I: 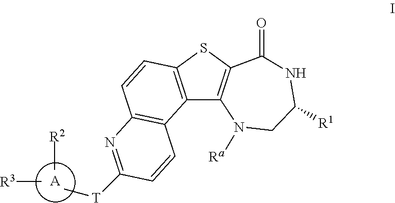
or a pharmaceutically acceptable salt thereof, wherein:
Ring A is phenyl, a 5-6 membered monocyclic heteroaryl ring having 1-3 nitrogen atoms, or an 8-14 membered bicyclic heteroaryl ring having 1-4 heteroatoms independently selected from nitrogen, oxygen, and sulfur;
T is a bivalent moiety selected from —N(R)—, —O—, —S—, —S(O)—, —SO₂—, —C(S)—, —Si(R⁴)₂—, —P(R⁵)—, —P(O)₂—, or a bivalent saturated straight or branched 1-3 membered hydrocarbon chain, wherein the hydrocarbon chain is optionally substituted with oxo or —OR;
each R is independently hydrogen or an optionally substituted C₁₋₆ aliphatic, or:
two R groups on the same nitrogen are taken together with the nitrogen to form a 3-7 membered saturated or partially unsaturated heterocyclic ring having 1-3 heteroatoms selected from nitrogen, oxygen, or sulfur;
R^{a} is hydrogen or an optionally substituted C₁₋₆ aliphatic;
R¹ is —R or —(CH₂)ₚR^{x};
p is 0, 1, 2, or 3;
R^{x} is —CN, —NO₂, halogen, —OR, —SR, —N(R)₂, —C(O)N(R)₂, —C(O)OR, —C(O)R, —N(R)C(O)R, —SO₂N(R)₂, or —N(R)SO₂;
R² is halogen, —CN, —SR^{y}, —S(O)R^{y}, —SO₂R^{y}, —OSO₂R^{y}, —OC(O)R^{y}, or —OP(O)₂OR^{y};
each R^{y} is independently selected from optionally substituted C₁₋₆ aliphatic or optionally substituted phenyl;
R³ is hydrogen, optionally substituted C₁₋₆ aliphatic, —CN, —NO₂, halogen, —OR, —N(R)₂, —C(O)N(R)₂, —C(O)OR, -Cy, —C(O)N(R)-Cy, —C(O)-Cy, —O-Cy, —O—(CH₂)ₙ-Cy, —(CH₂)ₙ—O-Cy, —(CH₂)ₘN(R)₂, —(CH₂)ₘOR, —N(R)-Cy, —N(R)—(CH₂)ₙ-Cy, —(CH₂)ₙ—N(R)-Cy, or —(CH₂)ₘ-Cy;
each R⁴ is independently hydrogen, —OR, C₁₋₆ aliphatic, phenyl, or a 5-6 membered heteroaryl ring having 1-3 heteroatoms independently selected from nitrogen, oxygen, and sulfur;
each R⁵ is independently —OR, C₁₋₆ aliphatic, phenyl, or a 5-6 membered heteroaryl ring having 1-3 heteroatoms independently selected from nitrogen, oxygen, and sulfur;
each of m and n is independently 0-4; and
each Cy is independently an optionally substituted ring selected from a 3-9 membered saturated or partially unsaturated monocyclic carbocyclic ring, a 3-9 membered saturated or partially unsaturated monocyclic heterocyclic ring having 1-3 heteroatoms independently selected from nitrogen, oxygen, and sulfur, phenyl, a 5-6 membered heteroaryl ring having 1-3 heteroatoms independently selected from nitrogen, oxygen, and sulfur, a 7-12 membered saturated or partially unsaturated fused or bridged bicyclic carbocyclic ring, or a 6-12 membered saturated or partially unsaturated fused or bridged bicyclic heterocyclic ring having 1-3 heteroatoms independently selected from nitrogen, oxygen, and sulfur.

28. The method claim 27, wherein the MK2 inhibitor is: 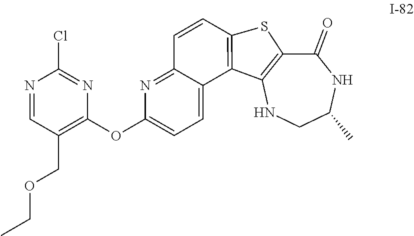 [SMILES: CCOCc1cnc(Cl)nc1Oc1ccc2c(ccc3sc4c(c32)NC[C@@H](C)NC4=O)n1]
or a pharmaceutically acceptable salt thereof.

29. The method claim 27, wherein the MK2 inhibitor is 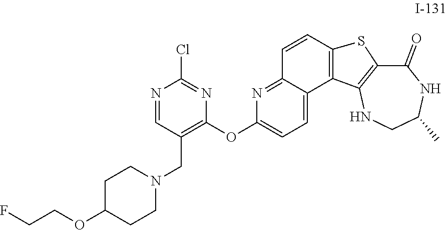 [SMILES: C[C@@H]1CNc2c(sc3ccc4nc(Oc5nc(Cl)ncc5CN5CCC(OCCF)CC5)ccc4c23)C(=O)N1]
or a pharmaceutically acceptable salt thereof.


SEQUENCE LISTING (2 sequences)

SEQ ID NO: 1 - protein, 400 aa, Homo sapiens
MLSNSQGQSPPVPFPAPAPPPQPPTPALPHPPAQPPPPPPQQFPQFHVKSGLQIKKNAIIDDYKVTSQVLGLGINGKVLQIFNKRTQEKFALKMLQDCPKARREVELHWRASQCPHIVRIVDVYENLYAGRKCLLIVMECLDGGELFSRIQDRGDQAFTEREASEIMKSIGEAIQYLHSINIAHRDVKPENLLYTSKRPNAILKLTDFGFAKETTSHNSLTTPCYTPYYVAPEVLGPEKYDKSCDMWSLGVIMYILLCGYPPFYSNHGLAISPGMKTRIRMGQYEFPNPEWSEVSEEVKMLIRNLLKTEPTQRMTITEFMNHPWIMQSTKVPQTPLHTSRVLKEDKERWEDVKEEMTSALATMRVDYEQIKIKKIEDASNPLLLKRRKKARALEAAALAH

SEQ ID NO: 2 - protein, 28 aa, Homo sapiens
NLYAGRKCLLIVMECLDGGELFSRIQDR